SEQUENCE LISTING (64 sequences)

SEQ ID NO: 1 - protein, 20 aa, synthetic construct
GGGGSGGGGSGGGGSASGGG

SEQ ID NO: 2 - protein, 216 aa, synthetic construct
MTYKLILNGKTLKGETTTEAVDAATAEKVFKQYANDNGVDGEWTYDDATKTFTVTEKPEVIDASELTPAVTTYKLVINGKTLKGETTTEAVDAATAEKVFKQYANDNGVDGEWTYDDATKTFTVTEKPEVIDASELTPAVTTYKLVINGKTLKGETTTKAVDAETAEKAFKQYANDNGVDGVWTYDDATKTFTVTEGGGGSGGGGSGGGGSASGGG

SEQ ID NO: 3 - protein, 42 aa, synthetic construct
MHGKTQATSGTIQSMHGKTQATSGTIQSMHGKTQATSGTIQS

SEQ ID NO: 4 - protein, 98 aa, synthetic construct
MHGKTQATSGTIQSMHGKTQATSGTIQSMHGKTQATSGTIQSMHGKTQATSGTIQSMHGKTQATSGTIQSMHGKTQATSGTIQSMHGKTQATSGTIQS

SEQ ID NO: 5 - protein, 12 aa, synthetic construct
WAGAKRLVLRRE

SEQ ID NO: 6 - protein, 10 aa, synthetic construct
HFSSWETQQG

SEQ ID NO: 7 - protein, 10 aa, synthetic construct
WYEKWQKANW

SEQ ID NO: 8 - protein, 8 aa, synthetic construct
VSGSSPDS

SEQ ID NO: 9 - protein, 36 aa, synthetic construct
SSKKSGSYSGSKGSRRILGGGGMHGKTQATSGTIQS

SEQ ID NO: 10 - protein, 30 aa, synthetic construct
MSPHPHPRHHHTGGGGMHGKTQATSGTIQS

SEQ ID NO: 11 - protein, 30 aa, synthetic construct
RGRRRRLSCRLLGGGGMHGKTQATSGTIQS

SEQ ID NO: 12 - protein, 30 aa, synthetic construct
DSARGFKKPGKRGGGGMHGKTQATSGTIQS

SEQ ID NO: 13 - protein, 30 aa, synthetic construct
HPPMNASHPHMHGGGGMHGKTQATSGTIQS

SEQ ID NO: 14 - protein, 30 aa, synthetic construct
HKDHHANQHVHMGGGGMHGKTQATSGTIQS

SEQ ID NO: 15 - protein, 108 aa, synthetic construct
PTTGSCAVTYTANGWSGGFTAAVTLTNTGTTALSGWTLGFAFPSGQTLTQGWSARWAQSGSSVTATNEAWNAVLAPGASVEIGFSGTHTGTNTAPATFTVGGATCTTR

SEQ ID NO: 16 - protein, 108 aa, synthetic construct
SGPAGCQVLWGVNQWNTGFTANVTVKNTSSAPVDGWTLTFSFPSGQQVTQAWSSTVTQSGSAVTVRNAPWNGSIPAGGTAQFGFNGSHTGTNAAPTAFSLNGTPCTVG

SEQ ID NO: 17 - protein, 12 aa, synthetic construct
RAFIASRRIRRP

SEQ ID NO: 18 - protein, 9 aa, synthetic construct
RIIIRRIRR

SEQ ID NO: 19 - protein, 196 aa, unidentified
  source 1..196: note = Streptococcus sp.
MTYKLILNGKTLKGETTTEAVDAATAEKVFKQYANDNGVDGEWTYDDATKTFTVTEKPEVIDASELTPAVTTYKLVINGKTLKGETTTEAVDAATAEKVFKQYANDNGVDGEWTYDDATKTFTVTEKPEVIDASELTPAVTTYKLVINGKTLKGETTTKAVDAETAEKAFKQYANDNGVDGVWTYDDATKTFTVTE

SEQ ID NO: 20 - protein, 314 aa, synthetic construct
MTYKLILNGKTLKGETTTEAVDAATAEKVFKQYANDNGVDGEWTYDDATKTFTVTEKPEVIDASELTPAVTTYKLVINGKTLKGETTTEAVDAATAEKVFKQYANDNGVDGEWTYDDATKTFTVTEKPEVIDASELTPAVTTYKLVINGKTLKGETTTKAVDAETAEKAFKQYANDNGVDGVWTYDDATKTFTVTEGGGGSGGGGSGGGGSASGGGMHGKTQATSGTIQSMHGKTQATSGTIQSMHGKTQATSGTIQSMHGKTQATSGTIQSMHGKTQATSGTIQSMHGKTQATSGTIQSMHGKTQATSGTIQS

SEQ ID NO: 21 - protein, 180 aa, synthetic construct
MDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQGGGGSGGGGSGGGGSASGGG

SEQ ID NO: 22 - protein, 160 aa, synthetic construct
MDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 23 - protein, 278 aa, synthetic construct
MDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQGGGGSGGGGSGGGGSASGGGMHGKTQATSGTIQSMHGKTQATSGTIQSMHGKTQATSGTIQSMHGKTQATSGTIQSMHGKTQATSGTIQSMHGKTQATSGTIQSMHGKTQATSGTIQS

SEQ ID NO: 24 - protein, 188 aa, synthetic construct
MDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQGGGGSGGGGSGGGGSASGGGVSGSSPDS

SEQ ID NO: 25 - protein, 12 aa, synthetic construct
RGRRRRLSCRLL

SEQ ID NO: 26 - protein, 192 aa, synthetic construct
MDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQGGGGSGGGGSGGGGSASGGGRGRRRRLSCRLL

SEQ ID NO: 27 - protein, 288 aa, synthetic construct
MDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQGGGGSGGGGSGGGGSASGGGPTTGSCAVTYTANGWSGGFTAAVTLTNTGTTALSGWTLGFAFPSGQTLTQGWSARWAQSGSSVTATNEAWNAVLAPGASVEIGFSGTHTGTNTAPATFTVGGATCTTR

SEQ ID NO: 28 - protein, 288 aa, synthetic construct
MDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQGGGGSGGGGSGGGGSASGGGSGPAGCQVLWGVNQWNTGFTANVTVKNTSSAPVDGWTLTFSFPSGQQVTQAWSSTVTQSGSAVTVRNAPWNGSIPAGGTAQFGFNGSHTGTNAAPTAFSLNGTPCTVG

SEQ ID NO: 29 - protein, 518 aa, synthetic construct
MHHHHHHENYLFQGQVQLVESGAEVKKPGESLKISCKGSGYSFPSYWINWVRQMPGKGLEWMGMIYPADSDTRYSPSFQGHVTISADKSINTAYLQWAGLKASDTAIYYCARLGIGGRYMSRWGQGTLVTVSSAPTPTPTTPTPTPTTPTPTPSTEVQLVESGGDLVKPGGSLKLSCAASGFTFSSFAMSWVRQTPERKLEWVATVGTGGFYTFYPDNVEGRFTVSRDNAKNTLYLQMSSLRSEDTAIYYCVRREEAFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCTSKPGGGGSGGGGSGGGGSASGGGDIVLTQAAFSNPVTLGTSASISCRSTKSLLHSNGITFLYWYLQRPGQSPQLLISQMSTLASGVPDRFSSSGSGTDFTLRISRVEAEDVGVYYCAQNLELPYTFGGGTKLEIKRADAAPTVS

SEQ ID NO: 30 - protein, 192 aa, synthetic construct
MDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQGGGGSGGGGSGGGGSASGGGRAFIASRRIRRP

SEQ ID NO: 31 - protein, 189 aa, synthetic construct
MDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQGGGGSGGGGSGGGGSASGGGRIIIRRIRR

SEQ ID NO: 32 - protein, 222 aa, synthetic construct
EVQLVESGGDLVKPGGSLKLSCAASGFTFSSFAMSWVRQTPERKLEWVATVGTGGFYTFYPDNVEGRFTVSRDNAKNTLYLQMSSLRSEDTAIYYCVRREEAFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCTSKP

SEQ ID NO: 33 - protein, 121 aa, synthetic construct
DIVLTQAAFSNPVTLGTSASISCRSTKSLLHSNGITFLYWYLQRPGQSPQLLISQMSTLASGVPDRFSSSGSGTDFTLRISRVEAEDVGVYYCAQNLELPYTFGGGTKLEIKRADAAPTVS

SEQ ID NO: 34 - protein, 291 aa, synthetic construct
MHHHHHHENLYFQGDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQGGGGSGGGGSGGGGSASGGGMHGKTQATSGTIQSMHGKTQATSGTIQSMHGKTQATSGTIQSMHGKTQATSGTIQSMHGKTQATSGTIQSMHGKTQATSGTIQSMHGKTQATSGTIQS

SEQ ID NO: 35 - protein, 16 aa, synthetic construct
HPPMNASHPHMHGGGG

SEQ ID NO: 36 - protein, 18 aa, synthetic construct
SSKKSGSYSGSKGSRRIL

SEQ ID NO: 37 - protein, 12 aa, synthetic construct
MSPHPHPRHHHT

SEQ ID NO: 38 - protein, 12 aa, synthetic construct
DSARGFKKPGKR

SEQ ID NO: 39 - protein, 12 aa, synthetic construct
HPPMNASHPHMH

SEQ ID NO: 40 - protein, 12 aa, synthetic construct
HKDHHANQHVHM

SEQ ID NO: 41 - protein, 12 aa, synthetic construct
NPSSLFRYLPSD

SEQ ID NO: 42 - protein, 12 aa, synthetic construct
HSSYWYAFNNKT

SEQ ID NO: 43 - protein, 12 aa, synthetic construct
EAHVMHKVAPRP

SEQ ID NO: 44 - protein, 12 aa, synthetic construct
HHGHSPTSPQVR

SEQ ID NO: 45 - protein, 30 aa, synthetic construct
MVMPGDNIKMVVTLIHPIAMDDGLRFAIRE

SEQ ID NO: 46 - protein, 45 aa, synthetic construct
VGPNNVPYIVATITSNSAGGQPVSLANLKAMYSIAKKYDIPVVMD

SEQ ID NO: 47 - protein, 31 aa, synthetic construct
NNSWTRVAFAGLKFQDVGSFDYGRNYGVVYD

SEQ ID NO: 48 - protein, 120 aa, synthetic construct
QVQLVESGAEVKKPGESLKISCKGSGYSFPSYWINWVRQMPGKGLEWMGMIYPADSDTRYSPSFQGHVTISADKSINTAYLQWAGLKASDTAIYYCARLGIGGRYMSRWGQGTLVTVSSA

SEQ ID NO: 49 - protein, 228 aa, synthetic construct
MTYKLILNGKTLKGETTTEAVDAATAEKVFKQYANDNGVDGEWTYDDATKTFTVTEKPEVIDASELTPAVTTYKLVINGKTLKGETTTEAVDAATAEKVFKQYANDNGVDGEWTYDDATKTFTVTEKPEVIDASELTPAVTTYKLVINGKTLKGETTTKAVDAETAEKAFKQYANDNGVDGVWTYDDATKTFTVTEGGGGSGGGGSGGGGSASGGGHPPMNASHPHMH

SEQ ID NO: 50 - protein, 16 aa, synthetic construct
HPPMNASHPHMHGGGG

SEQ ID NO: 51 - protein, 338 aa, synthetic construct
MDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQGGGGSGGGGSGGGGSASGGGSSKKSGSYYSYGTKKSGSYSGYSTKKSASRRILSSKKSGSYSGYSTKKSGSRRILSSKKSGSYSGSKGSKRRILSSKKSGSYSGSKGSKRRNLSSKKSGSYSGSKGSKRRILSSKKSGSYSGSKGSKRRNLSSKKSGSYSGSKGSKRRILSGGLRGSM

SEQ ID NO: 52 - protein, 192 aa, synthetic construct
MDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQGGGGSGGGGSGGGGSASGGGNPSSLFRYLPSD

SEQ ID NO: 53 - protein, 192 aa, synthetic construct
GDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQGGGGSGGGGSGGGGSASGGGHSSYWYAFNNKT

SEQ ID NO: 54 - protein, 205 aa, synthetic construct
MHHHHHHENLYFQGDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQGGGGSGGGGSGGGGSASGGGEAHVMHKVAPRP

SEQ ID NO: 55 - protein, 205 aa, synthetic construct
MHHHHHHENLYFQGDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQGGGGSGGGGSGGGGSASGGGHHGHSPTSPQVR

SEQ ID NO: 56 - protein, 223 aa, synthetic construct
MHHHHHHENLYFQGDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQGGGGSGGGGSGGGGSASGGGMVMPGDNIKMVVTLIHPIAMDDGLRFAIRE

SEQ ID NO: 57 - protein, 238 aa, synthetic construct
MHHHHHHENLYFQGDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQGGGGSGGGGSGGGGSASGGGVGPNNVPYIVATITSNSAGGQPVSLANLKAMYSIAKKYDIPVVMD

SEQ ID NO: 58 - protein, 224 aa, synthetic construct
MHHHHHHENLYFQGDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQGGGGSGGGGSGGGGSASGGGNNSWTRVAFAGLKFQDVGSFDYGRNYGVVYD

SEQ ID NO: 59 - protein, 314 aa, synthetic construct
MHHHHHHENYLFQGQVQLVESGAEVKKPGESLKISCKGSGYSFPSYWINWVRQMPGKGLEWMGMIYPADSDTRYSPSFQGHVTISADKSINTAYLQWAGLKASDTAIYYCARLGIGGRYMSRWGQGTLVTVSSAPTPTPTTPTPTPTTPTPTPSTDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 60 - protein, 159 aa, synthetic construct
DPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 61 - protein, 20 aa, synthetic construct
TPTPTTPTPTPTTPTPTPST

SEQ ID NO: 62 - protein, 302 aa, synthetic construct
MHHHHHHENLYFQGDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQPTPTPTTPTPTPTTPTPTPSTSGPAGCQVLWGVNQWNTGFTANVTVKNTSSAPVDGWTLTFSFPSGQQVTQAWSSTVTQSGSAVTVRNAPWNGSIPAGGTAQFGFNGSHTGTNAAPTAFSLNGTPCTVG

SEQ ID NO: 63 - protein, 301 aa, synthetic construct
MHHHHHHENLYFQGSGPAGCQVLWGVNQWNTGFTANVTVKNTSSAPVDGWTLTFSFPSGQQVTQAWSSTVTQSGSAVTVRNAPWNGSIPAGGTAQFGFNGSHTGTNAAPTAFSLNGTPCTVGGGGGSGGGGSGGGGSASGGGDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 64 - protein, 302 aa, synthetic construct
MHHHHHHENLYFQGSGPAGCQVLWGVNQWNTGFTANVTVKNTSSAPVDGWTLTFSFPSGQQVTQAWSSTVTQSGSAVTVRNAPWNGSIPAGGTAQFGFNGSHTGTNAAPTAFSLNGTPCTVGPTPTPTTPTPTPTTPTPTPSTDPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

(19) United States
(12) Patent Application Publication
(10) Pub. No.: US 2024/0036044 A1
(43) Pub. Date: Feb. 1, 2024

(54) DUAL-AFFINITY PROBES AND SYSTEMS FOR ANALYTE DETECTION

(71) Applicant: Gemina Laboratories Ltd., Vancouver (CA)

(72) Inventors: Robert Crandall Greene, North Vancouver (CA); Christine BUERKI, Burnaby (CA)

(21) Appl. No.: 18/181,325

(22) Filed: Mar. 9, 2023

Related U.S. Application Data
(60) Provisional application No. 63/318,365, filed on Mar. 9, 2022.

(51) Int. Cl. G01N 33/569

(52) U.S. Cl. CPC G01N 33/56983

(57) ABSTRACT

The present document describes a dual-affinity probe comprising an organic or inorganic surface binding peptide and a target-specific capture element, which may bind to various targets, such as pathogens. This document further describes uses of the dual-affinity probe, and kits to determine the presence of and/or quantity of a target in a sample. In particular embodiments, the dual-affinity probe is specific for SARS-CoV-2 (Spike or Nucleocapsid) protein and may be used to determine whether a subject is infected with SARS-CoV-2.

### CROSS-REFERENCE TO RELATED APPLICATIONS

This application claims priority to U.S. Provisional Application No. 63/318,365, filed Mar. 9, 2022, the disclosure of which is herein incorporated by reference in its entirety for all purposes.

### FIELD OF THE INVENTION

The subject matter disclosed generally relates to genetic assemblies of inorganic and organic binding entities to functionalize various biosensors for the detection of any pathogens of interest.

### REFERENCE TO AN ELECTRONIC SEQUENCE LISTING

The contents of the electronic sequence listing (GEML_002_01US_SeqList_ST26.xml; Size: 64,373 bytes; and Date of Creation: Mar. 7, 2023) are herein incorporated by reference in its entirety.

### BACKGROUND OF THE INVENTION

Pathogen detection for many applications primarily relies on three different technologies: i) culture-based methods, ii) immunoassays (such as enzyme linked immunosorbent assay (ELISA)) and iii) polymerase chain reaction (PCR)-based methods. While cultures and ELISA are sensitive methods for pathogen detection, their main drawback is turnaround time with cultures taking days to generate a result. Although PCR is very sensitive, and faster than the culture-based methods and immunoassays, it requires technical expertise and a multi-step process to first isolate DNA or RNA for analysis. Furthermore, PCR is not able to differentiate between viable and nonviable pathogens.

Human coronaviruses are positive sense, single stranded RNA viruses. There are seven types of coronaviruses known to infect humans. Patients infected with these viruses develop respiratory symptoms of various severity. HCoV-229E and HCoV-OC43 are well known and cause common colds. Five other coronaviruses lead to more severe respiratory tract infection, which can potentially be lethal. Since 2000, there have been three major world-wide health crises caused by coronaviruses, the 2003 SARS outbreak, the 2012 MERS outbreak, and the most recent 2019 COVID-19 outbreak.

Biosensors, analytical devices that combine a biological component with a physiochemical detector for the detection of a chemical substance, can be categorized based on their capture elements (enzyme-based, immunosensors using antibodies, DNA biosensors, etc.), or their transducers (thermal, piezoelectric biosensors, etc.). The best-known biosensors are the lateral flow-based pregnancy test and the electrochemical glucose biosensors.

The immobilization of the capture elements or bioreceptors on the surface is of great importance as they not only functionalize but also determine the sensitivity of the biosensor. There are two groups of immobilization methods: irreversible and reversible. Irreversible immobilization includes covalent binding, cross-linking and entrapment, while reversible methods include random adsorption, bioaffinity (biotin/streptavidin and protein A/G), chelation/metal binding and disulfide bonds (LIEBANA; DRAGO, 2016).

Antibodies are sensing biomolecules often used for the clinical application of biosensors. The easiest way of preparing a sensor with antibodies is random adsorption. Random adsorption, however, is associated with the denaturation of proteins, very low stability and random orientation, thus affecting the performance of the biosensor. The most widely used method for antibody immobilization is through covalent binding which, however, also results in random orientations of the antibodies as the amino/carboxyl groups used in the covalent bonds are uniformly distributed on the antibody.

There is a need in the art for improved biosensors. The present disclosure addresses this need by providing dual-affinity probes and biosensors for the detection of analytes, including but not limited to pathogens, with the sensitivity and specificity needed in various applications, including in a point of care setting.

### SUMMARY OF THE INVENTION

The disclosure provides dual affinity probes and related methods of use, e.g., to determine the presence of and/or amount or quantity of a target analyte in a sample. The dual affinity probes comprise: (i) an inorganic surface binding element, and (ii) a capture element.

According to an embodiment of the invention, there is provided a dual-affinity immunoprobe for detecting an analyte, e.g., a pathogen, in a sample, the immunoprobe including an inorganic surface binding peptide and an analyte-specific capture element. In embodiments, the analyte-specific capture element is an organic binding entity specific for the analyte, e.g., pathogen. In other embodiments, the capture element is selected from protein G from Streptococcus, streptavidin from Streptomyces, a single chain variable fragment, a Fab fragment, or an antibody. In particular embodiments, the capture element specifically binds to the analyte, e.g., pathogen.
In certain embodiments, the capture element is connected to the inorganic surface binding peptide via a linker sequence. In still other embodiments, the inorganic surface binding peptide binds specifically to a biosensor material selected from the group consisting of gold, silica, silver, cellulose (e.g., nitrocellulose), plastic, polystyrene, and graphene.

In embodiments, the analyte-specific capture element specifically binds the analyte. In embodiments, the analyte-specific capture element is a pathogen-specific capture element that specifically binds the pathogen. In some embodiments, the pathogen is SARS-CoV-2.

In embodiments of the invention, there is provided a dual-affinity probe wherein an inorganic surface binding peptide comprises gold-, silver-, silica-, plastic-, cellulose-, polystyrene-, or graphene-binding peptides fused to protein G or streptavidin, and a capture element comprises antibodies that specifically binds a target analyte.

In embodiments of the invention, there is provided a dual-affinity probe wherein an inorganic surface binding peptide comprises gold-, silver-, silica-, plastic-, cellulose-, polystyrene-, or graphene-binding peptides fused to protein G or streptavidin, and a capture element comprises S or N antigen targeting antibodies specific for SARS-CoV-2 Spike (S) antigen or SARS-CoV-2 Nucleocapsid (N) antigen.

In embodiments, the inorganic surface binding peptide is selected from Table 1 herein. In another embodiment, the inorganic surface binding peptide is selected from EMT014, EMT015, EMT016, EMT017, EMT018, EMT019, EMT020, EMT021, EMT022, EMT023, EMT024, EMT025. In another embodiment, the inorganic surface binding peptide is selected from cellulose binding motif 1, cellulose binding motif 2, polystyrene binding motif 1, polystyrene binding motif 2, and silica binding motif.

According to an embodiment, there is provided a platform using gold-binding peptides fused to protein G and coupled to S antigen targeting antibodies for detecting novel coronavirus SARS-CoV-2 via SARS-CoV-2 Spike (S) antigen in some embodiments, and Nucleocapsid (N) antigen in other embodiments.

According to another embodiment, there is provided a platform using silica-binding peptides fused to protein G and coupled to N antigen targeting antibodies for detecting novel coronavirus SARS-CoV-2 via SARS-CoV-2 Nucleocapsid (N) antigen.

According to yet another embodiment, there is provided a platform using gold-binding peptides fused to protein G and coupled to S and N antigen targeting antibodies for detecting novel coronavirus SARS-CoV-2 via SARS-CoV-2 Spike (S) antigen.

According to another embodiment, there is provided a platform using silica-binding peptides fused to protein G and coupled to S and N antigen targeting antibodies for detecting novel coronavirus SARS-CoV-2 via SARS-CoV-2 Nucleocapsid (N) antigen.

According to an embodiment, there is provided a platform using gold-binding peptides fused to streptavidin and coupled to S and N antigen targeting antibodies for detecting novel coronavirus SARS-CoV-2 via SARS-CoV-2 Spike (S) antigen in some embodiments, and Nucleocapsid (N) antigen in other embodiments.

According to an embodiment, there is provided a platform using cellulose-binding peptides, silica-binding peptides fused to streptavidin, or polystyrene-binding peptides which are then fused to streptavidin and coupled to S and N antigen targeting antibodies for detecting novel coronavirus SARS-CoV-2 via SARS-CoV-2 Spike (S) antigen in some embodiments, and Nucleocapsid (N) antigen in other embodiments.

In embodiments, the platform detects the analyte, e.g., pathogens, via quartz crystal microbalance with dissipation (QCM-D). In other embodiments, the platform detects the analyte, e.g., pathogens, via surface plasmon resonance (SPR). In still other embodiments, the platform detects the analyte, e.g., pathogens, via lateral flow.

In a specific embodiment, the invention may be a dual-affinity probe for detecting an analyte, e.g., a pathogen, in a sample, the probe comprising a surface binding moiety (SBM), wherein the surface binding moiety is optionally an inorganic surface binding peptide (ISBP), and a capture element (CE). In a specific embodiment, the capture element (CE) is connected to the inorganic surface binding peptide via one or more linker (LI), wherein each LI may independently be a single bond or an amino acid sequence. In certain embodiments, the one or more linkers are passive linkers and/or active linkers. In a specific embodiment the probe has the following formula (I) or formula (II):

SBM-LI-CE (Ia) or

CE-LI-SBM (IIa)

The capture element CE may be an organic binding entity specific for the analyte, wherein the analyte is optionally a pathogen or a fragment thereof. In a specific embodiment, the capture element comprises an antibody or an antigen-binding fragment thereof, optionally a single chain variable fragment (scFv) or a Fab fragment; or an antigen.

In another embodiment, the LI comprises one or more linkers, wherein each linker is independently a single bond, such as an ionic or covalent or non-covalent bond, or is selected from one or more of the group consisting of: a peptide or amino acid linker, an amino acid sequence comprising protein G from Streptococcus, and an amino acid sequence comprising streptavidin from Streptomyces. In another embodiment, LI comprises or is the protein G from Streptococcus or streptavidin from Streptomyces.

In another embodiment, the SBM or ISBP binds specifically to a biosensor material selected from the group consisting of gold, silica, silver, cellulose, nitrocellulose, plastic, polystyrene and graphene. In a further embodiment, the biosensor material is selected from the group consisting of gold, cellulose, silica and polystyrene.

In a more specific embodiment, the SBM or ISBP is selected from the group consisting of a binding peptide, a protein, an antibody with an affinity to the inorganic surface, or an immunogenic fragment thereof, optionally a single chain variable fragment (scFv) or a Fab fragment. In a specific embodiment, the SBM or ISBP is a binding peptide. In another embodiment, the ISBP is selected from the group consisting of any peptide sequence of Table 1 herein.

In another embodiment, the SBM or ISBP is an antibody, a single chain variable fragment from an antibody, or a Fab fragment. In a specific embodiment, the SBM or ISBP comprises a gold binding motif. In a further specific embodiment, the gold binding motif is a V_{H} gold binding motif. In another embodiment the SBM or ISBP is an antibody. In a more specific embodiment, the SBM or ISBP is an antibody specific to binding gold.

In a specific embodiment, the dual affinity probe for detecting an analyte in a sample may comprise i) a surface binding moiety (SBM) fused or bound to a non-analyte capture element (NACE), or ii) a surface particle fused or bound to a NACE or a capture element (CE).

In a specific embodiment, the surface particle is bound to a surface binding peptide (SBP), wherein the SBP is fused or bound to a non-analyte capture element (NACE), wherein the NACE is additionally fused or bound to a CE and the DAP has the following Formula I or Formula II:

(Surface particle)-SBP-NACE-CE (Formula Ic); or

(Surface particle)-SBP-CE (Formula IIc).

In another embodiment of the dual-affinity probes, the CE is an antibody, or an antigen-binding fragment thereof, optionally an scFv or a Fab. In a specific embodiment, the CE is an antibody or an antigen-binding fragment thereof, wherein the antibody or antigen-binding fragment thereof is conjugated with biotin, and the LI is an amino acid sequence comprising streptavidin from Streptomyces. In another specific embodiment, the CE is an antibody or an antigen-binding fragment thereof, and the LI is an amino acid sequence comprising protein G from Streptococcus. In a specific embodiment, the CE is an S or N antigen targeting antibody specific for SARS-CoV-2 Spike (S) antigen or SARS-CoV-2 Nucleocapsid (N) antigen, or an antigen-binding fragment thereof.

In another specific embodiment, the CE is an antigen. In another specific embodiment, the CE is an antigen fused to a linker or SBM/ISBP. In another specific embodiment, the CE antigen is biotinylated and binds to a streptavidin linker. In another specific embodiment, the CE is an antigen that binds to an antibody (or antibodies), wherein the antibody or antibodies are the intended analyte for detection. In a specific embodiment, the antigen protein is SARS-CoV-2 Spike and/or SARS-CoV-2 Nucleocapsid proteins. In another specific embodiment, the antigen binds to and detects antibodies. In another embodiment, the antibody or antibodies are a targeting antibody specific for SARS-CoV-2 Spike (S) antigen or SARS-CoV-2 Nucleocapsid (N) antigen, or an antigen-binding fragment thereof. In another embodiment of the dual-affinity probes, LI is a single bond, such as a covalent bond, or a peptide or amino acid linker. In a specific embodiment, the amino acid linker is a passive linker to allow, for example, space between the CE and ISBP, or to provide some rigidity or flexibility to the CE and SBM or ISBP combination. In a specific embodiment, the dual-affinity probe is a single fusion protein. In another embodiment, the CE and the SBM or ISBP is independently an antibody, or an antigen-binding fragment thereof, optionally a single chain variable fragment. In a specific embodiment, the ISBP is the single chain variable fragment. In a more specific embodiment, the single chain variable fragment is a V_{H} gold binding motif. In another embodiment, the CE is a single chain variable fragment from an antibody. In a more specific embodiment, the SBM or ISBP and the CE are fused as a bispecific antibody fragment. In a specific embodiment, the SBM or ISBP is a single chain variable fragment that is a V_{H} gold binding motif, and the CE is a single chain variable fragment specific to an antigen. In another embodiment, one or both of the CE and the SBM or ISBP is an antibody. In a specific embodiment, the CE and the ISBP are fused to form a bispecific immunoglobulin A. In a specific embodiment, the ISBP is specific for gold, silica, silver, cellulose, plastic, polystyrene, or graphene. In a further specific embodiment, the ISBP is specific for gold. In another embodiment, the CE is specific to an antigen of SARS-CoV-2. In a specific embodiment, the CE is specific for SARS-CoV-2 Spike (S) antigen or SARS-CoV-2 Nucleocapsid (N) antigen. In another embodiment, the CE is an S or N antigen targeting antibody specific for SARS-CoV-2 Spike (S) antigen or SARS-CoV-2 Nucleocapsid (N) antigen, or an antigen-binding fragment thereof.

The present invention also includes compositions comprising one or more dual-affinity probes. In particular embodiments, the compositions are liquid compositions, wherein the dual affinity probes are present, e.g., in a buffered solution. In other embodiments, the compositions are solid compositions to which one or more dual affinity probes are bound or immobilized on. In another embodiment, the compositions may be added to a surface of an assay, system, or kit.

The present invention may also include dual-affinity probes incorporated into a specific system or diagnostic system, such as for a specific point of care diagnostic system. Any diagnostic system comprising a dual-affinity probe may be used. For example, in a specific embodiment, the system includes analysis performed on a quartz crystal microbalance, a surface plasmon resonance (SPR), and/or performed via lateral flow. In a specific embodiment, the system is used for the detection of an analyte, e.g., a pathogen, of known sequence, comprising a dual-affinity probe. In a specific embodiment, the dual-affinity probe may be any probe described herein. The system may for example include any dual-affinity probe bound to an inorganic surface biosensor material selected from the group consisting of gold, silica, silver, cellulose, plastic, and graphene. In a specific system, the dual-affinity probe capture element is specific for SARS-CoV-2 (Spike or Nucleocapsid) protein. A specific embodiment may include a system or diagnostic kit for detecting an analyte in a sample, the kit comprising: a) a surface; b) a surface binding moiety (SBM) bound to at least a portion of the surface; c) a reagent for collecting the sample; and d) an analyte composition comprising a set of at least one dual affinity probe. In a specific embodiment, the kit or system may comprise: a) a surface; b) a surface binding moiety (SBM) bound to at least a portion of the surface, wherein the SBM comprises an surface binding peptide (SBP), and/or optionally a non-analyte capture element (NACE); c) a reagent for collecting the sample; and d) an analyte composition comprising a set of at least one dual affinity probe as described herein.

The present invention also comprises methods of analyte, e.g., pathogen, detection using dual-affinity probes to analyze a medium for an analyte, e.g., a pathogen. In a specific embodiment, the dual-affinity probes may be any dual-affinity probe described herein. In another embodiment of the methods, the analysis is performed on a quartz crystal microbalance with dissipation (QCM-D), using surface plasmon resonance (SPR), and/or performed via lateral flow.

In a specific embodiment of the methods of the present invention, the method includes determining the presence of and/or quantifying an analyte, e.g., a pathogen, in a test sample, comprising: a) contacting a test sample with a dual-affinity probe, wherein the dual-affinity probe comprises an inorganic surface binding polypeptide and an analyte-specific capture element, under conditions and for a time sufficient for analyte present in the test sample to bind to the analyte-specific capture element, thereby forming complexes comprising the analyte bound to the dual-affinity probe; and b) determining the presence or absence of and/or the quantity of the complexes or analyte present in the complexes; c) wherein the presence of the complexes or the analyte in the complexes indicates the presence of the analyte in the test sample, and wherein the quantity of the complexes or the analyte in the complexes indicates the quantity of analyte present in the test sample, thereby determining the presence of and/or quantifying the analyte in the test sample.

In a specific embodiment of the methods, the test sample is a biological sample obtained from a subject. In a specific embodiment, the subject is a mammal, optionally a human. In another embodiment, the biological sample comprises serum, plasma, whole blood, saliva, mucus, nasal fluid, cerebrospinal fluid, sweat, urine, or a combination thereof. In another embodiment, the analyte is a pathogen. In a specific embodiment, the pathogen is a virus, a bacterium, a fungus, a protozoan, a worm, or a prion. In a specific embodiment, the virus is a SARS-CoV-2 virus. In a further specific embodiment, the analyte-specific capture element comprises antibodies, or antigen-binding fragments thereof, specific for a SARS-CoV-2 Spike (S) antigen or a SARS-CoV-2 Nucleocapsid (N) antigen.

In another embodiment of the methods, the inorganic surface binding polypeptide or surface binding peptide comprises one or more gold-, silver-, silica-, plastic-, cellulose- or graphene-binding peptides. In another embodiment, the inorganic surface binding polypeptide comprises a peptide selected from any peptide sequence of Table 1 or Table 1a herein. In another embodiment, the dual-affinity probe is bound to a surface, such as an inorganic surface. In another embodiment, the surface is a biosensor material selected from the group consisting of gold, silica, silver, cellulose, plastic, and graphene. In a specific embodiment of the methods, the specific contacting and/or determining is performed using a quartz crystal microbalance, surface plasmon resonance (SPR) or via lateral flow. In a specific embodiment, the methods may include a method of determining the presence of and/or quantifying an analyte in a sample from a subject, by testing the sample with any one of the diagnostic kits or systems described herein, comprising: a) contacting a test sample with the reagent; b) applying the test sample with the reagent to the surface thereby allowing the test sample to flow laterally on the surface thereby contacting the DAP composition on the surface; c) allowing for the analyte present in the test sample to bind directly to the CE, thereby forming complexes comprising the analyte bound to the DAP; d) allowing for the analyte complexed to the DAP to further flow laterally on the surface, thereby complexing to the surface binding moiety (SBM); and e) wherein the presence of the DAP complexed to the SBM determines the presence and/or quantity of the analyte in the test sample.

### BRIEF DESCRIPTION OF THE DRAWINGS

Further features and advantages of the present disclosure will become apparent from the following detailed description, taken in combination with the appended drawings, in which:

FIGS. 1A-1C illustrate the expression and purity of the gold-binding and silica-binding ISBP on Coomassie-stained SDS-PAGE gels. 2 μg of BSA was added in lane 1 as a loading control. FIG. 1A shows an ISBP-free fusion protein, FIG. 1B shows a Gold-binding fusion protein, and FIG. 1C shows a Silica-binding fusion protein.

FIG. 2 illustrates the mass and thickness of the layers of captured pathogen formed during the SARS-CoV-2 Spike protein antigen capture with the SARS-CoV-2 Spike antibody using QCM-D on a gold sensor (left panel). The right panel illustrates the same experiment using a SARS-CoV-2 Nucleocapsid protein antigen and SARS-CoV-2 Spike antibody. The left y-axis indicates thickness (nm) of the layer and the right y-axis, mass in ng/cm² deposited. The x-axis is time in seconds.

FIG. 3 is an SPR sensorgram of the immobilization of gold-binding fusion protein onto a gold sensor surface. ISBP-free fusion protein and “buffer only” were run in parallel as controls. Gold-binding fusion protein is indicated in blue, ISBP-free fusion protein in red and the buffer control in green. The y-axis indicates resonance units (RU), the x-axis time in seconds.

FIG. 4 illustrates the association and dissociation of different concentrations of SARS-CoV-2 Spike protein antibody (capture element; anti-S antibody) on a gold sensor coated with gold-binding fusion protein. The y-axis indicates the relative RU response, the x-axis time in seconds.

FIG. 5 is a line graph showing the association and dissociation of various concentrations of Spike protein antigen (S protein) with the immobilized SARS-CoV-2 Spike antibody (anti-S protein antibody). The y-axis indicates the relative RU response, the x-axis time in seconds.

FIG. 6 illustrates the binding of different quantities of gold-binding fusion protein (rows 5-8) versus ISBP-free fusion protein (rows 1-4) to 40 nm gold nanoparticles across a range of pH values.

FIG. 7 is a photograph of a capillary dot blot assay. SARS-CoV-2 Spike protein antigen (Strip 1 and 3) and SARS-CoV-2 Nucleocapsid protein antigen (Strip 2 and 4) were spotted onto nitrocellulose paper strips, which were then dipped into a solution containing gold nanoparticles conjugated with the gold-binding fusion protein and either SARS-CoV-2 Spike protein antibody (Strip 3), SARS-CoV-2 Nucleocapsid protein antibody (Strip 4) or no antibody (Strips 1 and 2).

FIG. 8 is an SPR sensorgram of the immobilization of gold-binding fusion protein (sample) onto a gold sensor surface by direct binding of the gold-fusion protein onto the gold sensor. The y-axis indicates resonance units (RU), the x-axis time in minutes.

FIG. 9 is an SPR sensorgram of the immobilization of gold-binding fusion protein (EMT003) onto a gold sensor surface by NHS-EDC mediated binding of the gold-fusion protein onto the gold sensor. The y-axis indicates resonance units (RU), the x-axis time in minutes.

FIG. 10 is a graph showing the binding of various concentrations (ug/mL) and limit of detection (LoD) of Spike protein antigen (S) and nucleocapsid protein antigen (NC) with the NHS-EDC immobilized EMT003 fusion protein with SARS-CoV-2 Spike antibody or nucleocapsid antibody, respectively (left panel), in comparison to non NHS-immobilized EMT003 fusion protein (right panel). The y-axis indicates the relative RU response, the x-axis antigen concentration in (μg/mL).

FIG. 11A and FIG. 11B are graphs depicting the detection of nucleocapsid antigen (NC) the direct binding EMT003 gold fusion protein-based SPR system in saliva (human pooled) at various concentrations of NC once diluting the saliva in Running Buffer at 1:2; 1:5; 1:10 and 1:20 dilutions. FIG. 11A is the SPR sensorgram detecting binding in real time; FIG. 11B shows the RU response vs dilution of NC in Running Buffer.

FIGS. 12A and 12B show SPR sensorgrams of using EMT003-SARS-CoV-2 Anti-Spike combinations to detect titers of SARS-CoV-2 Spike protein in three different channels, and a control of EMT003-anti-TGFB in a fourth channel. FIG. 12A detects titers of 10-200 ng/mL of SARS-CoV-2 Spike protein, and FIG. 12B detects titers of 300-5,000 ng/mL of SARS-CoV-2 Spike protein in different channels.

FIGS. 13A and 13B illustrates the expression and purity of gold-binding streptavidin fusion proteins on Coomassie-stained SDS-PAGE gels. 2 μg of BSA was added in lane 1 as a loading control. FIG. 13A shows full Gold-binding streptavidin fusion protein EMT027, and FIG. 13B shows full Gold-binding streptavidin fusion protein EMT028.

FIG. 14 is a photograph of a lateral flow assay, showing the detection of antigen immobilized on a strip membrane by EMT027 and EMT028-based conjugates (i.e. gold nanoparticle-streptavidin fusion protein-biotin conjugated detection antibody complex), at different pH of 8.2, 8.7 9.0 and 9.2. for EMT027 and 6.5, 7.0, 7.4 and 7.8 for EMT028.

FIG. 15 is a photograph of a ‘dotted’ sandwich lateral flow assay, showing the detection of dotted nucleocapsid antigen at different concentrations (0.0 μg/ml; 0.001 μg/ml; 0.01 μg/ml; and 0.1 μg/ml), using the EMT028-based gold nanoparticle conjugate loaded with biotin-detection antibody (anti-nucleocapsid) using two different IgG or polyclonal capture antibodies.

FIG. 16 is a photograph of a striped sandwich lateral flow assay, showing the detection of nucleocapsid antigen but not spike antigen using the EMT028-based gold nanoparticle conjugate coupled with nucleocapsid antibody. From left to right: negative control, 1 μg/ml spike antigen, 1 μg/ml nucleocapsid antigen.

FIG. 17 is a photograph of a lateral flow assay, depicting the detection of nucleocapsid antigen at 1 ng/ml and 5 ng/ml in artificial saliva with mucin by the EMT028-based conjugate. In this assay a sample volume of 60 μL of nucleocapsid antigen (at 1 ng/ml or 5 ng/ml) in artificial saliva was applied to each lateral flow strip.

FIG. 18 is an SPR sensorgram screening of nucleocapsid antibody using EMT028 bound to biotinylated nucleocapsid, thereby indicating the detection of antibodies in a screen.

FIG. 19 is a diagram of illustrative embodiments of EMT003, EMT027/EMT028 and GL003 affinity probes.

FIG. 20 is a diagram of illustrative embodiments of a universal dual affinity probe, including a bispecific tandem scFv format (left) and a bispecific immunoglobulin A format (right).

FIGS. 21A-E show Coomassie-stained SDS-PAGE gels indicating the expression and purity of cellulose-binding streptavidin fusion proteins EMT032 and EMT033 (FIGS. 21A-21B), polystyrene-binding streptavidin fusion proteins GL008 and GL009 (FIGS. 21C-21D), and a silica-binding streptavidin fusion protein EMT029 (FIG. 21E).

FIGS. 22A-E show the QCM-D sensor absorption changes for cellulose-binding streptavidin fusion proteins EMT032 and EMT033 (FIGS. 22A-22B), polystyrene-binding streptavidin fusion proteins GL008 and GL009 (FIGS. 22C-22D), and a silica-binding streptavidin fusion protein EMT029 (FIG. 22E).

FIG. 23 shows the diagram of the scFv Troponin fusion (GL007) including the amino acid sequence (SEQ ID NO: 29).

FIG. 24 shows Coomassie-stained SDS-PAGE gels indicating the expression and purity of bispecific antibody GL007.

FIGS. 25A and 25B show the QCM-D sensor absorption changes for GL007 on each sensor and then the addition of troponin antigen (FIG. 25A) and the addition of spike antigen as a control (FIG. 25B).

FIG. 26 shows the purity of the GL011 His-tagged gold-binding streptavidin fusion proteins on a Coomassie-stained SDS-PAGE gel.

FIG. 27 shows the detection by lateral flow assay of Nucleocapsid antigen when diluted in human pooled saliva at 100 ng/mL, 10 ng/mL, and 2 ng/mL and detected by biotinylated detection antibody (SARS-CoV-2 nucleocapsid antibodies) when bound onto streptavidin fusion protein GL011 immobilized on gold nanoparticles.

FIG. 28 is a diagram of illustrative embodiments of a diagnostic lateral flow assay kit, also depicting a method for detecting analyte in a sample by forming a detection complex comprising gold nanoparticles, streptavidin fusion proteins, and biotinylated analyte-bound antibody.

FIG. 29 is a diagram of illustrative embodiments of a diagnostic lateral flow assay kit and a method for detecting analyte in a sample by allowing an analyte to compete for binding to an anti-analyte antibody with a gold nanoparticle-bound antigen. Formation of the antigen-bound gold nanoparticle-streptavidin fusion protein-biotinylated antibody complex is observed on the test line.

FIGS. 30A-B are diagrams of illustrative embodiments of a diagnostic lateral flow assay kit and a method for detecting analyte in a sample by using two compositions in separate lyophilized beads. FIG. 30A shows the first composition comprising antigen-bound gold nanoparticles and the second composition having biotinylated anti-analyte antibody. The biotinylated anti-analyte antibody binds to both the antigen and the provided analyte. FIG. 30B illustrates a method for providing a positive control by binding to the membrane affixed anti-IgY antibody and IgY antibody-coated gold nanoparticles.

FIGS. 31A-B are diagrams of illustrative embodiments of a diagnostic lateral flow assay kit and a method for detecting analyte in a sample by using two analyte compositions comprising two different sets of antibodies. FIG. 31A shows the kit having the first composition in a lyophilized bead that comprises antigen-coated gold nanoparticles and mouse monoclonal anti-analyte antibody. The second composition in another lyophilized bead comprising biotinylated anti-mouse IgG antibody and chicken IgY antibody-coated gold nanoparticles is shown. FIG. 31B shows a method of providing a positive control on the control line by using the membrane affixed anti-chicken IgY antibody and chicken IgY-coated gold nanoparticles.

FIG. 32A-C are diagrams of illustrative embodiments of a diagnostic lateral flow assay kit and a method for detecting analytes in a sample by providing two compositions having either mouse anti-analyte monoclonal antibody or gold nanoparticle-streptavidin fusion protein-biotinylated secondary anti-mouse IgG antibody complexes (FIG. 32A). FIG. 32B and FIG. 32C show the formation of a sandwich complex comprising rabbit IgG antibody, the analyte of interest, mouse IgG antibody, biotinylated secondary antibody, gold nanoparticles, and streptavidin fusion proteins on the test line.

FIG. 33 is a diagram of illustrative embodiments of an exemplary gold detection conjugate and a capture complex. A diagnostic lateral flow sandwich assay and a method for detecting an antigen in a sample by forming a sandwich detection complex is shown.

FIG. 34 is a diagram of illustrative embodiments of a diagnostic lateral flow sandwich assay kit and a method for detecting analytes in a sample by using sandwich biotin-tagged antibody and antibody-conjugated gold nanoparticles.

FIG. 35 is a diagram of illustrative embodiments of a diagnostic lateral flow sandwich assay kit and a method for detecting analyte in a sample by using sandwich His-tagged antibody and antibody-conjugated gold nanoparticles.

FIG. 36 is a diagram of illustrative embodiments of a diagnostic lateral flow sandwich assay kit and a method for detecting analytes in a sample by using anti-analytic antibody fused on gold nanoparticles and another set of anti-analytic antibody grown in a different species. The two sets of secondary antibodies grown in different species are fused to the membrane and form complexes on the test line (analyte bound complex) and the control line (analytic unbound complex).

FIG. 37 is a diagram of illustrative embodiments of a diagnostic lateral flow sandwich assay kit and a method for detecting analyte in a sample by using the assay of FIG. 36 but wherein the gold nanoparticles and dual-affinity probe are encapsulated in lyophilized beads

FIG. 38 includes diagrams of illustrative embodiments, of directly modified antibodies comprising a surface binding moiety fused to the C-terminus of an Fc region of the antibody heavy chain. In particular embodiments, the surface binding moiety binds the surface as a linear peptide, and in other embodiments, the surface binding moiety binds the surface via its 3-D polypeptide structure.

FIG. 39 shows the wet assay results for detecting three concentrations of nucleocapsid antigen when the EMT033 cellulose-binding streptavidin fusion protein is striped on nitrocellulose for capture antibody immobilization

FIG. 40 shows the wet assay results for detecting three concentrations of nucleocapsid antigen when the EMT033 cellulose-binding streptavidin fusion protein is striped on cellulose (Whatman 43 paper) for capture antibody immobilization

FIG. 41 shows the dry lateral flow assay results for detecting whole SARS-CoV-2 virus when the EMT033 cellulose-binding streptavidin fusion protein was striped on cellulose (Whatman 43 paper) for capture antibody immobilization

FIG. 42 shows the dry lateral flow assay results for detection of SARS-CoV-2 nucleocapsid antigen when either the capture antibody is immobilized by binding to a cellulose-binding streptavidin fusion protein or physiosorbed with no fusion protein.

FIGS. 43A-C show the QCM-D sensor absorption changes for various PDMS binding fusion motifs, GL014 (FIG. 43A); GL015 (FIG. 43B); and GL016 (FIG. 43C) and the addition of biotinylated SARS-CoV-2 nucleocapsid antibody and SARS-CoV-2 nucleocapsid antigen.

### DETAILED DESCRIPTION

The following terms are defined below.

As used herein, the term “antibody” means an isolated or recombinant binding agent that comprises the necessary variable region sequences to specifically bind an antigenic epitope. Thus, the term “antibody” includes antibodies and antigen-binding fragments thereof, and an antibody is any form of antibody or fragment thereof that exhibits the desired biological activity, e.g., binding the specific target antigen. Thus, it is used in the broadest sense and specifically covers monoclonal antibodies (including full-length monoclonal antibodies), polyclonal antibodies, human antibodies, humanized antibodies, chimeric antibodies, camelid antibodies (or VHH), sdAB (nanobodies), diabodies, multispecific antibodies (e.g., bispecific antibodies), and antibody fragments including but not limited to scFv, Fab, and Fab2, so long as they exhibit the desired biological activity.

A single-domain antibody (sdAb), also known as a nanobody, is an antibody fragment consisting of a single monomeric variable antibody domain. Like a whole antibody, it is able to bind selectively to a specific antigen. sdAb with a molecular weight of only 12-15 kDa are much smaller than common full antibodies (about 150-160 kDa), which are composed of two heavy protein chains and two light chains, and even smaller than Fab fragments (˜50 kDa, one light chain and half a heavy chain) and single-chain variable fragments (˜25 kDa, two variable domains, one from a light and one from a heavy chain). The first single-domain antibodies were engineered from heavy-chain antibodies found in camelids; these are also called V_{H}H fragments.

“Antibody fragments” comprise a portion of an intact antibody, for example, the antigen-binding or variable region of the intact antibody. Examples of antibody fragments include Fab, Fab′, F(ab′)2, and Fv fragments; diabodies; linear antibodies (e.g., Zapata et al., Protein Eng. 8(10): 1057-1062 (1995)); single-chain antibody molecules (e.g., scFv); and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)2 fragment that has two antigen combining sites and is still capable of cross-linking antigen.

The term “antigen” refers to a molecule or a portion of a molecule capable of being bound by a selective binding agent, such as an antibody, and additionally capable of being used in an animal to produce antibodies capable of binding to an epitope of that antigen. In certain embodiments, a binding agent (e.g., a capture element of a dual affinity probe) is said to specifically bind an antigen when it preferentially recognizes its target antigen in a complex mixture of proteins and/or macromolecules. In a specific embodiment, the antigen is not obtained in the sample for detection. In another embodiment, the antigen is a recombinant antigen or is exogenous from the sample, and is instead provided in the testing system or diagnostic kit. In another embodiment, the antigen can be provided in the testing system or diagnostic kit to be a competitive protein or peptide to the analyte.

The term “analyte” as used herein refers to the protein, peptide, antibody, chemical entity, or target, such as from a sample, that is to be identified or measured. Non-limiting examples of analytes include: pathogens, proteins, peptides, antibodies, chemical entities, hormones, environmental toxins, steroids, etc. In a specific embodiment, the sample is from a subject, such as a human.

The term “antigen-binding fragment” as used herein refers to a polypeptide fragment that contains at least one CDR of an immunoglobulin heavy and/or light chain, or of a Nanobody® (Nab), that binds to the antigen of interest, e.g., a pathogen. In this regard, an antigen-binding fragment of the herein described antibodies may comprise 1, 2, 3, 4, 5, or all 6 CDRs of a VH and VL from antibodies that bind one or more analyte, e.g., pathogen.

Aptamers may be comprised of protein and/or nucleic acid. Peptide aptamers are artificial proteins selected or engineered to bind specific target molecules. These proteins consist of one or more peptide loops of variable sequence displayed by a protein scaffold. They are typically isolated from combinatorial libraries and often subsequently improved by directed mutation or rounds of variable region mutagenesis and selection. In vivo, peptide aptamers can bind cellular protein targets and exert biological effects, including interference with the normal protein interactions of their targeted molecules with other proteins. Nucleic acid aptamers are nucleic acid species that exhibit binding affinity for a given target with selectivity and specificity comparable to antibodies. Nucleic acid aptamers can consist of either RNA or DNA. Nucleic acid aptamers may be generated via in-vitro selection methods such as SELEX (systematic evolution of ligands by exponential enrichment) to express affinity for targets ranging from small ligands such as heavy metal ions and small molecules, up to larger ligands such as proteins and cells. The aptamer-ligand interaction is mediated through non-covalent forces; electrostatic interactions, hydrophobic interactions, pi-pi orbital stacking, and hydrogen bonding interactions. Aptamers can be engineered completely in vitro, are readily produced by chemical synthesis, possess desirable storage properties, and elicit little or no immunogenicity in therapeutic applications. X-Aptamers are a new generation of aptamers designed to improve on the binding and versatility of regular DNA/RNA-based aptamers. X-Aptamers are engineered with a combination of natural and chemically-modified DNA or RNA nucleotides. Base modifications allow incorporation of various functional groups/small molecules into X-aptamers, opening a wide range of uses and a higher likelihood of binding success compared to standard aptamers. Thiophosphate backbone modifications at selected positions enhance nuclease stability and binding affinity without sacrificing specificity. Optimers also include other forms, such as split optimers and optimer ligands.

The term “a linker sequence” is intended to mean a sequence that bridges the surface binding entity, e.g., inorganic surface binding entity, with the organic binding entity, e.g., capture element. As used herein, a linker sequence may comprise one or both of an active linker and/or a passive linker. Thus, a linker sequence may, for example, comprise the amino acid sequence of protein G from Streptococcus or streptavidin from Streptomyces, or may be a simple amino acid sequence or simply a single bond, such as a covalent bond. Organic binding entities include both synthetic carbon-based compounds as well as biologically-derived molecules.

The term “surface binding motif”, “surface binding moiety”, or SBM is intended to mean a molecule with specific and selective affinity for an organic or inorganic substance, such as, e.g., polydimethylsiloxane (PDMS), zinc oxide (ZnO), gold, quartz, silica, silicon, silver, cellulose, nitrocellulose, plastic, polystyrene and graphene. An SBM may be a peptide or polypeptide, and may include additional components such as a NACE fused or bound to the SBM. The term “inorganic surface binding peptides” or ISBP is intended to mean a sequence of amino acids with specific and selective affinity for an inorganic substance such as gold, silica or graphene. The ISBP may thus, for example, include a short peptide, a protein, an antibody with an affinity to the inorganic surface or fragment of an antibody, a camelid antibody, a modified antibody, a single chain variable fragment, or a Fab fragment, and may include additional components such as a NACE fused or bound to the ISBP. The ISBP may thus, for example, include a short peptide, a protein, an antibody with an affinity to the inorganic surface or fragment of an antibody, a camelid antibody, a modified antibody, a single chain variable fragment, or a Fab fragment, and may include additional components such as a NACE fused or bound to the ISBP. The term “surface binding peptides” or SBP is intended to mean a sequence of amino acids with specific and selective affinity for a substance or surface, such as polydimethylsiloxane (PDMS), zinc oxide (ZnO), gold, quartz, silica, silicon, silver, cellulose, nitrocellulose, plastic, polystyrene and graphene.

The term “biosensor” is intended to mean a component or device that converts the detection of an analyte, e.g., a pathogen, into a measurable signal using biological components. The term “biosensor material” is intended to mean something that converts biological or chemical reactions into measurable signals that are proportional to an analyte, e.g., a pathogen, of interest. The signal generated can be in the form of heat, light, pH, mass or charge change, for example.

The term “capture element” is intended to include any moiety such as antigen, biotin, protein G from Streptococcus or streptavidin from Streptomyces or a single chain variable fragment or an antibody, or binding fragment thereof, optionally wherein the antibody is a non-fragmented antibody, a modified antibody, a camelid antibody, a single chain variable fragment, or a Fab fragment, for example SARS-CoV-2 Spike and SARS-CoV-2 Nucleocapsid targeting antibody is capable of binding to the analyte or target being detected and/or quantified. In a specific example, the analyte is from the sample for detection or quantification.

The term “non-analyte capture element” or NACE, is intended herein to include any moiety such as antigen, biotin, protein G from Streptococcus or streptavidin from Streptomyces or a single chain variable fragment or an antibody, or binding fragment thereof, optionally wherein the antibody is a non-fragmented antibody, a modified antibody, a camelid antibody, a single chain variable fragment, or a Fab fragment, but does not specifically bind to the analyte, but instead a non-analyte moiety or target molecule. For example, the non-analyte capture element can specifically bind to another moiety in the dual-affinity probe or another moiety on the surface of a system or diagnostic kit.

The term “covalent fusion” is intended to mean the joining of two or more genes that encode separate peptides or proteins. The terms “polypeptide” “protein” and “peptide” are used interchangeably and mean a polymer of amino acids not limited to any particular length. The term does not exclude modifications such as myristylation, sulfation, glycosylation, phosphorylation and addition or deletion of signal sequences. The terms “polypeptide” or “protein” or “peptide” means one or more chains of amino acids, wherein each chain comprises amino acids covalently linked by peptide bonds, and wherein said polypeptide or protein or peptide can comprise a plurality of chains non-covalently and/or covalently linked together by peptide bonds, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. Thus, a “polypeptide” or a “protein” can comprise one (termed “a monomer”) or a plurality (termed “a multimer”) of amino acid chains.

The term “fusion protein” means a protein comprised of at least two different amino acid sequences and generated within an organism such as E. coli or insect cells of Spodoptera frugiperda. An inorganic surface binding peptide expressed with A or G protein or a linker is an example of a fusion protein.

“Pathogens” include pathogenic agents that cause mammalian infection or disease, including, e.g., viruses, bacteria, etc., such as any of those disclosed herein, including but not limited to: SARS-CoV-2, influenza viruses, Adenovirus, CMV, Coxsackievirus, Dengue Virus, Epstein Barr virus (EBV), Enterovirus 71 (EV71), Ebola Virus, Hepatitis A virus (HAV), Hepatitis B virus (HBV), Human cytomegalovirus (HCMV), Hepatitis C virus (HCV), Hepatitis D virus (HDV), Hepatitis E virus (HEV), Human Immunodeficiency Virus (HIV), Human papilloma virus (HPV), Herpes simplex virus (HSV), Human T-lymphotropic virus (HTLV), Influenza A Virus, Influenza B Virus, Japanese Encephalitis, Leukemia Virus, and Ebola Virus, Measles Virus, Molluscum Contagiosum, Orf Virus, Parvovirus, Rabies Virus, Respiratory Syncytial Virus, Rift Valley Fever Virus, Rubella Virus, Rotavirus, Varicella Zoster Virus, Variola, West Nile Virus, Zika Virus, and Chikungunya Virus. The term “pathogen” is also intended to include proteins or peptides of a pathogen, including but not limited to proteins or peptides that indicate the presence of a disease-causing organism or virus, and/or biomarkers for a disease-causing organism or virus, for example spike and nucleocapsid proteins of human coronaviruses, including SARS-CoV-2, influenza hemagglutinin, antigens of Adenovirus, CMV, Coxsackievirus, Dengue Virus, EBV, EV71, Ebola Virus, HAV, HBV, HCMV, HCV, HDV, HEV, HIV, HPV, HSV, HTLV, Influenza A Virus, Influenza B Virus, Japanese Encephalitis, Leukemia Virus, Measles Virus, Molluscum Contagiosum, Orf Virus, Parvovirus, Rabies Virus, Respiratory Syncytial Virus, Rift Valley Fever Virus, Rubella Virus, Rotavirus, Varicella Zoster Virus, Variola, West Nile Virus, Zika Virus, and Chikungunya Virus.

The term “specifically binds” means that a molecule reacts or associates more frequently, more rapidly, with greater duration and/or with greater affinity with a particular target molecule, e.g., a pathogen, than it does with alternative molecules, e.g., pathogens. It is also understood by reading this definition that a molecule that specifically or preferentially binds to a first target may or may not specifically or preferentially bind to a second target. As such, “specific binding” does not necessarily require (although it can include) exclusive binding.

With respect to antibodies, KD is the equilibrium dissociation constant, a calculated ratio of Koff/Kon, between the antibody and its antigen. The association constant (Kon) is used to characterise how quickly the antibody binds to its target. The dissociation constant (Koff) is used to measure how quickly an antibody dissociates from its target. KD and affinity are inversely related. A high affinity interaction is characterized by a low KD, a fast recognizing (high Kon) and a strong stability of formed complexes (low Koff). In certain embodiments, a dual affinity probe, or the capture element thereof binds to its target with a KD of at least or less than 1×10⁻², at least or less than 1×10⁻³, at least or less than 1×10⁻⁴, at least or less than 1×10⁻⁵, at least or less than 1×10⁻⁶, at least or less than 1×10⁻⁷, at least or less than 1×10⁻⁸, at least or less than 1×10⁻⁹, at least or less than 1×10⁻¹⁰, at least or less than 1×10⁻¹¹, or at least or less than 1×10⁻¹². For purposes of this invention, KD is determined from a binding curve using a Biacore2000 measuring device according to the analysis software provided with the device.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

In this disclosure, the word “comprising” is used in a non-limiting sense to mean that items following the word are included, but items not specifically mentioned are not excluded.

It will be understood that in embodiments which comprise or may comprise a specified feature or variable or parameter, alternative embodiments may consist, or consist essentially of such features, or variables or parameters. A reference to an element by the indefinite article “a” does not exclude the possibility that more than one of the elements is present, unless the context clearly requires that there be one and only one of the elements.

In this disclosure the recitation of numerical ranges by endpoints includes all numbers subsumed within that range including all whole numbers, all integers and all fractional intermediates (e.g., 1 to 5 includes 1, 1.5, 2, 2.75, 3, 3.80, 4, and 5, etc). In this disclosure the singular forms an “an”, and “the” include plural referents unless the content clearly dictates otherwise. Thus, for example, reference to a composition containing “a compound” includes a mixture of two or more compounds.

In this disclosure term “or” is generally employed in its sense including “and/or” unless the content clearly dictates otherwise.

The disclosure provides certain polypeptide sequences. The skilled artisan will appreciate that these particular sequences may be modified and still achieve the same result, e.g., binding to another polypeptide, to a surface or to an analyte. Thus, the disclosure includes variants of any of the disclosed polypeptides, and all uses thereof. In particular embodiments, a polypeptide variant or comprises one or more amino acid modifications, respectively, as compared to the disclosed sequence. Modifications include but are not limited to amino acid substitutions, insertions, and deletions. Variant polypeptides may comprise at least 80%, at least 85%, at least 90%, at least 95%, at least 98%, or at least 99% sequence identity to a sequence disclosed herein. As used herein, the terms “identity” and “identical” refer, with respect to a polypeptide sequence, to the percentage of exact matching residues in an alignment of that “query” sequence to a “subject” sequence, such as an alignment generated by the BLAST algorithm. Identity is calculated, unless specified otherwise, across the full length of the subject sequence. Thus a query sequence “shares at least x % identity to” a subject sequence if, when the query sequence is aligned to the subject sequence, at least x % (rounded down) of the residues in the subject sequence are aligned as an exact match to a corresponding residue in the query sequence. Where the subject sequence has variable positions (e.g., residues denoted X), an alignment to any residue in the query sequence is counted as a match.

The disclosure provides compositions and methods for detecting the presence and or quantity of an analyte in a test sample. The disclosure may include elements as described in PCT application PCT/CA2021/051256, which is incorporated by reference herein in its entirety.

Aspects of the disclosure related to dual-affinity probes, or specifically dual-affinity immunoprobes that may be used to determine the presence or absence or an analyte in a test sample, wherein the dual-affinity probes comprise an inorganic surface binding polypeptide and an analyte-specific capture element.

### Dual Affinity Probes

In a specific embodiment, the compositions may comprise a dual-affinity probe, which may be use for detecting an analyte, e.g., an infectious agent or pathogen, in a sample, the dual-affinity probe comprising a surface binding motif (SBM), e.g., an inorganic surface binding peptide (ISBP), and a capture element (CE). In another embodiment, the dual-affinity probe may be a dual-affinity immunoprobe, meaning the probe may be utilized with the use of an antibody or antibody fragment. For example, the SBM and/or the CE may comprise an antibody or an antigen-binding fragment thereof.

In a specific embodiment, the CE may be a molecularly imprinted polymer (MIP), an aptamer, a nanobody, or an engineered protein.

In certain embodiments of dual-affinity probes, the SBM or ISBP is a peptide. In particular embodiments, the SBM or ISBP is an antibody, or an antigen-binding fragment thereof, e.g., an scFv. A variety of surface binding peptides are known in the art, and illustrative surface binding peptides are disclosed herein.

In particular embodiments, the analyte is a pathogen, and the analyte-specific capture element specifically binds to the pathogen. In certain embodiments, the analyte-specific capture element is an antibody, or an antigen binding fragment thereof, e.g., an scFv. Antibodies that specifically bind to various pathogens, including but not limited to those disclosed herein, are known in the art, and may be readily produced.

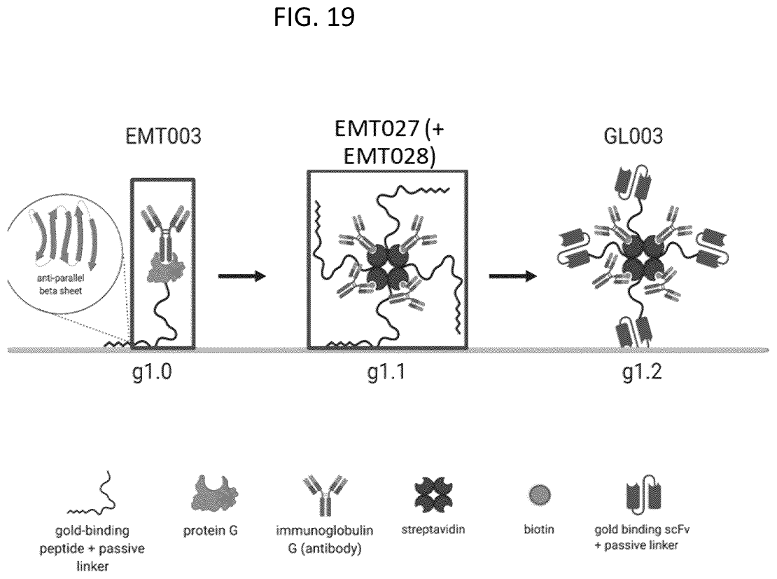

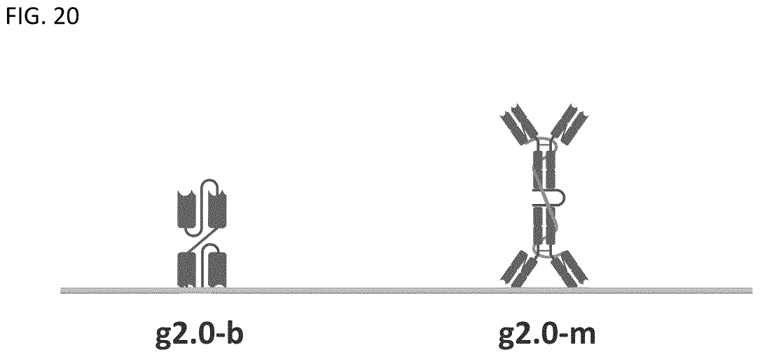

The disclosure contemplates various formats of dual-affinity probes. In certain embodiments, the dual-affinity probe comprises one or more polypeptide that binds to both a specific surface and one or more specific target analyte. In other embodiments, the dual-affinity probe comprises two or more polypeptides, including a first polypeptide that binds to a specific surface and also includes an active linker that binds to a class of molecules, such as antibodies, or to a specific member of a binding pair, such as streptavidin/biotin; and a second polypeptide comprising a target specific capture element, wherein the second polypeptide is bound by the active linker. For example, the second polypeptide may comprises an antibody, or antigen-binding fragment thereof, that specifically binds the target analyte, and/or it may comprise a member of a binding pair that is bound by the other member of the binding pair present in the first polypeptide. Thus, while certain dual-affinity probes specifically bind one or more target analytes, e.g., pathogens, other dual-affinity probes may be adapted to identity any of a variety of different target analytes, depending on the nature of the capture element, i.e., the target analyte it binds. Diagrams of various illustrative configurations of dual-affinity probes are provided in FIGS. 19 and 20.

In particular embodiments, the SBM and the CE are present within the same polypeptide, and may be directly fused to each other or fused to each other via one or more linker, e.g., a passive linker, such as a bond or a glycine-serine linker, or an IgA J chain or a llama IgG hinge region. In particular embodiments, the analyte-specific capture element specifically binds to an analyte of interest. In certain embodiments, the analyte-specific capture element is an antibody or an antigen-binding fragment thereof, e.g., such as an scFv. In certain embodiments, the dual-affinity probe is a single fusion protein. In another embodiment, the CE and ISBP is independently an antibody, a fragment of an antibody, or a single chain variable fragment from an antibody. In another embodiment, the ISBP is a single chain variable fragment from an antibody. In another embodiment, the single chain variable fragment is a V_{H} binding motif. In a specific embodiment, the V_{H} binding motif is a gold V_{H} binding motif. In another embodiment, the CE is a single chain variable fragment from an antibody. In a specific embodiment, the ISBP and CE are fused as a bispecific antibody fragment.

In particular embodiments, the SBM and the CE are present in different polypeptides. For example, in certain embodiments, the dual-affinity probes comprise a first polypeptide comprising the SBM and an active linker, and a second polypeptide comprising the CE, wherein the active linker is capable of binding to the second polypeptide comprising the analyte-specific capture element. In certain embodiments, the active linker directly binds the analyte specific capture element; for example, the active linker may be protein A, protein G, or anti-IgG (e.g., goat anti-human IgG), and the analyte specific capture element may be an antibody, or antigen binding fragment thereof. In other embodiments, the analyte specific capture element is fused to a non-specific binding element that directly binds to the active linker; for example, the non-specific capture element may be biotin, and the active linker may be streptavidin, or vice versa. In certain embodiments, protein G is fused to the N-terminus or the C-terminus of the SBM, e.g., via a passive linker, such as a peptide linker. In certain embodiments, streptavidin is fused to the N-terminus or the C-terminus of the SBM, e.g., via a passive linker, such as a peptide linker. Various other binding pairs, in addition to biotin and streptavidin are known in the art, and could alternatively be used.

In certain embodiments, the capture element (CE) is connected to the SBM via a linker sequence (LI), wherein LI may be a single bond or an amino acid sequence, and the linker sequence is further connected to the SBM, e.g., an ISBP. In particular embodiments, the linker (LS) comprises one or more passive linker (PL) and/or one or more active linker (AL). The dual-affinity probe may have the following formula (I) or formula (II): SBM-LI-CE (I) or CE-LI-SBM (II).

In certain embodiments, the dual-affinity probe comprises at least two polypeptides, including a first polypeptide of formula (IIIa) or (IIIb), wherein PL is a passive linker, such as a single bond or passive peptide linker, and AL is an active linker that binds to the polypeptide of formula IV(a) or (IVb), wherein active linker binder (ALB) is a polypeptide sequence bound by the AL, wherein LI is a passive linker, such as a single bond or passive peptide linker, and wherein ALB and AL may be absent or present:

SBM-PL-AL (IIIa)

AL-PL-SBM (IIIb)

ALB-PL-CE (IVa)

CE-PL-ALB (IVb).

In a specific embodiment, formula (I) or formula (II), (IIIa), (IIIb), (IVa), (IVb) or any other formula described herein, each formula can start at the N-terminal end or the C-terminal end of the protein or peptide. As matter of illustration, formula IIIA SBM-PL-AL (IIIa) allows for the SBM at the N-terminal end (N-terminal end orientation) and formula IIIb allows for SBM at the C-terminal end (C-terminal end orientation). Each specific formula and embodiment, described herein includes and allows for both the N-terminal end orientation and the C-terminal end orientation.

In a specific embodiment, the SBM or ISBP is connected to an inorganic surface, which may include an inorganic surface of a biosensor or other biosensor material. The inorganic surface or biosensor material that the SBM or ISBP may be connected to may include, e.g., polydimethylsiloxane (PDMS), zinc oxide (ZnO), gold, silica, silver, cellulose, plastic, polystyrene and graphene. In a specific embodiment, the biosensor material is selected from the group consisting of gold, cellulose, silica and polystyrene.

The dual-affinity probes may use such materials in various forms of biosensors or diagnostic platforms. For example, the biosensors or platforms may use technologies such as quartz crystal microbalance, surface plasmon resonance (SPR) or by a lateral flow assay.

The dual-affinity probes may incorporate any SBM or ISBP or LI or CE in any combination as described herein.

In other embodiment, the dual affinity probes (DAP) may be used for detecting an analyte in a sample, and may include: i) a surface binding moiety (SBM) fused or bound to a non-analyte capture element (NACE), or ii) a surface particle fused or bound to a NACE or a capture element (CE).

In a specific embodiment, the surface particle can be any surface, including for example polydimethylsiloxane (PDMS), zinc oxide (ZnO), gold, quartz, silica, silicon, silver, cellulose, nitrocellulose, plastic, polystyrene and graphene. In a specific embodiment, the surface particle is gold.

In another embodiment, the particles may be in the form of beads, and may be lyophilized. In a specific embodiment, the DAP may include an SBM with a surface binding peptide (SBP) that specifically binds to the surface particle. In a specific embodiment, the SBP is gold binding protein or peptide.

In another embodiment, the NACE is one or more selected from the group consisting of a linker (LI), streptavidin, protein G, biotin, an antigen protein or peptide, or an antibody, or binding fragment thereof, wherein the antibody is a non-fragmented antibody, a modified antibody, a single chain variable fragment from an antibody, or a Fab fragment, and wherein the CE is an antibody or binding fragment thereof selected from the group consisting of: a non-fragmented antibody, a modified antibody, a camelid antibody, a single chain variable fragment, a Fab fragment, and an antigen protein or peptide.

In a specific embodiment, the DAP may have a specific order of binding, such as wherein a surface particle is bound to a surface binding peptide (SBP), wherein the SBP is fused or bound to a non-analyte capture element (NACE), wherein the NACE is additionally fused or bound to a CE and the DAP has the following Formula Ic or Formula IIc:

(Surface particle)-SBP-NACE-CE (Formula Ic); or

(Surface particle)-SBP-CE (Formula IIc).

In another embodiment, the NACE is one or more selected from the group consisting of a linker (LI), streptavidin, protein G, biotin, an antigen protein or peptide, or an antibody, or binding fragment thereof, wherein the antibody is a non-fragmented antibody, a modified antibody, a single chain variable fragment from an antibody, or a Fab fragment, and wherein the CE is an antibody or binding fragment thereof selected from the group consisting of: a non-fragmented antibody, a modified antibody, a camelid antibody, a single chain variable fragment, a Fab fragment, and an antigen protein or peptide.

In one embodiment, the SBP comprises a nitrocellulose binding protein/peptide, a silicon binding protein/peptide, a cellulose binding motif, a polystyrene binding motif, and/or a silica binding motif. In another embodiment, the SBP comprises a sequence from Table 1 herein.

In a specific embodiment, the NACE comprises one or more selected from the group consisting of a linker, streptavidin, protein G, biotin, biotinylated protein, an antigen protein or peptide, and an antibody, or binding fragment thereof, optionally wherein the antibody is a non-fragmented antibody, a modified antibody, a camelid antibody, a single chain variable fragment, or a Fab fragment. In a specific embodiment, the NACE comprises an antibody or binding fragment thereof, optionally a non-fragmented antibody, a modified antibody, a camelid antibody, a single chain variable fragment, or a Fab fragment, that specifically binds to IgG, e.g., an IgG1, IgG2, IgG3, an IgG4, IgM, IgD, and IgE, but preferably an IgG. In another embodiment, the NACE comprises protein G and/or streptavidin. In another embodiment, the NACE comprises a binding tag, such as a His tag or biotin tag. In another embodiment, the NACE comprises a cleave site, such as a TEV cleavage site, or similar such sequences are sites known in the art.

In another embodiment, the CE is an antibody or binding fragment thereof, optionally wherein the antibody or binding fragment thereof is selected from the group consisting of: a non-fragmented antibody, a camelid antibody, a single chain variable fragment from an antibody, and a Fab fragment, or an antigen protein or peptide. In another embodiment, the CE is a non-fragmented antibody. In another specific embodiment, the antibody comprises a tag sequence, optionally a biotin tag or a his-tag.

In a specific embodiment, the surface particle can be any surface, including for example polydimethylsiloxane (PDMS), zinc oxide (ZnO), gold, quartz, silica, silicon, silver, cellulose, nitrocellulose, plastic, polystyrene and graphene. In a specific embodiment, the surface particle is gold.

In another embodiment, the particles may be in the form of beads, such as analyte beads for binding specifically to the analyte. In a specific embodiment, the analyte beads are lyophilized. In another specific embodiment, the lyophilized surface particles or analyte beads range in the size of about 10 nm to about 80 nm. In another specific embodiment, the lyophilized surface particles or analyte beads range in the size of about 15 nm to about 60 nm. In another specific embodiment, the lyophilized surface particles or analyte beads range in the size of about 30 nm to about 50 nm.

In a specific embodiment, the dual-affinity probe may be utilized for binding a gold surface. In a specific embodiment, the gold surface may in the form of particles, such as nanoparticles, and/or beads. In another embodiment, the gold surface may be bound to a gold surface peptide/protein.

In a specific embodiment, the probe may include i) a surface binding moiety (SBM), wherein the SBM is a gold binding protein (GBP), fused or bound to a non-analyte capture element (NACE), or ii) a surface binding moiety (SBM), wherein the SBM is a gold binding protein (GBP), fused or bound to a capture element (CE).

In another specific embodiment, the gold particle of the probe is bound to the gold binding protein (GBP), and the GBP is fused or bound to the CE (Formula IIIc) or the NACE, wherein the NACE is additionally fused or bound to a CE (Formula IVc) as provided below:

(Gold particle)-GBP-CE (Formula IIc); or

(Gold particle)-GBP-NACE-CE (Formula IVc).

In another specific embodiment, NACE comprises one or more selected from the group consisting of a linker, streptavidin, protein G, biotin, biotinylated protein, an antigen protein or peptide, and an antibody or binding fragment thereof, optionally wherein the antibody is a non-fragmented antibody, a modified antibody, a camelid antibody, a single chain variable fragment, or a Fab fragment. In another embodiment, the NACE comprises an antibody or binding fragment thereof, optionally a non-fragmented antibody, a modified antibody, a camelid antibody, a single chain variable fragment, or a Fab fragment, that specifically binds to IgG.

In another embodiment, the CE is an antibody or binding fragment thereof, optionally wherein the antibody or binding fragment thereof is selected from the group consisting of: a non-fragmented antibody, a camelid antibody, a single chain variable fragment from an antibody, and a Fab fragment, or an antigen protein or peptide. In a specific embodiment, the CE is a non-fragmented antibody. In another specific embodiment, the CE comprises a tag sequence, such as a biotin tag or a his-tag.

In a specific embodiment, a fusion protein may comprise a sequence from Table 1, Table 1a, Table 6, Table 7, Table 10, and Table 12. In a specific embodiment, the fusion protein may comprise the amino acid sequence from Table 1a. In a specific embodiment, the fusion protein may consist of a sequence from Table 1, Table 1a, Table 6, Table 7, Table 10, and Table 12. In a specific embodiment, the fusion protein may consist of a sequence from Table 1a. In another specific embodiment, the fusion protein may comprise sequences from Table 1, Table 1a, Table 6, Table 7, Table 10, and Table 12 with at least 80%, at least 85%, at least 90%, at least 95%, at least 98%, or at least 99% sequence identity. In a specific embodiment, the fusion protein may comprise of a sequence from Table 1a with at least 80%, at least 85%, at least 90%, at least 95%, at least 98%, or at least 99% sequence identity.

In a specific embodiment, the GBP may include a sequence such as EMT014, EMT015, EMT016, EMT017, EMT018, and EMT019.

In a specific embodiment, the GBP is fused or bound to an additional NACE compound selected from the group consisting of protein G, streptavidin, biotin, and biotinylated protein. In another embodiment, the NACE comprises protein G and the protein G is fused to the gold binding protein. In another specific embodiment, the NACE comprises streptavidin and the streptavidin is bound to the GBP.

In a specific embodiment, the GBP may separately or as a fusion protein, include a sequence from Table 1 or Table 1a, such as EMT014, EMT015, EMT016, EMT017, EMT018, EMT019, EMT020, EMT021, EMT022, EMT023, EMT024, EMT003, EMT027, EMT028 and GL011.

In a specific embodiment, the GBP-NACE is a fusion protein selected from GL011, GL017, GBP-protein G fusion (EMT03), and a GBP streptavidin fusion EMT027, or EMT028.

In another embodiment, the NACE additionally comprises an antibody, or a Fab fragment that is specific for binding to IgG, or biotinylated antibody.

In another embodiment, the gold particles may be in the form of beads, such as analyte beads for binding specifically to the analyte. In a specific embodiment, the analyte beads are lyophilized. In another specific embodiment, the lyophilized surface particles or analyte beads range in the size of about 10 nm to about 80 nm. In another specific embodiment, the lyophilized gold surface particles or analyte beads range in the size of about 15 nm to about 60 nm. In another specific embodiment, the lyophilized gold surface particles or analyte beads range in the size of about 30 nm to about 50 nm.

Capture Element (CE) of Dual-Affinity Probe

The capture element (CE) of the present invention may include any organic binding entity that binds to a specific analyte of interest. In particular embodiments, the analyte is an infectious agent or pathogen, and the analyte-specific capture element specifically binds to the infectious agent or pathogen. In certain embodiments, the analyte-specific capture element is an antibody, or an antigen binding fragment thereof, e.g., an scFv. Antibodies that specifically bind to various infectious agents and pathogens, including but not limited to those disclosed herein, are known in the art, and may be readily produced. In a specific embodiment, the capture element is a fragment of an antibody such as a single chain variable fragment, or a Fab fragment.

The capture element may also be an amino acid sequence that is not an antibody or antibody fragment, but any amino acid sequence, peptide, protein or specific antigen that binds to the analyte. In certain embodiments, the capture element is an aptamer or ligand. In certain embodiments, the capture element is an antibody, e.g., an scFv or a nanobody. In certain embodiments, the methods disclosed herein may be used to determine the presence and/or amount of antibodies that bind to an infectious agent or pathogen, including but not limited to any of those disclosed herein, present in a sample, e.g., a biological sample. In certain embodiments, the capture element may be applied to test the sample of the subject to determine if the subject has antibodies for a specific pathogen or infectious agent, and more specifically a specific antigen or epitope thereof that identifies the pathogen. Thus, in a specific embodiment, the capture element comprises at least a portion of an antigen, or epitope thereof, bound by one or more antibodies that specifically bind the pathogen. In certain embodiments, the antigen may be any agent capable of inducing an immune response, e.g., in a mammal, that results in the product of antibodies that bind the antigen.

The capture element may be specific to any analyte or pathogen of interest, for example, the capture element may be specific to an antigen, protein, peptide, or other organic element that identifies that a subject may be positive for or infected with a specific pathogen. In a specific embodiment the capture element is specific to an antigen for SARS-CoV-2. In another specific embodiment, the capture element is specific for SARS-CoV-2 Spike (S) antigen or SARS-CoV-2 Nucleocapsid (N) antigen. In a specific embodiment, the capture element is an antibody and is an S or N antigen targeting antibody specific for SARS-CoV-2 Spike (S) antigen or SARS-CoV-2 Nucleocapsid (N) antigen. In another specific embodiment, the antibodies may be the specific antibodies listed in Table 2 herein.

Other pathogens that the capture element may be specific for include, but are not limited to, Coronavirus spp. Such as SARS and MERS; Influenza spp.; Respiratory Syncytial Virus spp.; Adenovirus spp.; Parainfluenza spp.; Filoviridae such as Ebola and Marburg; Hantavirus spp.; Arenaviridae such as Lassa; Bunyaviridae such as Rift Valley and Crimean-Congo; and Paramyxoviridae such as Hendra and Nipah; for example. Pathogens include, in some embodiments, prions. Pathogens include, in some embodiments, Gram negative and Gram positive bacteria. Other pathogens may include for example infectious diseases. The capture element for example may be specific to an analyte or antigen in infectious diseases such as hepatitis B & C, HIV, syphilis, chlamydia and gonorrhea.

In another embodiment, the capture element is an antigen and is specific to unique pathogen such as SARS-CoV-2. In a specific embodiment, the antigen comprises at least a portion of the spike protein of SARS-CoV-2. In another embodiment, the antigen comprises at least the full sequence of the spike protein or any variants thereof.

In another specific embodiment, the capture element (CE) is an antigen that is fused or bound to the dual affinity probe. In another specific embodiment, the CE is an antigen fused to a linker or SBM/ISBP. In another specific embodiment, the CE antigen is biotinylated and binds to a streptavidin linker. In another specific embodiment, the CE is an antigen that binds to an antibody (or antibodies), the intended analyte for detection. In a specific embodiment, the antigen protein is SARS-CoV-2 Spike and/or SARS-CoV-2 Nucleocapsid proteins. In another specific embodiment, the antigen binds to and detects antibodies. In another embodiment, the antibody or antibodies are a targeting antibody specific for SARS-CoV-2 Spike (S) antigen or SARS-CoV-2 Nucleocapsid (N) antigen, or an antigen-binding fragment thereof.

In a specific embodiment, the capture element may be linked to the linker (LI) or ISBP to ensure that there is effective binding to the analyte of interest. For example, the spike protein of SARS-CoV-2 may be linked to the linker (LI) or ISBP to ensure that the correct portion of the protein or epitope is exposed to the analyte, and in this case antibodies that would be specific to various portions of the spike protein. Methods for attaching a capture element or specific amino acid sequence to another amino acid sequence are known in the art, and may be applied in the specific invention described herein. For example, in another embodiment the capture element may be tagged or modified for the purpose of binding specifically to a linker or directly to the ISBP. For example, the capture element may be biotinylated solely for binding to a streptavidin linker, such as streptavidin from Streptomyces. In another embodiment, the capture element may be an antibody or an element that is modified to more efficiently bind to a linker such as protein G, which is specific to IgG and protein G from Streptococcus.

Non-Analyte Capture Element of the Dual-Affinity Probe

Non-analyte capture elements (NACE) may include any binding moiety such as antigen, biotin, protein G from Streptococcus or streptavidin from Streptomyces or a single chain variable fragment or an antibody, or binding fragment thereof, optionally wherein the antibody is a non-fragmented antibody, a modified antibody, a camelid antibody, a single chain variable fragment, or a Fab fragment, but does not specifically bind to the analyte, but instead a non-analyte moiety or target molecule.

The non-analyte capture element can specifically bind to another moiety in the dual-affinity probe or another moiety on the surface of a system or diagnostic kit.

In one embodiment, NACE comprises one or more selected from the group consisting of a linker, streptavidin, protein G, biotin, biotinylated protein, an antigen protein or peptide, and an antibody, or binding fragment thereof, optionally wherein the antibody is a non-fragmented antibody, a modified antibody, a camelid antibody, a single chain variable fragment, or a Fab fragment. In a specific embodiment, the NACE comprises an antibody or binding fragment thereof, optionally a non-fragmented antibody, a modified antibody, a camelid antibody, a single chain variable fragment, or a Fab fragment, that specifically binds to IgG, e.g., an IgG1, IgG2, IgG3, an IgG4, IgM, IgD, and IgE, but preferably an IgG. In another embodiment, the NACE comprises protein G and/or streptavidin. In another embodiment, the NACE comprises a binding tag, such as a His tag or biotin tag.

In a specific embodiment, the NACE is bound or fused to any other element of the dual-affinity probe and/or on the surface binding domain. For example, the NACE can be bound or fused directly to the surface, the surface beads, the surface binding protein/peptide, and/or the capture element.

Linkers (LI) of Dual-Affinity Probes

Linkers may be included in the dual affinity probes of the present invention. Linkers may include any appropriate amino acid sequence required to control steric hindrance and/or chemical interactions with sensor components (organic or inorganic materials, peptides and proteins, cross-linking reagents, etc.).

The linker sequences of the dual-affinity probes of the present invention may include one or more passive linkers and/or active linkers. In certain embodiments, a dual-affinity probe comprises a passive linker fused to an active linker, e.g., to link the SBM or ISBP, or NACE, to the active linker. As used herein, a passive linker does not specifically bind to a capture element or other polypeptide, and are typically present between two polypeptide sequences to control steric hindrance, e.g., to retain activity of the two linked polypeptides. In particular embodiments, a passive linker may be a single bond or an amino acid sequence that links the SBM, NACE, or ISBP to the CE (or polypeptide comprising the CE). A passive linker may also be present between a CE and a member of a binding pair to which it is fused. The link may be a covalent bond, an ionic bond, a non-covalent bond such as with the use of high-affinity molecules.

As used herein, an active linker may be fused to the SBM or ISBP and specifically binds to a CE or a polypeptide comprising the CE (e.g., a member of a binding pair present in the polypeptide comprising the CE), and may be present to functionally link the SBM or ISBP to the CE. In particular embodiments, an active linker binds to antibodies or antigen-binding fragments thereof (e.g., human antibodies or fragments thereof). In certain embodiments, an active linker is a member of a binding pair, such as streptavidin/biotin. The link may be a covalent bond, an ionic bond, a non-covalent bond such as with the use of high-affinity molecules.

In another embodiment, the linker sequence may include other amino acid sequences, such as passive linkers, a linear tandem repeat polypeptides, a linear non-repeating polypeptides or linkers that allow for additional flexibility or rigidity to the SBM, ISBP or CE.

In a specific embodiment, the high affinity molecule in the linker (i.e., the AL) may be an amino acid sequence comprising protein G from Streptococcus, or an amino acid sequence comprising streptavidin from Streptomyces. In another embodiment, the linkers may include an additional AL to directly and covalently bond to the SBM, ISBP but with a high affinity to IgG or biotin incorporated in the capture element.

In a specific embodiment, the passive linker may include a glycine-serine linker, for example the following amino acid sequence:

[SEQ ID NO: 1]
GGGGSGGGGSGGGGSASGGG

In another specific embodiment, the linker may have the following amino acid sequence:

[SEQ ID NO: 61]
TPTPTTPTPTPTTPTPTPST

In another embodiment, the passive linker of SEQ ID NO: 1 or SEQ ID NO: 61 may be further incorporated or fused with another amino acid sequence on the linker, e.g., an AL, such as a high affinity protein such as streptavidin or protein G. In a specific embodiment, SEQ ID NO: 1 is directly fused to protein G to form the following sequence [SEQ ID NO: 2] as follows:

MTYKLILNGKTLKGETTTEAVDAATAEKVFKQYANDNGVDGEWTYDDATK
TFTVTEKPEVIDASELTPAVTTYKLVINGKTLKGETTTEAVDAATAEKVF
KQYANDNGVDGEWTYDDATKTFTVTEKPEVIDASELTPAVTTYKLVINGK
TLKGETTTKAVDAETAEKAFKQYANDNGVDGVWTYDDATKTFTVTEGGGG
SGGGGSGGGGSASGGG

In this example, the passive linker SEQ ID NO: 1 is on the C terminus of the AL and directly links to the SBM or ISBP, wherein the protein G amino acid sequence binds with high affinity to the capture element, which would be any IgG antibody or appropriate fragment of an IgG antibody.

In another specific embodiment, a passive linker such as SEQ ID NO: 1 may be fused to streptavidin (AL) in the linker. In a specific embodiment, the passive linker SEQ ID NO: 1 is on the C terminus of the AL and directly links to the SBM or ISBP, wherein the streptavidin amino acid sequence binds with high affinity to the biotinylated capture element.

In a specific embodiment, SEQ ID NO: 1 is directly fused to streptavidin to form the following sequence [SEQ ID NO: 21] as follows:

MDPSKDSKAQVSAAEAGITGTWYNQLGSTFIVTAGADGALTGTYESAVGN
AESRYVLTGRYDSAPATDGSGTALGWTVAWKNNYRNAHSATTWSGQYVGG
AEARINTQWLLTSGTTEANAWKSTLVGHDTFTKVKPSAASIDAAKKAGVN
NGNPLDAVQQGGGGSGGGGSGGGGSASGGG

In this example, the passive linker SEQ ID NO: 1 is on the C terminus of the streptavidin AL and directly links to the SBM or ISBP, wherein the streptavidin amino acid sequence binds with high affinity to the capture element (or a polypeptide comprising the CE), which may be a biotinylated protein, including an antibody or antibody fragment.

In a specific embodiment, the ISBP fuse to the linker may be an amino acid sequence or peptide that binds to gold, silicon, cellulose, polystyrene, or silica. In another specific embodiment, the ISBP may be or comprise any one of SEQ ID NO: 3-19 or 25.

In another embodiment, no passive linker is included in the linker sequence. For example, the linker AL, may be specific to just the protein G amino acid sequence or the streptavidin amino acid sequence. In a specific embodiment, the linker (AL) may comprise the following sequence of protein G, [SEQ ID NO: 19] as follows:

MTYKLILNGKTLKGETTTEAVDAATAEKVFKQYANDNGVDGEWTYDDATK
TFTVTEKPEVIDASELTPAVTTYKLVINGKTLKGETTTEAVDAATAEKVF
KQYANDNGVDGEWTYDDATKTFTVTEKPEVIDASELTPAVTTYKLVINGK
TLKGETTTKAVDAETAEKAFKQYANDNGVDGVWTYDDATKTFTVTE

In a specific embodiment, the linker (AL) is SEQ ID NO: 19.

In another embodiment, the linker (AL) may comprise the following sequence of streptavidin [SEQ ID NO: 22] as follows:

MDPSKDSKAQVSAAEAGITGTWYNQLGSTFIVTAGADGALTGTYESAVGN
AESRYVLTGRYDSAPATDGSGTALGWTVAWKNNYRNAHSATTWSGQYVGG
AEARINTQWLLTSGTTEANAWKSTLVGHDTFTKVKPSAASIDAAKKAGVN
NGNPLDAVQQ

In a specific embodiment, the streptavidin sequence may not be directly linked on the N-terminal end of any peptide or protein, and/or may not be directly linked to a methionine residue at the N-terminal end. In a specific embodiment the streptavidin sequence may be as follows:

[SEQ ID NO: 60] as follows:

DPSKDSKAQVSAAEAGITGTWYNQLGSTFIVTAGADGALTGTYESAVGNA
ESRYVLTGRYDSAPATDGSGTALGWTVAWKNNYRNAHSATTWSGQYVGGA
EARINTQWLLTSGTTEANAWKSTLVGHDTFTKVKPSAASIDAAKKAGVNN
GNPLDAVQQ

In another specific embodiment, the linker sequences may be their own fusion protein, or may incorporate other elements of the present invention, such as the SBM or ISBP and/or CE to form a fusion protein. Fusion proteins, including the design, gene synthesis, the cloning, expression, and purification thereof are known in the art, and can be incorporated to form any fusions thereof. For example, the linkers of the present invention may incorporate such sequences with tags or cleavage sites for protein purification, such as His tags or other protein tags known in the art. In another embodiment, the peptides or protein may include protease or cleavage sites. In a specific embodiment, the cleavage site may be a TEV cleavage site. The Examples of the present application provide examples of specific fusion proteins, but is not limited to the invention herein.

In another specific embodiment, the LI linker may just be a single bond, such as a covalent bond. In such an example, the SBM or ISBP and CE are thus directly bonded to each other with no additional amino acid or atom representing the Linker.

Surface Binding Moieties of Dual-Affinity Probes

The dual-affinity probes of the present invention may include a surface binding moiety (SBM) that binds to an organic or inorganic surface of choice. For example, the SBM binds specifically to a biosensor material selected from the group consisting of gold, silica, silver, cellulose, e.g., nitrocellulose, plastic, polystyrene and graphene. In particular embodiments, the SBM is an organic or inorganic surface binding polypeptide (ISBP). As used herein the ISBP may bind to organic or inorganic surfaces. In another example, the ISBP may bind specifically to a biosensor material selected from the group consisting of gold, cellulose, silica and polystyrene.

In a specific embodiment, the SBM or ISBP may include an amino acid sequence and may be selected from the group consisting of a binding peptide, a protein, an antibody with an affinity to the inorganic surface, or an antigen-binding fragment thereof, such as a single chain variable fragment (scFv). In particular embodiments, the inorganic surface binding polypeptide is a peptide. In particular embodiments, the inorganic surface binding polypeptide is an antibody, or an antigen-binding fragment thereof, e.g., an scFv. A variety of surface binding peptides are known in the art, and illustrative surface binding peptides are disclosed herein.

In a specific embodiment, the ISBP comprises a peptide specific to binding gold, cellulose, silicon or polystyrene. In another embodiment, the ISBP comprises a peptide from Table 1 provided herein.

In another embodiment, the ISBP comprises an antibody or a fragment of an antibody. In a specific embodiment, the ISBP is a V_{H} or V_{L} binding motif. In a specific embodiment, the ISBP is a gold V_{H} or V_{L} binding motif. In a specific embodiment, the antibody or a fragment of an antibody may be specific to binding gold. In a specific embodiment, the ISBP may be a gold-binding protein from U.S. Pat. No. 7,807,391, Shiotsuka et al., which is incorporated by reference herein in its entirety.

ISBP-LI-CE (Ia) or (IIa) Dual Affinity Probes

The dual-affinity probe of the present invention may have the following formula (Ia): ISBP-LI-CE (Ia) or formula (IIa): CE-LI-ISBP (IIa).

In a specific embodiment, capture element CE is an organic binding entity specific for the pathogen. The capture element is selected from a single chain variable fragment, a Fab fragment, an antibody, or an antigen; LI is a linker sequence comprising one or more passive linker and/or active linker. In certain embodiments, one or more of the linkers present in LI comprises a single bond, or is selected from one or more of the group consisting of an amino acid linker, an amino acid sequence comprising protein G from Streptococcus, or an amino acid sequence comprising streptavidin from Streptomyces; and the ISBP binds specifically to a biosensor material selected from the group consisting of gold, silica, silver, cellulose, plastic, polystyrene and graphene.

In a specific embodiment, LI is single bond, therein allowing ISBP to bind directly to CE.

In this arrangement, the dual affinity probes may comprise the inorganic surface binding polypeptide and the analyte-specific capture element within the same polypeptide, and may be directly fused to each other or fused to each other via one or more linker, e.g., a passive polypeptide linker. In particular embodiments, the analyte-specific capture element specifically binds to an analyte of interest. In certain embodiments, the analyte-specific capture element is an antibody or an antigen-binding fragment thereof, e.g., such as an scFv.

In a specific embodiment, the dual-affinity probe is a single fusion protein. In another embodiment, the CE and ISBP is independently an antibody, a fragment of an antibody, or a single chain variable fragment from an antibody. In another embodiment, the ISBP is a single chain variable fragment from an antibody. In another embodiment, the single chain variable fragment is a V_{H} binding motif. In a specific embodiment, the V_{H} binding motif is a gold V_{H} binding motif. In another embodiment, the CE is a single chain variable fragment from an antibody. In a specific embodiment, the ISBP and CE are fused as a bispecific antibody fragment.

In a specific combination, the ISBP is a single chain variable fragment that is a V_{H} gold binding motif, and the CE is a single chain variable fragment specific to an antigen.

In another specific combination, the CE and ISBP are each an antibody. In a specific embodiment, the CE and ISBP are fused to form a bispecific immunoglobulin A. In a specific embodiment, the CE and ISBP are fused to form a bispecific antibody fragment. In a specific embodiment, the CE and ISBP are fused to form a bispecific antibody fragment wherein the CE and ISBP or independently a V_{L} fragment, V_{H} fragment and/or a scFv fragment.

In another specific embodiment, the ISBP is specific for gold, silica, silver, cellulose, plastic, polystyrene and graphene. In a specific embodiment, the ISBP is specific for gold.

In another specific embodiment, the CE is specific to an antigen for SARS-CoV-2.

In a specific embodiment, the CE is specific for SARS-CoV-2 Spike (S) antigen or SARS-CoV-2 Nucleocapsid (N) antigen. In another specific embodiment, the CE is an antibody and is an S or N antigen targeting antibody specific for SARS-CoV-2 Spike (S) antigen or SARS-CoV-2 Nucleocapsid (N) antigen.

In another specific combination, the CE and ISBP are each an antibody with a linker in between. In a specific embodiment, the CE and ISBP are fused to form a bispecific immunoglobulin A. In a specific embodiment, the CE and ISBP are fused to form a bispecific antibody fragment. In a specific embodiment, the CE and ISBP are fused to form a bispecific antibody fragment wherein the CE and ISBP or independently a V_{L} fragment, V_{H} fragment and/or a scFv fragment.

In another specific embodiment, the CE is an antigen. In another specific embodiment, the CE is an antigen fused to a linker or SBM/ISBP. In another specific embodiment, the CE antigen is biotinylated and binds to a streptavidin linker. In another specific embodiment, the CE is an antigen that binds to an antibody (or antibodies), the intended analyte for detection. In a specific embodiment, the antigen protein is SARS-CoV-2 Spike and/or SARS-CoV-2 Nucleocapsid proteins. In another specific embodiment, the antigen binds to and detects antibodies. In another embodiment, the antibody or antibodies are a targeting antibody specific for SARS-CoV-2 Spike (S) antigen or SARS-CoV-2 Nucleocapsid (N) antigen, or an antigen-binding fragment thereof.

ISBP-LI-CE (IIIc, IIId, IVc, IVd) Dual Affinity Probes

The dual-affinity probe of the present invention may comprise one or more polypeptide having the formula (IIIc) or (IIId) and one or more polypeptide having the formula (IVc) or (IVd):

ISBP-PL-AL (IIIc)

AL-PL-ISBP (IIId)

ALB-PL-CE (IVc)

CE-PL-ALB (IVd),

wherein LI, AL, and ALB are as defined for formulas (IIIa) and (IVa), and wherein PL may be present or absent from either or both the polypeptide of formula (IIIc) or (IIId) and/or the polypeptide of formula (IVc) or (IVd).

In a specific embodiment, PL comprises an amino acid sequence in between ISBP and CE. In particular embodiments, the AL if the polypeptide of formula (III) and the ALB of the polypeptide of formula (IV) are capable of binding to each or are bound to each other.

In such an arrangement, the inorganic surface binding polypeptide and the analyte-specific capture element may be present in different polypeptides. For example, in certain embodiments, the dual-affinity probes comprise a first polypeptide comprising the inorganic surface binding polypeptide and an active linker (AL), and a second polypeptide comprising the analyte-specific capture element, wherein the AL is capable of binding to the analyte-specific capture element (or a polypeptide comprising the CE). In certain embodiments, the AL directly binds the analyte specific capture element; for example, the AL may be protein A, protein G, or anti-IgG (e.g., goat anti-human IgG), and the analyte specific capture element may be an antibody, or antigen binding fragment thereof. In other embodiments, the analyte specific capture element is fused to a binding element (ALB) that directly binds to the AL; for example, the ALB may be biotin, and the AL may be streptavidin, or vice versa. In certain embodiments, protein G is fused to the N-terminus of the inorganic surface binding polypeptide, e.g., via a passive linker, such as a direct bond or a peptide linker. In certain embodiments, streptavidin is fused to the N-terminus of the inorganic surface binding polypeptide, e.g., via a passive linker, such as a direct bond or a peptide linker. In certain embodiments, protein G is fused to the C-terminus of the inorganic surface binding polypeptide, e.g., via a passive linker, such as a direct bond or a peptide linker. In certain embodiments, streptavidin is fused to the C-terminus of the inorganic surface binding polypeptide, e.g., via a passive linker, such as a direct bond or a peptide linker. Various other binding pairs, in addition to biotin and streptavidin are known in the art, and could alternatively be used.

In a specific embodiment, the ISBP of the dual-affinity probes is selected from the group consisting of a binding peptide, a protein, an antibody with an affinity to the inorganic surface, or an antigen-binding fragment thereof, such as a single chain variable fragment. In a specific embodiment, the ISBP is a binding peptide. In a specific embodiment, the binding peptide is from Table 1 herein.

In another specific embodiment, the ISBP is an antibody, a single chain variable fragment from an antibody or a Fab fragment. In a specific embodiment, the ISBP has a gold binding motif. In another specific embodiment, the ISBP is a V_{H} binding motif. In another specific embodiment, the ISBP is a V_{H} gold binding motif. In another specific embodiment, the ISBP is an antibody specific to binding gold.

In a further specific embodiment, AL is an amino acid sequence comprising protein G from Streptococcus or an amino acid sequence comprising streptavidin from Streptomyces.

In another embodiment, the linker sequences may include other amino acid sequences, such as passive linkers, a linear tandem repeat polypeptides, a linear non-repeating polypeptides or linkers that allow for additional flexibility or rigidity to the ISBP or CE.

In another embodiment, the linker sequences may include an additional passive linker to directly and covalently bond to the ISBP but with a high affinity to IgG or biotin incorporated in the capture element.

In a specific embodiment, the passive linker may include for example the following amino acid sequence:

[SEQ ID NO: 1]
GGGGSGGGGSGGGGSASGGG

The passive linker of SEQ ID NO: 1 may be further incorporated or fused with another amino acid sequence on the linker such as a high affinity protein such as streptavidin or protein G (AL). In a specific embodiment, SEQ ID NO: 1 is directly fused to protein G to form the following sequence [SEQ ID NO: 2] is:

MTYKLILNGKTLKGETTTEAVDAATAEKVFKQYANDNGVDGEWTYDDATK
TFTVTEKPEVIDASELTPAVTTYKLVINGKTLKGETTTEAVDAATAEKVF
KQYANDNGVDGEWTYDDATKTFTVTEKPEVIDASELTPAVTTYKLVINGK
TLKGETTTKAVDAETAEKAFKQYANDNGVDGVWTYDDATKTFTVTEGGGG
SGGGGSGGGGSASGGG

In this example, the passive linker SEQ ID NO: 1 is on the C terminus and directly links to the ISBP, wherein the protein G amino acid sequence binds with high affinity to the capture element, which would be any IgG antibody or appropriate fragment of an IgG antibody.

In another specific embodiment, a passive linker such as SEQ ID NO: 1 may be fused to streptavidin in the linker. In a specific embodiment, the passive linker SEQ ID NO: 1 is on the C terminus and directly links to the ISBP, wherein the streptavidin amino acid sequence binds with high affinity to the biotinylated capture element.

In another embodiment, no passive linker is included in the linker sequences. For example, the linker AL, may be specific to just the protein G amino acid sequence such as SEQ ID NO: 19, variants thereof, or the streptavidin amino acid sequence.

In another specific embodiment, the CE is an antibody. In another specific embodiment, the CE is a fragment of an antibody. In a specific embodiment, and the antibody is conjugated with biotin (ALB), and the AL is an amino acid sequence comprising streptavidin from Streptomyces. In another embodiment, the CE is an antibody and the AL is an amino acid sequence comprising protein G from Streptococcus. In another embodiment, the CE is an antibody and is an S or N antigen targeting antibody specific for SARS-CoV-2 Spike (S) antigen or SARS-CoV-2 Nucleocapsid (N) antigen.
In various embodiments, the dual-affinity probes or immunoprobes are labeled with a detectable label. In particular embodiments of the dual-affinity immunoprobes, the polypeptide comprising the analyte-specific capture element is labeled with a detectable label.

### Analyte Compositions

In a specific embodiment, the dual-affinity probes may be included in a composition, or analyte composition. In a specific embodiment, the analyte composition may comprise any dual-affinity probe described herein. In another specific embodiment, the analyte composition may comprise 1, 2, 3, 4, or 5 different dual-affinity probes as described herein. In a specific embodiment, the analyte composition includes the dual-affinity probe and/or at least one additional binding moiety, such as an antibody or antigen. In another embodiment, the composition may be included on a surface of a system or diagnostic kit. In another specific embodiment, the composition is added to a surface, thereby allowing the dual-affinity probe and/or binding moiety to flow down a surface. In a specific embodiment, the analyte composition is added to a pre-treated sample pad in a lateral flow assay (LFA).

In a specific embodiment, the analyte composition can be free of a dual affinity probe, but includes a binding moiety.

In another embodiment, the analyte composition comprises 1, 2, 3, 4, 5, 6, 7, or 8 different binding moieties.

In a specific embodiment, at least one additional binding moiety comprises a surface particle. In another embodiment, the at least additional binding moiety is not bound to a surface particle. In another embodiment, one binding moiety is a dual-affinity probe and the second binding moiety is not bound to a surface particle. In a specific embodiment, the second binding moiety comprises an antibody or binding fragment thereof, optionally a non-fragmented antibody, a modified antibody, a camelid antibody, nanobody, a single chain variable fragment, or a Fab fragment.

In another embodiment, the additional binding moiety specifically binds to the analyte. In a specific embodiment, the binding moiety is an antibody. In another embodiment, the antibody has a tag such as a biotin tag or a his-tag.

### Analyte Detection Systems and Kits

In one embodiment of the present invention, the dual-affinity probes may be utilized and or included in an analyte detection or diagnostic system, such as a diagnostic kit. In a specific embodiment, this system or kit is used for detecting a specific analyte in a sample.

In certain embodiments, the system or kit comprises an analyte capture element, which binds, directly or indirectly, to the analyte being detected, and a detection element, which binds, directly or indirectly, to the analyte capture element bound to the analyte. In particular embodiments, each of the analyte capture element and the detection element comprises a surface binding moiety, and in particular embodiments, they comprise surface binding moieties that bind to different surfaces. In some embodiments, the analyte capture element comprises an antibody that binds a target analyte, and the surface binding moiety is directly bound or fused to the antibody, optionally via a linker. In some embodiments, the analyte capture element comprises an antibody that binds a target analyte, and the surface binding moiety is indirectly bound to the antibody, e.g., via another binding member, such as an agent that binds to antibody Fc or hinge domains. In some embodiments, the detection element comprises an antibody that binds a target analyte, and the surface binding moiety is directly bound or fused to the antibody, optionally via a linker. In some embodiments, the detection element comprises an antibody that binds a target analyte, and the surface binding moiety is indirectly bound to the antibody, e.g., via another binding member, such as an agent that binds to antibody Fc or hinge domains. The use of agents and systems based on indirect binding of analyte advantageously allows the system to be used with a large variety of different analyte capture elements and detection elements without the need to attach surface binding moieties to them.

The systems or kits may be used for sandwich assays to detect an analyte, wherein the system comprises two antibodies that bind the analyte, at different locations, wherein one antibody is a component of the analyte capture element, and the other antibody is associated with or bound by the detection element. Systems and assays mat also be modified for competition assays.

In particular embodiments, the analyte capture element and/or detection element comprise other binding moieties that bind, directly or indirectly, to the antibody that binds the analyte, to facilitate the formation of a complex comprising the analyte capture element and analyte, or a complex comprising the analyte capture element, the analyte, and the detection element. A variety of binding moieties may be used, including but not limited to, secondary antibodies, and various binding pairs.

In one example, an analyte-binding moiety is tagged with a polypeptide sequence bound by a polypeptide (e.g., an antibody). Such tag sequences are known and available in the art and include, e.g., his tags, flag tags, biotin tags, and myc tags, etc. Such tag sequences may be incorporated into an antibody during its manufacture or synthesis, e.g., as a fusion to the C-terminus of an antibody heavy chain. When an analyte-binding antibody comprises a tag, it may be indirectly bound by a capture element or detection element comprising the polypeptide that binds the tag sequence, e.g., an anti-biotin or anti-his antibody. In particular embodiments, the detection element comprises the anti-tag antibody, wherein the anti-tag antibody comprises a surface binding moiety, e.g., fused to the C-terminus of its Fc domain. As another example, one element may comprise a member of a binding pair, such as streptavidin, avidin, or neutravidin, which bind biotin tag.

As another example, one element may comprise a secondary antibody that binds to only one of the analyte-binding antibodies, and the other element may comprise a secondary antibody that binds only to the other analyte-binding antibody. This may be accomplished a variety of manners, e.g., one analyte-binding antibody may comprise a human Fc bound by a particular secondary antibody, while the other analyte-binding antibody may comprise an Fc of a different species bound by a different secondary antibody. In other embodiments, one antibody may be IgG and the other IgM or IgA, which are bound by different proteins, with IgG being bound by protein G, IgM by protein L and IgA by jaculin.

Compositions, systems, kits and methods disclosed herein may be used in lateral flow assays. For example, in certain assays, a detection element is bound to a lateral flow surface, such as nitrocellulose, at a first location. An analyte capture element (or portion thereof) is present at a different second location of the lateral flow surface, e.g., in a lyophilized form. When a test sample is applied to the second location (in liquid), it mixes with the analyte capture element, which binds analyte present in the sample. The liquid comprising the analyte capture element flows across the lateral flow surface to the first location, where the analyte capture element bound to analyte is bound by the detection element. In particular embodiments, the analyte capture element is bound to, or associated with, a nanoparticle or bead, e.g., a gold bead, that facilitates its detection at the second location. Thus, in particular embodiments, the analyte capture element comprises a nanoparticle, e.g., gold, surface binding moiety, and the analyte capture element comprises a surface binding moiety that binds to the lateral flow surface, e.g., a cellulose or nitrocellulose surface binding moiety.

The assays and systems disclosed herein may adopt a variety of different configurations, including but not limited to, those specifically described herein or diagrammed in the accompanying Examples and figures. For example, in each case, any of the antibodies may instead be a molecularly imprinted polymer (MIP) or aptamer or engineered protein.

In certain embodiments, the systems, reagents, and methods disclosed herein may be adapted to detect a small analyte, e.g., an analyte to which two different antibodies cannot bind at the same time. Examples of such small analytes include, but are not limited to, peptides, toxins, drugs, metal ions, hormones. In certain embodiments, the small analyte has an Mr<1000. In certain embodiments, this is accomplished by forming an antibody sandwich-type complex with the small analyte, by using two antibodies, wherein a first antibody binds to the small analyte, and the second antibody binds to the first antibody only when the first antibody is bound to the small analyte. In one embodiment, binding of the first antibody to the small analyte causes a conformational change in the first antibody, thus unmasking the epitope bound by the second antibody. Antibodies that bind small analytes may be generated by immunizing an animal with the small analyte conjugated to a hapten. Second antibodies that bind to the first antibody only when it is bound to the small analyte may be generated or identified by screening antibody libraries.

In a specific embodiment, the system may comprise a) an analyte capture element, comprising an antibody associated with a particle, such as a nanoparticle, or bead, wherein the analyte capture element binds to the analyte; b) a sandwich element, comprising an antibody that binds to the analyte; and c) a lateral flow capture element, wherein the lateral flow capture element is a fusion polypeptide comprising a first surface binding moiety fused to a binding member. In another embodiment, the analyte capture element is directly or indirectly physisorbed to the nanoparticle or bead. In one embodiment, the analyte capture element is directly or indirectly bound to the nanoparticle or bead, e.g., via a surface binding moiety. In another embodiment, the analyte capture element is directly or indirectly physisorbed to the nanoparticle or bead. In another embodiment, the analyte capture element is directly or indirectly bound to the nanoparticle or bead. In a specific embodiment, the analyte capture element comprises a second surface binding moiety that directly binds to the nanoparticle or bead. In another embodiment, the analyte capture element is bound by a secondary binding moiety that comprises a second surface binding moiety that is directly bound to the nanoparticle or bead. In another specific embodiment, the surface binding moiety binds to cellulose or nitrocellulose. In another embodiment, the lateral flow capture element includes a surface binding moiety comprising a polypeptide having a sequence set forth in Table 1. In another specific embodiment, the surface binding moiety is fused to an additional polypeptide sequence thereby having a sequence set forth in Table 1a.

In a specific embodiment, the system may include: a) a first antibody bound to a nanoparticle surface via a first surface binding moiety fused to an Fc region of the first antibody, wherein the first antibody binds to the analyte; b) a second antibody comprising a tag polypeptide sequence, optionally a Biotin tag polypeptide sequence or a His tag polypeptide sequence, wherein the second antibody binds to the analyte; c) a third antibody bound to a second surface, optionally a cellulose or nitrocellulose surface, via a second surface binding moiety fused to an Fc region of the third antibody, wherein the third antibody binds to the tag polypeptide sequence present in the second antibody, wherein the first antibody and the second antibody bind to different epitopes of the analyte and do not compete with each other for binding to the analyte. In another embodiment, the system may include: a) a first antibody that binds to the analyte; b) a second antibody that binds to the analyte, wherein the first antibody and the second antibody bind to different epitopes of the analyte and do not compete with each other for binding to the analyte; c) a third antibody bound to a nanoparticle surface via a first surface binding moiety fused to an Fc region of the third antibody, wherein the third antibody binds to the first antibody; and d) a fourth antibody bound to a first region of a surface, optionally a cellulose or nitrocellulose surface, via a second surface binding moiety fused to an Fc region of the fourth antibody, wherein the fourth antibody binds to the second antibody, but does not bind to the first antibody. In another embodiment, the system may further include e) a fifth antibody bound to a second region of the surface via a third surface binding polypeptide fused to an Fc region of the fifth antibody, wherein the fifth antibody binds to the first antibody, but does not bind to the second antibody

In another specific embodiment, the detection systems may be a detection or diagnostic kit. In a specific embodiment, the kits may be kits for detecting an analyte, and may be for a point of care service, a kit for detecting an analyte sample in a hospital/clinic setting, or kit for detecting an analyte in a sample that can be used at home. In a specific embodiment, the kit may be an assay, such as an immune assay. In another embodiment, the kit may be a dipstick assay. In another embodiment, the kit may be a lateral flow assay. In a specific embodiment, the kit is a diagnostic kit, meaning the kit can be used in a setting described above for detecting an analyte in a sample.

In a specific embodiment, the kit or diagnostic kit includes: a) a surface, b) a surface binding moiety (SBM) bound to at least a portion of the surface, c) a reagent for collecting the sample; and d) an analyte composition comprising a set of at least one dual affinity probe (DAP).

In another example, the kit includes: a) a surface; b) a surface binding moiety (SBM) bound to at least a portion of the surface, wherein the SBM comprises an surface binding peptide (SBP), and/or optionally a non-analyte capture element (NACE); c) a reagent for collecting the sample; and d) an analyte composition comprising a set of at least one dual affinity probe (DAP), wherein at least one DAP is any one of the dual-affinity probes described herein. In a specific embodiment, the dual affinity particle includes a gold surface particle.

In a specific embodiment, the surface of the kit can be one or more selected from the group consisting of PDMS, ZnO, gold, quartz, silica, silicon, silver, cellulose, nitrocellulose, plastic, polystyrene and graphene. In a specific embodiment of the kits herein, the SBP comprises a nitrocellulose binding protein, a silicon binding protein, a cellulose binding motif, a polystyrene binding motif, and/or a silica binding motif. In another embodiment, the SBP comprises a sequence selected from Table 1. In another specific embodiment, the SBP comprises a silicon binding protein of EMT-020 to EMT-025, the cellulose binding motif is cellulose binding motif 1 or cellulose binding motif 2, and the polystyrene binding motif is polystyrene binding motif 1 or polystyrene binding 2, and/or the silica binding motif 1.

In another embodiment of the kits, the SBM comprises one or more NACE selected from the group consisting of a linker, streptavidin, protein G, biotin, biotinylated protein, an antigen protein or peptide, and an antibody, wherein the antibody is an antibody or binding fragment thereof, optionally a non-fragmented antibody, a modified antibody, a camelid antibody, a single chain variable fragment, or a Fab fragment.

In another specific embodiment, the SBM comprises an SBP fused or bound to an NACE, wherein the fusion protein is selected from one or more of the group consisting of Table 1a.

In another embodiment, the analyte composition is any analyte composition described herein. In another embodiment, the analyte composition is provided in lyophilized beads. In another embodiment, the kits herein include at least 2, 3, 4, or 5 analyte compositions.

In a specific embodiment, the kit additionally comprises at least a second analyte composition comprising a second analyte binding moiety (SABM), wherein the SABM can comprise one or more selected from a surface particle, an SBP, a NACE, and a CE. In one embodiment, the second analyte composition can comprise any dual-affinity probe described herein. In a specific embodiment, the SABM comprises a NACE, wherein the NACE is one or more selected from the group consisting of a linker, streptavidin, protein G, biotin, an antigen protein or peptide, and an antibody, wherein the antibody is an antibody or binding fragment thereof, optionally a non-fragmented antibody, a modified antibody, a camelid antibody, a single chain variable fragment, or a Fab fragment.

In a specific embodiment, the second analyte composition comprises a surface particle as a lyophilized analyte bead. In another embodiment, the second analyte composition comprises a surface particle that is bound to an SBP that is fused or bound to a non-analyte capture element (NACE), or a surface particle fused or bound to a NACE. In a specific embodiment, the NACE is one or more selected from the group consisting of a linker (LI), streptavidin, protein G, biotin, an antigen protein or peptide, and an antibody, wherein the antibody is an antibody or binding fragment thereof, optionally a non-fragmented antibody, a modified antibody, a camelid antibody, a single chain variable fragment, or a Fab fragment.

In a specific kit, the analyte composition comprises a dual-affinity probe as described herein, and the second analyte composition comprises a SABM. In a specific embodiment of the kit both compositions are incorporated on the surface of the kit. In another embodiment, the compositions are placed on the same area of the kit or placed next or adjacent to each other on the kit. In a specific embodiment, there is a spacer, i.e., just the surface separating the two compositions. In a specific embodiment, the compositions are placed on the surface of a pre-treated sample pad and comprises a surface selected from one or more selected from the group consisting of PDMS, ZnO, gold, quartz, silica, silicon, silver, cellulose, nitrocellulose, plastic, polystyrene and graphene.

In another embodiment, the surface of the kit may include a control surface binding moiety. In a specific embodiment, the control surface binding moiety allows for a positive control indication or negative control indication for a user of the kit. In a specific embodiment, the analyte composition or the second analyte composition comprises a control binding moiety that binds to the control surface binding moiety. The control surface binding moiety may be tested near or adjacent to the line or area comprising the SBP, thereby providing a control near the binding of the DAP to the surface binding moiety bound to the surface. In a specific embodiment, the control binding moiety in the analyte composition or the second analyte composition comprises a surface particle. In another embodiment, the control binding moiety in the analyte composition or the second analyte composition comprises an SBP or NACE bound to the surface particle. In another embodiment, the control binding moiety in the analyte composition or the second analyte composition comprises a tag, such as biotin, his tag, or protein G. In another embodiment, the control binding moiety comprises an antibody, wherein the antibody is an antibody or binding fragment thereof, optionally a non-fragmented antibody, a modified antibody, a camelid antibody, a single chain variable fragment, or a Fab fragment. In another embodiment, the control binding moiety in the analyte composition or second analyte composition comprises a control antibody or a surface particle bound or fused to a control binding antibody, such as an IgY antibody, wherein the antibody is an antibody or binding fragment thereof, optionally a non-fragmented antibody, a modified antibody, a camelid antibody, a single chain variable fragment, or a Fab fragment. In another embodiment, the control surface binding moiety binds to the control binding moiety to indicate that the assay was administered to run properly, such as in a positive control. For example, in one embodiment, the DAP may comprise a biotin-tagged moiety that once run on the assay, binds to a streptavidin bound, fused or anchored to the surface of the assay. Accordingly, in one embodiment, the control surface binding moiety and the control binding moiety are designed to bind to each other such as a 1) biotin-streptavidin, 2) protein G-IgG antibody, and 3) anti-IgG antibody-IgG antibody binding relationship.

In another embodiment of the kits, the kits may be in the form of a lateral flow assay. In a specific embodiment, the LFA includes: a) a surface, b) a surface binding moiety (SBM) bound to at least a portion of the surface, c) a reagent for collecting the sample; and d) an analyte composition comprising a set of at least one dual affinity probe (DAP). In another embodiment, the LFAs may be used in a form such as exemplified in examples 20-27 herein.

An exemplary assay that utilizes (nitro)cellulose-binding streptavidin fusion proteins (e.g., and dual-affinity probes as described herein for detecting or quantifying the analyte level in a test sample, such as SARS-CoV-2 Nucleocapsid or spike protein. The assay is manufactured into a diagnostic kit using the dual-affinity probes according to the methods described above. The kit may have a pre-treated sample pad section for collecting and reacting the biological sample from a subject, such as human; a surface, such as a nitrocellulose membrane portion where the detection complex is formed on a test line; and an absorbent pad at the end of the strip to collect the flow-through. In a specific embodiment, the pre-treated sample pad comprises an analyte composition comprising a DAP for binding to the analyte. The sample pad is treated with the analyte composition immediately prior to using the kit, or alternatively, the beads are added during the manufacturing of the kit.

The test sample having the analyte of interest may be collected from a subject by using a variety of collection methods (e.g., collecting spat sample in a tube, taking a nasal swab using the assay strip, etc.) and the collected test sample is introduced to a reagent solution. The reagent solution comprising the test sample is then directly applied onto the pre-treated sample pad. The sample, including the analyte, lateral flows on the surface to and then contacts the analyte composition comprising the DAP, allowing for example the DAP beads to dissolve in the sample reagent and bind to the analyte. DAP bound to the analyte then flows laterally on the surface of the surface, such as a nitrocellulose membrane, where the DAP bound to the analyte is captured on the test line by forming complexes (i.e., detection conjugates) with immobilized surface binding moiety bound to the surface. The presence of the detection conjugate captured at the test line is observed with the naked eye to determine the presence and/or quantity of the analyte in the test sample.

In a specific embodiment, the LFA can utilize a kit or system as described herein. In another embodiment, the LFA can utilize a DAP, additional binding moiety, or analyte composition, or more than open analyte composition described herein. In a specific embodiment, the increase in signal provides a method for detection or quantification of the analyte in a sample.

In a specific embodiment, the LFA kits may include one or two analyte compositions. In another embodiment, the compositions may use gold surface particles, as in Examples 20-27 described herein. In a specific embodiment, the LFA kits may utilize an antigen, or recombinant antigen in one or more analyte compositions. In a specific embodiment, the use of the antigen is for the DAP to compete with the analyte in a sample. In a specific embodiment, the analyte in the sample outcompetes the antigen in the analyte composition for binding to the DAP. In such a system, the binding of the analyte, thereby reducing the binding of the antigen allows for a competition assay where the signal is greater or reduced based on the amount of analyte in the sample. In a specific embodiment, the analyte binds to a binding moiety without a surface particle, thereby reducing the signal in the assay. In other words, the reduction in signal provides a method for detection or quantification of the analyte in a sample.

### Methods for Detecting Analyte

The disclosure also provides a method of determining the presence of and/or quantifying an analyte in a test sample, comprising:
contacting a test sample with a dual-affinity probe, wherein the dual-affinity probe comprises a SBM, e.g., an inorganic surface binding peptide (ISBP), and an analyte-specific capture element, under conditions and for a time sufficient for analyte present in the test sample to bind to the analyte-specific capture element, thereby forming complexes comprising the analyte bound to the dual-affinity probe;
determining the presence of and/or quantity of the complexes and/or analyte present in complexes;
wherein the presence of the complexes and/or analyte indicates the presence of the analyte in the test sample, and the quantity of the complexes and/or analyte indicates the quantity of analyte present in the test sample,
thereby determining the presence of and/or quantifying the analyte in the test sample.

In some embodiments, the test sample is a biological sample, such as a biological sample obtained from a subject, such as, e.g., serum, plasma, whole blood, saliva, mucus, nasal fluid, nasopharyngeal secretions, middle ear fluid, cerebrospinal fluid, sweat, urine or a combination thereof. In some embodiments, the subject is a mammal, e.g., a human. In some embodiments, the biological sample comprises pathogens, antibodies, cells, and/or other biological molecules. The method may be used to test a variety of different types of samples, including, e.g., environmental samples (including samples collected in the built environment), water, or food or beverage samples, etc.

Methods of the disclosure may be used to assay for a variety of different analytes in a test sample. Examples of analytes include, but are not limited to, infectious agents, pathogens, antibodies that bind pathogens, specific cells, proteins, or carbohydrates. In certain embodiments, the analyte is an infectious agent or pathogen, and in certain embodiments, the infectious agent or the pathogen is a virus, a bacterium, a fungus, a protozoan, a worm, or a prion. In particular embodiments, the virus is an influenza virus or a coronavirus, e.g., SARS-CoV-2 virus. In other embodiments, the analyte is an antibody that specifically binds to one or more infectious agents or pathogens

The methods may also use a capture element that is an amino acid sequence that is not an antibody or antibody fragment, but any amino acid sequence, peptide, protein or specific antigen that binds to an antibody from the pathogen. For example, the capture element may be used to test a biological sample obtained from a subject to determine if the subject has antibodies for a specific pathogen, and more specifically a specific antigen or epitope that identifies the pathogen. In a specific embodiment, the capture element comprises an antigen or epitope thereof. For example, a biotinylated SARS-CoV-2 Spike protein antigen may be conjugated to the streptavidin fusion protein for the detection of Spike protein specific antibodies in test samples.

The capture element may be specific to any analyte or pathogen of interest, for example, the capture element may be specific to an antigen, protein, peptide, antibody or antibodies, or other organic element that identifies that a subject may be positive for or infected with a specific pathogen. In certain embodiments, the capture element is specific for an antibody that specifically binds an analyte or pathogen of interest. In a specific embodiment the capture element comprises an antigen for SARS-CoV-2. In another specific embodiment, the capture element comprises SARS-CoV-2 Spike (S) antigen or SARS-CoV-2 Nucleocapsid (N) antigen or a variant thereof.

In various embodiments, the analyte-specific capture element specifically binds to an analyte of interest, in order to determine whether it is present in the test sample and/or the amount or concentration present in the test sample. In particular embodiments, the analyte-specific capture element comprises antibodies, or antigen-binding fragments thereof, specific for a pathogen or an antigen thereof, e.g., a SARS-CoV-2 Spike (S) antigen or a SARS-CoV-2 Nucleocapsid (N) antigen.

In particular embodiments, the inorganic surface binding peptide comprises one or more gold-, silver-, silica-, plastic-, cellulose- or graphene-binding peptides, including but not limited to any of the peptides of Table 1 herein.

In certain embodiments, the dual-affinity immunoprobe is bound to an inorganic surface via the inorganic surface binding peptide, and the test sample when the test sample is contacted with the dual-affinity immunoprobe. One example is a lateral flow assay. However, in other embodiments, the dual-affinity immunoprobe is not bound to the inorganic surface when the test sample is contacted with the dual-affinity immunoprobe. For example, the dual-affinity immunoprobe and the test sample may be contacted in a solution and form complexes, and the solution is then contacted with the inorganic surface, such that the dual-affinity immunoprobes to bind to the inorganic surface. In particular embodiments, the inorganic surface is a biosensor material selected from the group consisting of gold, silica, silver, cellulose, plastic, and graphene. Bound complexes or analytes may be detected and/or quantified via various means, for example using quartz crystal microbalance, surface plasmon resonance (SPR), or lateral flow.

In various embodiments, the methods may employ the use of one or more positive or negative control, e.g., a positive control test sample, a negative control test sample, and/or a negative control dual-affinity immunoprobe, an analyte-specific capture element that does not bind the analyte of interest.

In particular embodiments, the analyte is determined to be present in the test sample if it is detected in the test sample, or if a certain level or amount is determined to be present in the test sample. For example, the level or amount that indicates the presence of the analyte in the test sample may be a predetermined amount based on prior experience, or it may be an amount greater than the amount determined using a negative control, e.g., an amount at least 10%, at least 20%, at least 50%, at least two-fold, or an amount at least three-fold greater than the amount determined for a negative control.

In a specific embodiment, this detection of an analyte, i.e., confirmation of the subject being positive with the analyte, may determined by a binding curve, such as by SPR or QCM-D. In other words, the analyte is determined to be present such as obtaining a certain RU or other response or detection curve. In another embodiment, the analyte is determined to be present by a contrast from the negative control in color. Such contrast can be determined by visual determination of individual as instructed in the directions of the assay. Such determination can be performed in a point of care, hospital, or other healthcare facility. In another embodiment, the analyte is determined to be present by a contrast from the negative control in color by a device, such as a multiwell plate color reader.

The accompanying Examples are illustrative regarding certain specific embodiments of the compositions and methods disclosed herein.

Oriented loading of antibodies onto inorganic binding entity was achieved in one embodiment by adsorbing it to protein A and G, which contain binding domains for the Fc (Fragment crystallizable) region of antibodies.

In other embodiments, directed immobilization of recognition biomolecules (e.g., capture elements) is accomplished using the streptavidin-biotin system, which shows one of the strongest non-covalent interactions in nature.

In another embodiment, fusion proteins containing the inorganic binding peptide were linked to a single chain variable fragment (scFv) or a Fab fragment or a full-length antibody for the pathogen of interest. These methods may be employed in engineering dual-affinity immunoprobes of the invention. Other methods of reversibly and irreversibly binding antibodies and known in the art and are set out in detail in (MAKARAVICIUTE; RAMANAVICIENE, 2013) and (LIEBANA; DRAGO, 2016).

Inorganic surface binding peptides may include those that specifically bind to gold, silica and graphene, as well as cellulose, silver, and carbon based synthetic polymers (plastics).

Sensor types may include planar gold, silver, and silica; gold and silver nanoparticles (nanoclusters, nanorods, etc.); graphene sheets and tubes; cellulose sheets and strips; etched plastic sheets and slides, for example. Biosensor material includes gold, silver, silica, graphene, cellulose, and carbon based synthetic polymers, for example.

Pathogens may include Coronavirus spp. Such as SARS and MERS; Influenza spp.; Respiratory Syncytial Virus spp.; Adenovirus spp.; Parainfluenza spp.; Filoviridae such as Ebola and Marburg; Hantavirus spp.; Arenaviridae such as Lassa; Bunyaviridae such as Rift Valley and Crimean-Congo; and Paramyxoviridae such as Hendra and Nipah; for example. Pathogens include, in some embodiments, prions. Pathogens include, in some embodiments, Gram negative and Gram positive bacteria.

Antibody types may include but are not limited to humanized, monoclonal, polyclonal, and synthetic antibodies.

Detection methods using the dual-affinity immunoprobes of the invention include but are not limited to lateral flow, in multiwell plate color readers; dipstick color change, SPR and Quartz crystal microbalance with dissipation monitoring (QCM-D).

In a specific embodiment, the detection methods may use any system or kit described herein. In a specific embodiment, the methods include the method of determining the presence of and/or quantifying an analyte in a sample from a subject, by testing the sample with any one of the diagnostic kits or systems described herein comprising: a) contacting a test sample with the reagent, b) applying the test sample with the reagent to the surface thereby allowing the test sample to flow laterally on the surface thereby contacting the DAP composition on the surface c) allowing for the analyte present in the test sample to bind directly to the CE, thereby forming complexes comprising the analyte bound to the DAP; d) allowing for the analyte complexed to the DAP to further flow laterally on the surface, thereby complexing to the surface binding moiety (SBM); and e) wherein the presence of the DAP complexed to the SBM determines the presence and/or quantity of the analyte in the test sample. In a specific embodiment, the surface comprising the DAP composition is a pre-treated sample pad. In another embodiment, the test sample in the reagent is applied to the pre-treated sample pad. In another specific embodiment, the pre-treated sample pad has a different surface membrane than the surface comprising the surface binding moiety (SBM). In another specific example, the pre-treated sample pad comprises a second analyte composition comprising a SABM, and allowing the test sample to flow laterally on the surface and bind to the SABM in the second analyte composition.

In another embodiment, the methods may include determining the presence of and/or quantifying an analyte in a sample from a subject, by testing the sample with any one of the diagnostic kits of described herein comprising: a) contacting a test sample with the reagent, b) applying the test sample with the reagent to the surface thereby allowing the test sample to flow laterally on the surface thereby contacting the DAP composition on the surface; wherein the DAP composition comprises: i) a surface particle fused or bound to an antigen; and ii) an anti-analyte antibody wherein the anti-analyte antibody binds to the antigen, b) allowing for the analyte present in the test sample and the surface particle fused or bound to an antigen to competitively bind to the anti-analyte antibody, c) thereby allowing two complexes wherein the anti-analyte antibody is complexed to the analyte in the test sample and/or the surface particle fused or bound to an antigen the forming complexes comprising the analyte bound to the DAP, d) wherein the more analyte in the sample means that more of the anti-analyte antibody is complexed to the analyte in the test sample and less complexed to the gold particle fused or bound to an antigen, and e) wherein the sample further flows laterally on the surface, wherein the SBM binds to a tag on the anti-analyte antibody, thereby allowing the anti-analyte antibody to specifically complex to the surface binding moiety (SBM) on the surface. In a specific embodiment, the tag is a his-tag or biotin tag. In a specific embodiment, the surface particle is a gold particle and is fused or bound to an antigen and the gold particle and anti-analyte antibody are in separate compositions and the two compositions are applied adjacently on the surface. In another embodiment, the second composition comprising the anti-analyte antibody further comprises a control antibody fused or bound to a gold particle, wherein after the sample further flows laterally on the surface, the control antibody fused or bound to a gold particle binds to an Anti-control antibody on the surface, thereby providing a positive control on the diagnostic kit.

The methods may also include methods of determining the presence of and/or quantifying an analyte in a sample from a subject, by testing the sample with any one of the diagnostic kits described herein by: a) contacting a test sample with the reagent, b) applying the test sample with the reagent to the surface thereby allowing the test sample to flow laterally on the surface thereby contacting the DAP composition on the surface; wherein the DAP composition comprises: i) a gold particle fused or bound to an antigen and; ii) an anti-analyte antibody wherein the anti-analyte antibody binds to the antigen, c) allowing for the analyte present in the test sample and the gold particle fused or bound to an antigen to competitively bind to the anti-analyte antibody, d) thereby allowing two complexes wherein the anti-analyte antibody is complexed to the analyte in the test sample and/or the gold particle fused or bound to an antigen the forming complexes comprising the analyte bound to the DAP; e) allowing for the complex to further flow laterally on the surface, contacting a second composition, wherein the second composition comprises an anti-IgG antibody, thereby allowing the anti-IgG antibody to bind to the anti-analyte antibody, and f) wherein the sample further flows laterally on the surface, and wherein the SBM binds to a tag on the anti-analyte antibody or the anti-IgG antibody, thereby allowing the anti-analyte antibody to specifically complex to the surface binding moiety (SBM). In a specific embodiment, the tag is a his-tag or biotin tag. In another embodiment, the tag is on the anti-IgG antibody. In another embodiment, the second composition comprising the anti-IgG antibody further comprises an IgY antibody fused or bound to a gold particle, wherein after the sample further flows laterally on the surface, the IgY antibody fused or bound to a gold particle binds to an Anti-IgY antibody, thereby providing a positive control on the diagnostic kit.

In another embodiment, the methods may include a test sample that is a biological sample obtained from a subject. In a specific embodiment, the subject is a mammal, optionally a human. In another embodiment, the biological sample comprises serum, plasma, whole blood, saliva, mucus, or a combination thereof. In another embodiment, the analyte is a pathogen. In a specific embodiment, the pathogen is a virus, a bacterium, a fungus, a protozoan, a worm, or a prion. In a specific embodiment, the virus is a SARS-CoV-2 virus. In another specific embodiment, the analyte is a SARS-CoV-2 Spike (S) antigen or a SARS-CoV-2 Nucleocapsid (N) antigen or antibodies specific to binding to SARS-CoV-2 Spike (S) antigen or SARS-CoV-2 Nucleocapsid (N) antigen.

The present invention will be more readily understood by referring to the following examples which are given to illustrate the invention rather than to limit its scope

Acronyms or short forms used in the Examples
H=hours
Min=minutes
s=seconds
PBS=Phosphate Buffered Saline
E. coli=Escherichia coli
SARS-CoV-2=severe acute respiratory distress coronavirus 2
BSA=Bovine Serum Albumin
ddH2O=double distilled water

### EXAMPLE 1

### General Methods

Identification and Synthesis of Synthetic peptides: Six gold-binding and six silica-binding peptides from the literature were contract synthesized with a purity of >90% using FMOC (Fluorenylmethyloxycarbonyl chloride) synthesis (Pierce ThermoFisher).

Design of fusion proteins: The general structure of the embodiments of the invention is inorganic surface binding peptide plus linker plus protein G′, a known version of protein G where the albumin binding site has been removed (a version of Uniprot Q54181 protein.). The Amino acid sequence of this linker plus protein G′ [SEQ ID NO: 2] is:

MTYKLILNGKTLKGETTTEAVDAATAEKVFKQYANDNGVDGEWTYDDATK
TFTVTEKPEVIDASELTPAVTTYKLVINGKTLKGETTTEAVDAATAEKVF
KQYANDNGVDGEWTYDDATKTFTVTEKPEVIDASELTPAVTTYKLVINGK
TLKGETTTKAVDAETAEKAFKQYANDNGVDGVWTYDDATKTFTVTEGGGG
SGGGGSGGGGSASGGG

Antibodies and antigens: Monoclonal antibodies against the SARS-CoV-2 Spike protein (A02038), SARS-CoV-2 Nucleocapsid protein (A02039), and recombinant Spike (Z03501) and Nucleocapsid (Z03488) protein antigens, were purchased from Genscript (Piscataway, NJ).

Quartz Crystal Microbalance with Dissipation Monitoring (QCM-D) for Comparative Peptide Binding Analysis:

The Quartz Crystal Microbalance with dissipation monitoring (QCM-D) is an instrument that measures mass and viscosity in at or near surfaces and thin films. QCM-D can detect extremely small chemical, mechanical, and electrical changes taking place on a sensor surface, and convert them into electrical signals which can be interpreted (TONDA-TURO; CARMAGNOLA; CIARDELLI, 2018).

All QCM-D analyses were performed at 23° C. on the 4-channel Qsense™ Analyzer instrument (Biolin Scientific, Gothenburg, Sweden). The gold and silica Qsense™ sensor chips were rinsed in 70% ethanol, rinsed with deionized water, dried with compressed nitrogen, and then exposed to UV/ozone for 10 min to remove remaining organic residues. Samples were diluted to 100 μg/mL in 10 mM of PBS. The gold or silica sensor chips were loaded into the instrument and equilibrated for 15 min. Ten mM PBS was then flowed at 50 μL/min until an equilibrium for frequency and dissipation D Δfₙ was attained.

The respective gold-binding and silica-binding peptides were flowed over their respective gold and silica sensors for 1 h, followed by a 10 mM PBS wash step for 30 min. The raw data was analyzed using Qsense™ Dfind™ analysis software using a Kelvin-Voigt viscoelastic model.

Surface Plasmon Resonance (SPR) Analysis:

Surface Plasmon Resonance occurs when polarized light hits a metal film at the interface of media with different refractive indices. SPR techniques excite and detect collective oscillations of free electrons, by which light is focused onto a metal film through a glass prism and the reflection is detected. At a certain incident angle (or resonance angle), the electrons (aka plasmons) are set to resonate, resulting in absorption of light at that angle. This creates a dark line in the reflected beam.

The resonance angle can be determined by observing the SPR reflection intensity. A shift in the reflectivity curve represents a molecular binding event taking place on or near the metal film, or a conformational change in the molecules bound to the film. The shift vs. time provides information about molecular binding events and binding kinetics.

All SPR experiments were performed on an 8-channel Biacore™ 8K instrument (Cytiva Lifesciences (was GE Healthcare Lifesciences)), Marlborough, MA, USA) at 25° C. using the 2×HBS-EP+ running buffer and chips from the Biocore™ SIA AU kit (Cytiva Lifesciences).

### EXAMPLE 2

Generation and purification of gold-binding and silica-binding fusion proteins in Escherichia coli: The protein sequences for the fusion proteins, containing well described gold-binding (BROWN, 1997) and silica-binding (ETESHOLA; BRILLSON; LEE, 2005) peptides fused to a linker and the protein G′ protein from Streptococcus, were converted to cDNA using codon usage specific for E. coli. An N-terminal 6× histidine tag to the proteins were added for purification purposes. The cDNA inserts representing the fusion proteins were cloned in frame into the E. coli pET-30a (+) expression vector. Standard molecular cloning techniques were applied to identify the correct clones for protein expression. (SAMBROOK; FRITSCH; MANIATIS, 1989) The recombinant proteins were isolated from the supernatant of 1 L expression cultures following a four-step purification protocol including Ni column, TEV protease digestion, Ni column and finally Q Sepharose column (all reagents from Genscript, Piscataway, NJ). The purity of the proteins was estimated by densitometric analysis of a Coomassie Blue-stained SDS-PAGE gel, and endotoxin levels were assessed using the LAL Endotoxin Assay Kit (Xiamen Bioendo Technology Co., Ltd., Xiamen, Fujian, China).

TABLE 1
SBP Sequences

| | | | | Molecular | | |
|---|---|---|---|---|---|---|
| | | | Length | weight | | SEQ ID |
| ID | Target | Peptide Sequence | (aa) | (Dalton) | Reference | NO: |
| EMT014 | Gold | MHGKTQATSGTIQSMHG | 42 | 4303.752 | (KULP; | SEQ ID |
| | | KTQATSGTIQSMHGKTQ | | | SARIKAYA; | NO: 3 |
| | | ATSGTIQS | | | EVANS, | |
| | | | | | 2004) | |
| EMT015 | Gold | MHGKTQATSGTIQSMHG | 98 | 10018.0684 | (BROWN, | SEQ ID |
| | | KTQATSGTIQSMHGKTQ | | | 1997) | NO: 4 |
| | | ATSGTIQSMHGKTQATS | | | | |
| | | GTIQSMHGKTQATSGTIQ | | | | |
| | | SMHGKTQATSGTIQSMH | | | | |
| | | GKTQATSGTIQS | | | | |
| EMT016 | Gold | WAGAKRLVLRRE | 12 | 1454.7371 | (HNILOVA; | SEQ ID |
| | | | | | OREN; | NO: 5 |
| | | | | | SEKER; | |
| | | | | | WILSON | |
| | | | | | et al., | |
| | | | | | 2008) | |
| EMT017 | Gold | HFSSWETQQG | 10 | 1206.2336 | (TANAKA; | SEQ ID |
| | | | | | | NO: 6 |
| EMT018 | Gold | WYEKWQKANW | 10 | 1438.6042 | HIKIBA* | SEQ ID |
| | | | | | YAMASHITA; | NO: 7 |
| | | | | | MUTO | |
| | | | | | et al., | |
| | | | | | 2017) | |
| EMT019 | Gold | VSGSSPDS | 8 | 734.716 | (HUANG; | SEQ ID |
| | | | | | CHIANG; | |
| | | | | | LEE; | |
| | | | | | GAO et | |
| | | | | | al., | NO: 8 |
| | | | | | 2005) | |
| EMT020 | Silicon | SSKKSGSYSGSKGSRRIL | 36 | 3541.8909 | (KROGER; | SEQ ID |
| | | GGGGMHGKTQATSGTIQ | | | DEUTZMANN; | NO: 9 |
| | | S | | | SUMPER, | |
| | | | | | 1999) | |
| EMT021 | Silicon | MSPHPHPRHHHTGGGGM | 30 | 3127.4165 | (NAIK; | SEQ ID |
| | | HGKTQATSGTIQS | | | BROTT; | NO: 10 |
| EMT022 | Silicon | RGRRRRLSCRLLGGGGM | 30 | 3198.6687 | CARSON; | SEQ ID |
| | | HGKTQATSGTIQS | | | AL., | NO: 11 |
| | | | | | 2012) | |
| EMT023 | Silicon | DSARGFKKPGKRGGGG | 30 | 3003.3374 | (COYLE, | SEQ ID |
| | | MHGKTQATSGTIQS | | | BANEYX, | NO: 12 |
| | | | | | 2016) | |
| EMT024 | Silicon | HPPMNASHPHMHGGGG | 30 | 3049.3582 | (ETESHOLA; | SEQ ID |
| | | MHGKTQATSGTIQS | | | BRILLSON; | NO: 13 |
| | | | | | LEE, | |
| | | | | | 2005) | |
| EMT025 | Silicon | HKDHHANQHVHMGGGG | 30 | 3147.4045 | (OKAMOTO; | SEQ ID |
| | | MHGKTQATSGTIQS | | | IWAHORI; | NO: 14 |
| | | | | | YAMASHITA, | |
| | | | | | 2019) | |
| EMT026 | Silicon | HPPMNASHPHMHGGGG | 16 | | | SEQ ID |
| | | | | | | NO: 35 |
| Cellulose | Cellulose | PTTGSCAVTYTANGWSG | 108 | | | SEQ ID |
| binding | | GFTAAVTLTNTGTTALSG | | | | NO: 15 |
| motif 1 | | WTLGFAFPSGQTLTQGW | | | | |
| | | SARWAQSGSSVTATNEA | | | | |
| | | WNAVLAPGASVEIGFSG | | | | |
| | | THTGTNTAPATFTVGGA | | | | |
| | | TCTTR | | | | |
| Cellulose | Cellulose | SGPAGCQVLWGVNQWN | 108 | | | SEQ ID |
| binding | | TGFTANVTVKNTSSAPV | | | | NO: 16 |
| motif 2 | | DGWTLTFSFPSGQQVTQ | | | | |
| | | AWSSTVTQSGSAVTVRN | | | | |
| | | APWNGSIPAGGTAQFGF | | | | |
| | | NGSHTGTNAAPTAFSLN | | | | |
| | | GTPCTVG | | | | |
| Polystyrene | Polystyrene | RAFIASRRIRRP | 12 | | | SEQ ID |
| binding | | | | | | NO: 17 |
| motif 1 | | | | | | |
| Polystyrene | Polystyrene | RIIIRRIRR | 9 | | | SEQ ID |
| binding | | | | | | NO: 18 |
| motif 2 | | | | | | |
| Silica | Silica | RGRRRRLSCRLL | 12 | | | SEQ ID |
| Binding | | | | | | NO: 25 |
| Motif 1 | | | | | | |
| Silica | Silica | SSKKSGSYSGSKGSRRIL | 18 | | | SEQ ID |
| Binding | | | | | | NO: 36 |
| Motif 2 | | | | | | |
| Silica | Silica | MSPHPHPRHHHT | 12 | | | SEQ ID |
| Binding | | | | | | NO: 37 |
| Motif 3 | | | | | | |
| Silica | Silica | DSARGFKKPGKR | 12 | | | SEQ ID |
| Binding | | | | | | NO: 38 |
| Motif 4 | | | | | | |
| Silica | Silica | HPPMNASHPHMH | 12 | | | SEQ ID |
| Binding | | | | | | NO: 39 |
| Motif 5 | | | | | | |
| Silica | Silica | HKDHHANQHVHM | 12 | | | SEQ ID |
| Binding | | | | | | NO: 40 |
| Motif 6 | | | | | | |
| Silver | Silver | NPSSLFRYLPSD | 12 | | | SEQ ID |
| Binding | | | | | | NO: 41 |
| Motif 1 | | | | | | |
| Graphene | Graphene | HSSYWYAFNNKT | 12 | | | SEQ ID |
| Binding | | | | | | NO: 42 |
| Motif | | | | | | |
| Zinc | Zinc Oxide | EAHVMHKVAPRP | 12 | | | SEQ ID |
| Oxide | | | | | | NO: 43 |
| Binding | | | | | | |
| Motif 1 | | | | | | |
| Zinc | Zinc Oxide | HHGHSPTSPQVR | 12 | | | SEQ ID |
| Oxide | | | | | | NO: 44 |
| Binding | | | | | | |
| Motif 2 | | | | | | |
| PDMS | PDMS | MVMPGDNIKMVVTLIHPI | 30 | | | SEQ ID |
| Binding | (Polydimethyl- | AMDDGLRFAIRE | | | | NO: 45 |
| Motif 1 | siloxane) | | | | | |
| PDMS | PDMS | VGPNNVPYIVATITSNSA | 45 | | | SEQ ID |
| Binding | (Polydimethyl- | GGQPVSLANLKAMYSIA | | | | NO: 46 |
| Motif 2 | siloxane) | KKYDIPVVMD | | | | |
| PDMS | PDMS | NNSWTRVAFAGLKFQDV | 31 | | | SEQ ID |
| Binding | (Polydimethyl- | GSFDYGRNYGVVYD | | | | NO: 47 |
| Motif 3 | siloxane) | | | | | |
| Gold | Gold 1 | QVQLVESGAEVKKPGES | 120 | | | SEQ ID |
| binding | | LKISCKGSGYSFPSYWIN | | | | NO: 48 |
| motif 1 | | WVRQMPGKGLEWMGMI | | | | |
| | | YPADSDTRYSPSFQGHVT | | | | |
| | | ISADKSINTAYLQWAGLK | | | | |
| | | ASDTAIYYCARLGIGGRY | | | | |
| | | MSRWGQGTLVTVSSA | | | | |

TABLE 1a
SBP Fusion Sequences

| | | | | |
|---|---|---|---|---|
| EMT-03 | Fusion of | MTYKLILNGKTLKGETT | 314 | SEQ ID |
| (ISBP | Protein G to | TEAVDAATAEKVFKQY | | NO: 20 |
| plus | linker to | ANDNGVDGEWTYDDA | | |
| linker) | Gold Protein | TKTFTVTEKPEVIDASEL | | |
| | | TPAVTTYKLVINGKTLK | | |
| | | GETTTEAVDAATAEKVF | | |
| | | KQYANDNGVDGEWTY | | |
| | | DDATKTFTVTEKPEVID | | |
| | | ASELTPAVTTYKLVING | | |
| | | KTLKGETTTKAVDAETA | | |
| | | EKAFKQYANDNGVDGV | | |
| | | WTYDDATKTFTVTEGG | | |
| | | GGSGGGGSGGGGSASG | | |
| | | GGMHGKTQATSGTIQS | | |
| | | MHGKTQATSGTIQSMH | | |
| | | GKTQATSGTIQSMHGKT | | |
| | | QATSGTIQSMHGKTQAT | | |
| | | SGTIQSMHGKTQATSGT | | |
| | | IQSMHGKTQATSGTIQS | | |
| EMT- | Fusion of | MTYKLILNGKTLKGETT | 228 | SEQ ID |
| 012 | Protein G to | TEAVDAATAEKVFKQY | | NO: 49 |
| | linker to | ANDNGVDGEWTYDDA | | |
| | silica | TKTFTVTEKPEVIDASEL | | |
| | binding | TPAVTTYKLVINGKTLK | | |
| | peptide | GETTTEAVDAATAEKVF | | |
| | | KQYANDNGVDGEWTY | | |
| | | DDATKTFTVTEKPEVID | | |
| | | ASELTPAVTTYKLVING | | |
| | | KTLKGETTTKAVDAETA | | |
| | | EKAFKQYANDNGVDGV | | |
| | | WTYDDATKTFTVTEGG | | |
| | | GGSGGGGSGGGGSASG | | |
| | | GGHPPMNASHPHMH | | |
| EMT- | Silica | HPPMNASHPHMHGGGG | 16 | SEQ ID |
| 026 | Binding | | | NO: 50 |
| | Motif 5 with | | | |
| | a | | | |
| | spacer/linker | | | |
| EMT- | Fusion of | MDPSKDSKAQVSAAEA | 278 | SEQ ID |
| 027 | streptavidin | GITGTWYNQLGSTFIVT | | NO: 23 |
| | to linker to | AGADGALTGTYESAVG | | |
| | Gold Protein | NAESRYVLTGRYDSAPA | | |
| | (98 aa Gold | TDGSGTALGWTVAWKN | | |
| | protein) | NYRNAHSATTWSGQYV | | |
| | | GGAEARINTQWLLTSGT | | |
| | | TEANAWKSTLVGHDTF | | |
| | | TKVKPSAASIDAAKKAG | | |
| | | VNNGNPLDAVQQGGGG | | |
| | | SGGGGSGGGGSASGGG | | |
| | | MHGKTQATSGTIQSMH | | |
| | | GKTQATSGTIQSMHGKT | | |
| | | QATSGTIQSMHGKTQAT | | |
| | | SGTIQSMHGKTQATSGT | | |
| | | IQSMHGKTQATSGTIQS | | |
| | | MHGKTQATSGTIQS | | |
| EMT- | Fusion of | MDPSKDSKAQVSAAEA | 188 | SEQ ID |
| 028 | streptavidin | GITGTWYNQLGSTFIVT | | NO: 24 |
| | to linker to | AGADGALTGTYESAVG | | |
| | Gold Protein | NAESRYVLTGRYDSAPA | | |
| | (8 aa Gold | TDGSGTALGWTVAWKN | | |
| | protein) | NYRNAHSATTWSGQYV | | |
| | | GGAEARINTQWLLTSGT | | |
| | | TEANAWKSTLVGHDTF | | |
| | | TKVKPSAASIDAAKKAG | | |
| | | VNNGNPLDAVQQGGGG | | |
| | | SGGGGSGGGGSASGGG | | |
| | | VSGSSPDS | | |
| EMT- | Fusion of | MDPSKDSKAQVSAAEA | 192 | SEQ ID |
| 029 | streptavidin | GITGTWYNQLGSTFIVT | | NO: 26 |
| | (SEQID NO: | AGADGALTGTYESAVG | | |
| | 22) to linker | NAESRYVLTGRYDSAPA | | |
| | (SEQ ID | TDGSGTALGWTVAWKN | | |
| | NO: 1) to | NYRNAHSATTWSGQYV | | |
| | Silica | GGAEARINTQWLLTSGT | | |
| | Binding | TEANAWKSTLVGHDTF | | |
| | Motif | TKVKPSAASIDAAKKAG | | |
| | | VNNGNPLDAVQQGGGG | | |
| | | SGGGGSGGGGSASGGG | | |
| | | RGRRRRLSCRLL | | |
| EMT- | Fusion of | MDPSKDSKAQVSAAEA | | SEQ-ID |
| 030 | Streptavidin | GITGTWYNQLGSTFIVT | | NO: 51 |
| | to spacer to | AGADGALTGTYESAVG | | |
| | Silica | NAESRYVLTGRYDSAPA | | |
| | Binding | TDGSGTALGWTVAWKN | | |
| | Motif | NYRNAHSATTWSGQYV | | |
| | | GGAEARINTQWLLTSGT | | |
| | | TEANAWKSTLVGHDTF | | |
| | | TKVKPSAASIDAAKKAG | | |
| | | VNNGNPLDAVQQGGGG | | |
| | | SGGGGSGGGGSASGGG | | |
| | | SSKKSGSYYSYGTKKSG | | |
| | | SYSGYSTKKSASRRILSS | | |
| | | KKSGSYSGYSTKKSGSR | | |
| | | RILSSKKSGSYSGSKGSK | | |
| | | RRILSSKKSGSYSGSKGS | | |
| | | KRRNLSSKKSGSYSGSK | | |
| | | GSKRRILSSKKSGSYSGS | | |
| | | KGSKRRNLSSKKSGSYS | | |
| | | GSKGSKRRILSGGLRGS | | |
| | | M | | |
| EMT- | Fusion of | MDPSKDSKAQVSAAEA | | SEQ ID |
| 031 | Streptavidin | GITGTWYNQLGSTFIVT | | NO: 52 |
| | to spacer to | AGADGALTGTYESAVG | | |
| | Silver | NAESRYVLTGRYDSAPA | | |
| | Binding | TDGSGTALGWTVAWKN | | |
| | Motif | NYRNAHSATTWSGQYV | | |
| | | GGAEARINTQWLLTSGT | | |
| | | TEANAWKSTLVGHDTF | | |
| | | TKVKPSAASIDAAKKAG | | |
| | | VNNGNPLDAVQQGGGG | | |
| | | SGGGGSGGGGSASGGG | | |
| | | NPSSLFRYLPSD | | |
| EMT- | Fusion of | MDPSKDSKAQVSAAEA | 288 | SEQ ID |
| 032 | streptavidin | GITGTWYNQLGSTFIVT | | NO: 27 |
| | (SEQID NO: | AGADGALTGTYESAVG | | |
| | 22) to linker | NAESRYVLTGRYDSAPA | | |
| | (SEQ ID | TDGSGTALGWTVAWKN | | |
| | NO: 1) to | NYRNAHSATTWSGQYV | | |
| | Cellulose | GGAEARINTQWLLTSGT | | |
| | binding | TEANAWKSTLVGHDTF | | |
| | motif 1 | TKVKPSAASIDAAKKAG | | |
| | | VNNGNPLDAVQQGGGG | | |
| | | SGGGGSGGGGSASGGG | | |
| | | PTTGSCAVTYTANGWS | | |
| | | GGFTAAVTLTNTGTTAL | | |
| | | SGWTLGFAFPSGQTLTQ | | |
| | | GWSARWAQSGSSVTAT | | |
| | | NEAWNAVLAPGASVEI | | |
| | | GFSGTHTGTNTAPATFT | | |
| | | VGGATCTTR | | |
| EMT- | Fusion of | MDPSKDSKAQVSAAEA | 288 | SEQ ID |
| 033 | streptavidin | GITGTWYNQLGSTFIVT | | NO: 28 |
| | (SEQID NO: | AGADGALTGTYESAVG | | |
| | 22) to linker | NAESRYVLTGRYDSAPA | | |
| | (SEQ ID | TDGSGTALGWTVAWKN | | |
| | NO: 1) to | NYRNAHSATTWSGQYV | | |
| | Cellulose | GGAEARINTQWLLTSGT | | |
| | binding | TEANAWKSTLVGHDTF | | |
| | motif 2 | TKVKPSAASIDAAKKAG | | |
| | | VNNGNPLDAVQQGGGG | | |
| | | SGGGGSGGGGSASGGG | | |
| | | SGPAGCQVLWGVNQW | | |
| | | NTGFTANVTVKNTSSAP | | |
| | | VDGWTLTFSFPSGQQVT | | |
| | | QAWSSTVTQSGSAVTV | | |
| | | RNAPWNGSIPAGGTAQF | | |
| | | GFNGSHTGTNAAPTAFS | | |
| | | LNGTPCTVG | | |
| GL008 | Fusion of | MDPSKDSKAQVSAAEA | 192 | SEQ ID |
| | streptavidin | GITGTWYNQLGSTFIVT | | NO: 30 |
| | (SEQID NO: | AGADGALTGTYESAVG | | |
| | 22) to linker | NAESRYVLTGRYDSAPA | | |
| | (SEQ ID | TDGSGTALGWTVAWKN | | |
| | NO: 1) to | NYRNAHSATTWSGQYV | | |
| | Polystyrene | GGAEARINTQWLLTSGT | | |
| | binding | TEANAWKSTLVGHDTF | | |
| | motif 1 | TKVKPSAASIDAAKKAG | | |
| | | VNNGNPLDAVQQGGGG | | |
| | | SGGGGSGGGGSASGGG | | |
| | | RAFIASRRIRRP | | |
| GL009 | Fusion of | MDPSKDSKAQVSAAEA | 189 | SEQ ID |
| | streptavidin | GITGTWYNQLGSTFIVT | | NO: 31 |
| | (SEQID NO: | AGADGALTGTYESAVG | | |
| | 22) to linker | NAESRYVLTGRYDSAPA | | |
| | (SEQ ID | TDGSGTALGWTVAWKN | | |
| | NO: 1) to | NYRNAHSATTWSGQYV | | |
| | Polystyrene | GGAEARINTQWLLTSGT | | |
| | binding | TEANAWKSTLVGHDTF | | |
| | motif 2 | TKVKPSAASIDAAKKAG | | |
| | | VNNGNPLDAVQQGGGG | | |
| | | SGGGGSGGGGSASGGG | | |
| | | RIIIRRIRR | | |
| GL010 | Fusion of | GDPSKDSKAQVSAAEA | 192 | SEQ ID |
| | streptavidin | GITGTWYNQLGSTFIVT | | NO: 53 |
| | to linker to | AGADGALTGTYESAVG | | |
| | Graphene | NAESRYVLTGRYDSAPA | | |
| | Binding | TDGSGTALGWTVAWKN | | |
| | Motif | NYRNAHSATTWSGQYV | | |
| | | GGAEARINTQWLLTSGT | | |
| | | TEANAWKSTLVGHDTF | | |
| | | TKVKPSAASIDAAKKAG | | |
| | | VNNGNPLDAVQQGGGG | | |
| | | SGGGGSGGGGSASGGG | | |
| | | HSSYWYAFNNKT | | |
| GL011 | Affinity tag | MHHHHHHENLYFQGDP | 291 | SEQ ID |
| | (his-tag)- | SKDSKAQVSAAEAGITG | | NO: 34 |
| | Fusion of | TWYNQLGSTFIVTAGAD | | |
| | streptavidin | GALTGTYESAVGNAESR | | |
| | to linker | YVLTGRYDSAPATDGSG | | |
| | (SEQ ID | TALGWTVAWKNNYRN | | |
| | NO:1) to | AHSATTWSGQYVGGAE | | |
| | Gold Protein | ARINTQWLLTSGTTEAN | | |
| | (98 aa Gold | AWKSTLVGHDTFTKVK | | |
| | protein) | PSAASIDAAKKAGVNNG | | |
| | | NPLDAVQQGGGGSGGG | | |
| | | GSGGGGSASGGGMHGK | | |
| | | TQATSGTIQSMHGKTQA | | |
| | | TSGTIQSMHGKTQATSG | | |
| | | TIQSMHGKTQATSGTIQ | | |
| | | SMHGKTQATSGTIQSMH | | |
| | | GKTQATSGTIQSMHGKT | | |
| | | QATSGTIQS | | |
| GL012 | Affinity tag | MHHHHHHENLYFQGDP | 205 | SEQ ID |
| | (his-tag)- | SKDSKAQVSAAEAGITG | | NO: 54 |
| | Fusion of | TWYNQLGSTFIVTAGAD | | |
| | streptavidin | GALTGTYESAVGNAESR | | |
| | to linker to | YVLTGRYDSAPATDGSG | | |
| | ZnO binding | TALGWTVAWKNNYRN | | |
| | motif | AHSATTWSGQYVGGAE | | |
| | | ARINTQWLLTSGTTEAN | | |
| | | AWKSTLVGHDTFTKVK | | |
| | | PSAASIDAAKKAGVNNG | | |
| | | NPLDAVQQGGGGSGGG | | |
| | | GSGGGGSASGGGEAHV | | |
| | | MHKVAPRP | | |
| GL013 | Affinity tag | MHHHHHHENLYFQGDP | 205 | SEQ ID |
| | (his-tag)- | SKDSKAQVSAAEAGITG | | NO: 55 |
| | Fusion of | TWYNQLGSTFIVTAGAD | | |
| | streptavidin | GALTGTYESAVGNAESR | | |
| | to linker) to | YVLTGRYDSAPATDGSG | | |
| | ZnO binding | TALGWTVAWKNNYRN | | |
| | motif | AHSATTWSGQYVGGAE | | |
| | | ARINTQWLLTSGTTEAN | | |
| | | AWKSTLVGHDTFTKVK | | |
| | | PSAASIDAAKKAGVNNG | | |
| | | NPLDAVQQGGGGSGGG | | |
| | | GSGGGGSASGGGHHGH | | |
| | | SPTSPQVR | | |
| GL014 | Affinity tag | MHHHHHHENLYFQGDP | 223 | SEQ ID |
| | (his-tag)- | SKDSKAQVSAAEAGITG | | NO: 56 |
| | Fusion of | TWYNQLGSTFIVTAGAD | | |
| | streptavidin | GALTGTYESAVGNAESR | | |
| | to linker to | YVLTGRYDSAPATDGSG | | |
| | PDMS | TALGWTVAWKNNYRN | | |
| | binding | AHSATTWSGQYVGGAE | | |
| | motif | ARINTQWLLTSGTTEAN | | |
| | | AWKSTLVGHDTFTKVK | | |
| | | PSAASIDAAKKAGVNNG | | |
| | | NPLDAVQQGGGGSGGG | | |
| | | GSGGGGSASGGGMVMP | | |
| | | GDNIKMVVTLIHPIAMD | | |
| | | DGLRFAIRE | | |
| GL015 | Affinity tag | MHHHHHHENLYFQGDP | 238 | SEQ ID |
| | (his-tag)- | SKDSKAQVSAAEAGITG | | NO: 57 |
| | Fusion of | TWYNQLGSTFIVTAGAD | | |
| | streptavidin | GALTGTYESAVGNAESR | | |
| | to linker to | YVLTGRYDSAPATDGSG | | |
| | PDMS | TALGWTVAWKNNYRN | | |
| | binding | AHSATTWSGQYVGGAE | | |
| | motif | ARINTQWLLTSGTTEAN | | |
| | | AWKSTLVGHDTFTKVK | | |
| | | PSAASIDAAKKAGVNNG | | |
| | | NPLDAVQQGGGGSGGG | | |
| | | GSGGGGSASGGGVGPN | | |
| | | NVPYIVATITSNSAGGQP | | |
| | | VSLANLKAMYSIAKKY | | |
| | | DIPVVMD | | |
| GL016 | Affinity tag | MHHHHHHENLYFQGDP | 224 | SEQ ID |
| | (his-tag)- | SKDSKAQVSAAEAGITG | | NO: 58 |
| | Fusion of | TWYNQLGSTFIVTAGAD | | |
| | streptavidin | GALTGTYESAVGNAESR | | |
| | to linker to | YVLTGRYDSAPATDGSG | | |
| | PDMS | TALGWTVAWKNNYRN | | |
| | binding | AHSATTWSGQYVGGAE | | |
| | motif | ARINTQWLLTSGTTEAN | | |
| | | AWKSTLVGHDTFTKVK | | |
| | | PSAASIDAAKKAGVNNG | | |
| | | NPLDAVQQGGGGSGGG | | |
| | | GSGGGGSASGGGNNSW | | |
| | | TRVAFAGLKFQDVGSFD | | |
| | | YGRNYGVVYD | | |
| GL017 | Affinity tag | MHHHHHHENYLFQGQV | 315 | SEQ ID |
| | (his-tag)- | QLVESGAEVKKPGESLK | | NO: 59 |
| | Fusion of | ISCKGSGYSFPSYWINW | | |
| | ScFv to | VRQMPGKGLEWMGMI | | |
| | linker to | YPADSDTRYSPSFQGHV | | |
| | streptavidin | TISADKSINTAYLQWAG | | |
| | | LKASDTAIYYCARLGIG | | |
| | | GRYMSRWGQGTLVTVS | | |
| | | SAPTPTPTTPTPTPTTPTP | | |
| | | TPSTDPSKDSKAQVSAA | | |
| | | EAGITGTWYNQLGSTFI | | |
| | | VTAGADGALTGTYESA | | |
| | | VGNAESRYVLTGRYDS | | |
| | | APATDGSGTALGWTVA | | |
| | | WKNNYRNAHSATTWSG | | |
| | | QYVGGAEARINTQWLL | | |
| | | TSGTTEANAWKSTLVG | | |
| | | HDTFTKVKPSAASIDAA | | |
| | | KKAGVNNGNPLDAVQQ | | |
| GL018 | Affinity tag | MHHHHHHENLYFQGDP | 302 | SEQ ID |
| | (his-tag)- | SKDSKAQVSAAEAGITG | | NO: 62 |
| | TEV | TWYNQLGSTFIVTAGAD | | |
| | cleavage | GALTGTYESAVGNAESR | | |
| | site- Fusion | YVLTGRYDSAPATDGSG | | |
| | of | TALGWTVAWKNNYRN | | |
| | streptavidin | AHSATTWSGQYVGGAE | | |
| | (SEQ ID | ARINTQWLLTSGTTEAN | | |
| | NO: 60) to | AWKSTLVGHDTFTKVK | | |
| | linker (SEQ | PSAASIDAAKKAGVNNG | | |
| | ID NO: 61) | NPLDAVQQPTPTPTTPTP | | |
| | to Cellulose | TPTTPTPTPSTSGPAGCQ | | |
| | binding | VLWGVNQWNTGFTAN | | |
| | motif 2 | VTVKNTSSAPVDGWTL | | |
| | (SEQ ID | TFSFPSGQQVTQAWSST | | |
| | NO: 18) | VTQSGSAVTVRNAPWN | | |
| | | GSIPAGGTAQFGFNGSH | | |
| | | TGTNAAPTAFSLNGTPC | | |
| | | TVG | | |
| GL019 | Affinity tag | MHHHHHHENLYFQGSG | 301 | SEQ ID |
| | (his-tag)- | PAGCQVLWGVNQWNT | | NO: 63 |
| | TEV | GFTANVTVKNTSSAPVD | | |
| | cleavage | GWTLTFSFPSGQQVTQA | | |
| | site- Fusion | WSSTVTQSGSAVTVRN | | |
| | of Cellulose | APWNGSIPAGGTAQFGF | | |
| | binding | NGSHTGTNAAPTAFSLN | | |
| | motif 2 | GTPCTVGGGGGSGGGG | | |
| | (SEQ ID | SGGGGSASGGGDPSKDS | | |
| | NO: 18) to | KAQVSAAEAGITGTWY | | |
| | linker (SEQ | NQLGSTFIVTAGADGAL | | |
| | ID NO: 1) to | TGTYESAVGNAESRYVL | | |
| | streptavidin | TGRYDSAPATDGSGTAL | | |
| | (SEQ ID | GWTVAWKNNYRNAHS | | |
| | NO: 60) | ATTWSGQYVGGAEARI | | |
| | | NTQWLLTSGTTEANAW | | |
| | | KSTLVGHDTFTKVKPSA | | |
| | | ASIDAAKKAGVNNGNP | | |
| | | LDAVQQ | | |
| GL020 | Affinity tag | MHHHHHHENLYFQGSG | 302 | SEQ ID |
| | (his-tag)- | PAGCQVLWGVNQWNT | | NO: 64 |
| | TEV | GFTANVTVKNTSSAPVD | | |
| | cleavage | GWTLTFSFPSGQQVTQA | | |
| | site- Fusion | WSSTVTQSGSAVTVRN | | |
| | of Cellulose | APWNGSIPAGGTAQFGF | | |
| | binding | NGSHTGTNAAPTAFSLN | | |
| | motif 2 | GTPCTVGPTPTPTTPTPT | | |
| | (SEQ ID | PTTPTPTPSTDPSKDSKA | | |
| | NO: 18) to | QVSAAEAGITGTWYNQ | | |
| | linker (SEQ | LGSTFIVTAGADGALTG | | |
| | ID NO: 61) | TYESAVGNAESRYVLTG | | |
| | to | RYDSAPATDGSGTALG | | |
| | streptavidin | WTVAWKNNYRNAHSA | | |
| | (SEQ ID | TTWSGQYVGGAEARIN | | |
| | NO: 60) | TQWLLTSGTTEANAWK | | |
| | | STLVGHDTFTKVKPSAA | | |
| | | SIDAAKKAGVNNGNPL | | |
| | | DAVQQ | | |

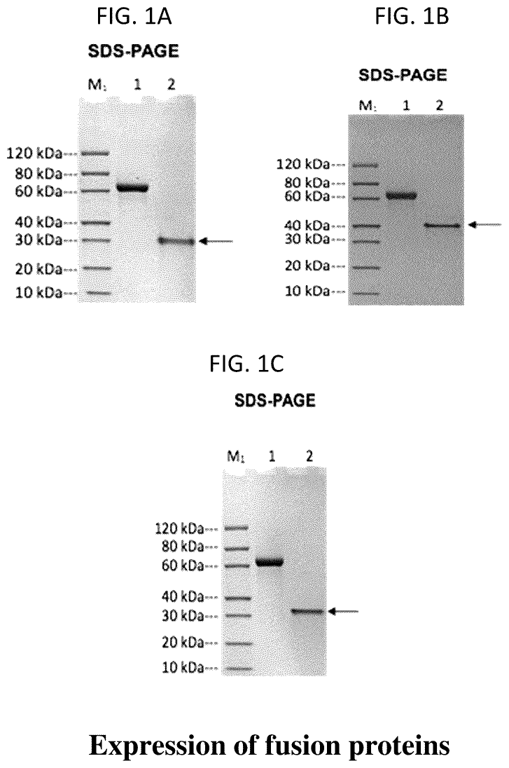

Purities of 90% were achieved for the fusion protein and the ISBP-free G′ proteins, as shown in FIG. 1. In FIG. 1, three gels A), B) and C) show the expression and purity of the gold-binding and silica-binding fusion proteins on Coomassie-stained SDS-PAGE gels. 2 μg of BSA was added in lane 1 of each gel A), B) and C) as a loading control. Gel A) shows an ISBP-free fusion protein, Gel B) shows a full Gold-binding fusion protein, and Gel C) shows a full Silica-binding fusion protein.

### EXAMPLE 3

Functionalizing the QCM-D gold sensor with gold-binding fusion protein, and testing using the SARS-CoV-2 Spike protein antibody antigen system: Sensor chips were prepared and equilibrated in PBS as described above. Samples were diluted to 50 μg/mL using 10 mM PBS. The gold-binding fusion protein from Example 2 at 50 μg/mL in PBS was flowed over the sensor chips at 50 μL/min until Δfₙ equilibrated, after which the sensor chips were washed with PBS followed by a BSA (50 μg/mL PBS) blocking step.

The SARS-CoV-2 Spike protein antibody was then flowed over the sensor chips at 50 μL/min, followed by a PBS wash step, and then finally the SARS-CoV-2 Spike antigen (50 μg/mL) or the negative control (SARS-CoV-2 Nucleocapsid antigen, 50 μg/mL) were flowed until the samples were consumed. The sensors were washed with PBS buffer to eliminate nonspecific binding. The raw data was analyzed in Qsense™ Dfind™ analysis software using a Kelvin-Voigt viscoelastic model.

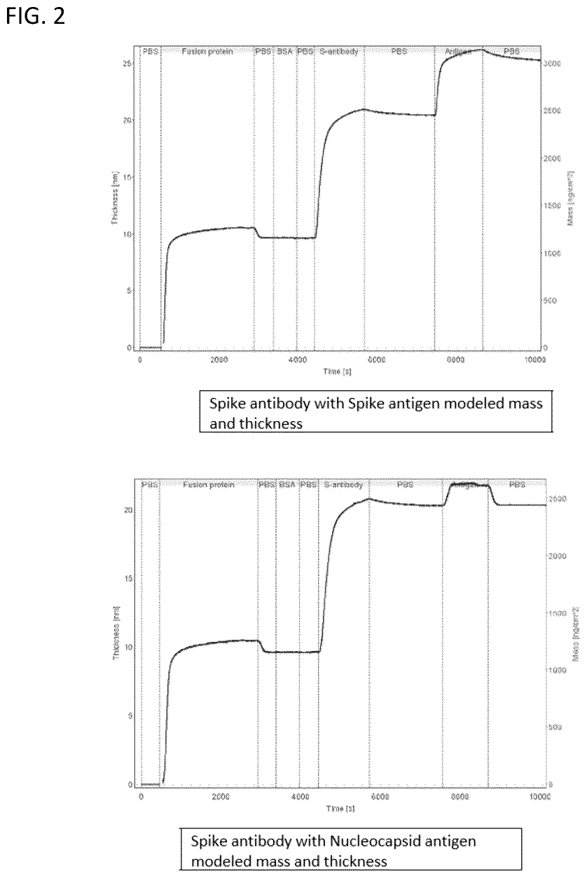

The gold-binding fusion protein was found to bind to the gold sensor surface in two experiments, forming a 10.56 nm and 10.5 nm layer, respectively, with only a very small fraction washed off during the subsequent wash step (remaining layer thickness 9.66 nm and 9.6 nm, respectively). No significant changes to the thickness or mass of the layers occurred during the subsequent blocking with BSA and washing steps. The SARS-CoV-2 Spike protein antibody was then flowed across the biolayer and the thickness and mass of both layers more than doubled. After a second washing with PBS, a biolayer of 20.45 nm (FIG. 2 left) and 20.3 nm (FIG. 2 right) respectively, remained. To test the immobilized antibodies' ability to bind antigens, and their specificity, SARS-CoV-2 Spike antigen (FIG. 2 left) and SARS-CoV-2 Nucleocapsid antigen (FIG. 2 right) were tested in each system. The SARS-CoV-2 Spike antibody immobilized on the gold sensor via the gold-fusion protein appeared to bind the Spike antigen, forming a layer of 25.29 nm after washing with PBS, but not the Nucleocapsid antigen, leaving a layer of only 20.4 nm after the PBS wash (comparable to the antibody-only layer).

### EXAMPLE 4

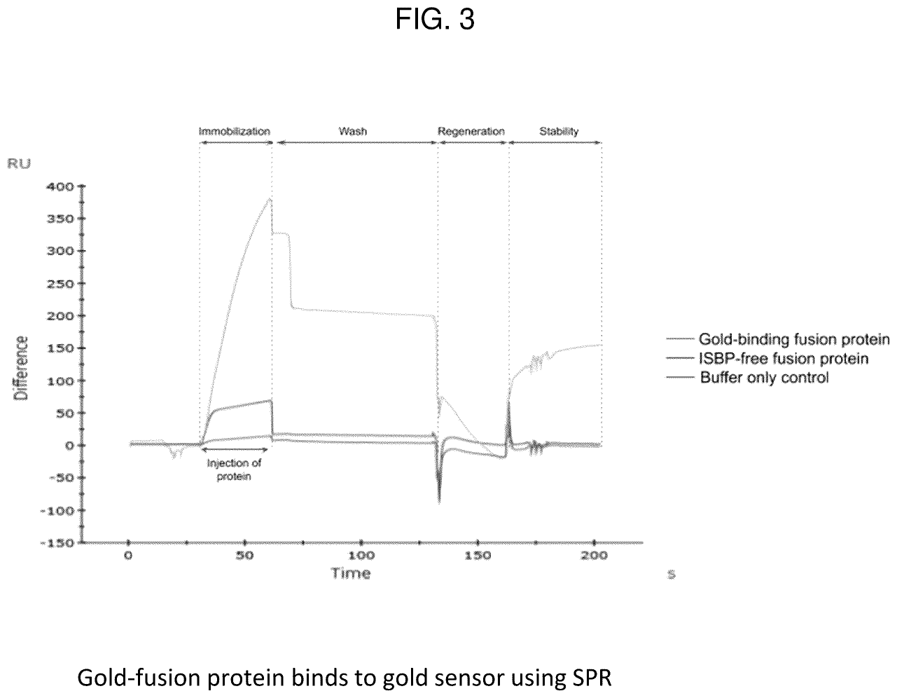

Evaluating the binding kinetics of the SARS-CoV-2 Spike antibody binding to the gold-binding fusion protein, and its ability to bind the Spike antigen: Surface plasmon resonance (SPR), an opto-electronic biosensing technique, was chosen to evaluate the binding kinetics of the Spike antibody to the gold-fusion protein bound to a gold sensor. First, the immobilization of the gold-binding fusion protein and two controls (ISBP-free fusion protein and buffer only) was evaluated. Zero or minimal binding was observed for those controls (FIG. 3). The gold-binding fusion protein, however, showed a five-fold increase in Resonance Units (RU) during the immobilization phase compared to the ISBP-free version. A significant amount of gold-binding protein stayed immobilized on the gold sensor even after the regeneration buffer was injected, indicating that the coated sensor can potentially be reused.

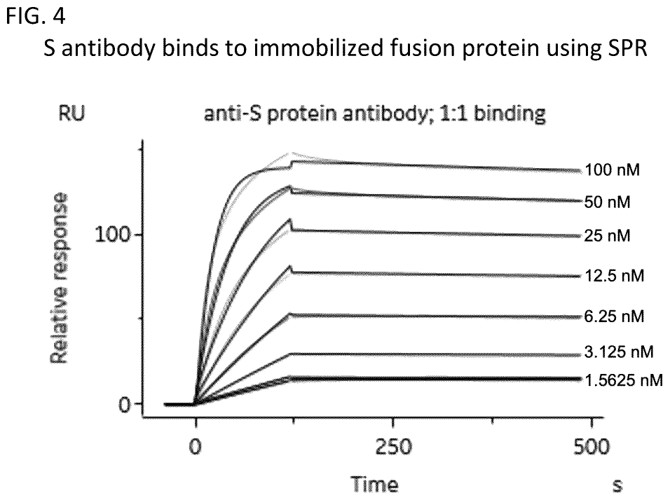

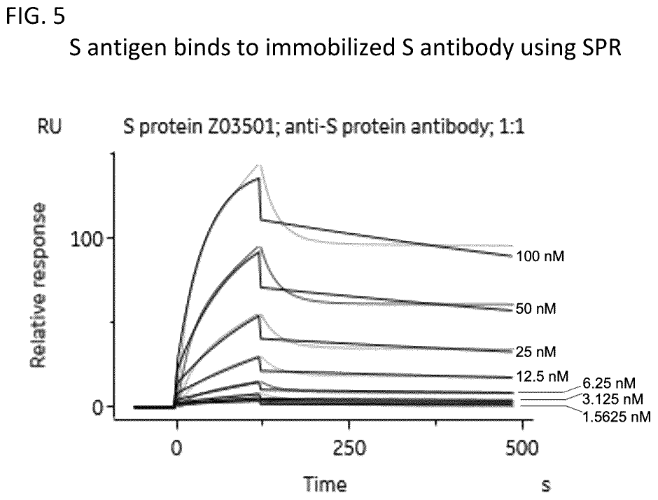

The ability and the binding kinetics of the SARS-CoV-2 Spike and Nucleocapsid antibodies to bind to the gold-binding fusion protein immobilized on the sensor surface, and the respective antigens binding to the antibodies, was tested using a dilution series. Dilution series for both antibodies (FIG. 4) and the Spike antigen (FIG. 5) were performed spanning the following concentrations in two experiments: 1.5625 nM (×2), 3.125 nM, 6.25 nM, 12.5 nM, 25 nM, 50 nM and 100 nM. The best concentration for antibody loading was empirically 2 μg/mL and used as the basis for the antigen dilution series. The raw data was analyzed using Biacore™ 8K Evaluation software version 1.1. As shown in FIG. 4 SARS-CoV-2 Spike antibody binds to the fusion protein. The binding kinetics results for both the SARS-CoV-2 Spike and Nucleocapsid antibody binding to the fusion protein are set out in Table 2.

TABLE 2
Spike and Nucleocapsid Antibodies Bind to
Immobilized Fusion Protein Using SPR.

| | | Chi² | ka | kd | KD | Rmax |
|---|---|---|---|---|---|---|
| Ligand | Pathogen | (RU²) | (1/Ms) | (1/s) | (M) | (RU) |
| Gold fusion | anti-N protein | 1.38 | 5.77 | 1.49 | 2.58 | 104.2 |
| protein | antibody | E+00 | E+05 | E−04 | E−10 | |
| Gold fusion | anti-S protein | 1.97 | 5.37 | 1.03 | 1.92 | 134.4 |
| protein | antibody | E+00 | E+05 | E−04 | E−10 | |

In Table 2, the binding kinetics of the Spike protein antibody and the Nucleocapsid antibody to the gold-binding fusion protein are shown. The kinetics of interaction was calculated and dissociation constants (K_{D}) of 1.92E-10 M and 2.58E-10 M were found for the SARS-CoV-2 Spike and Nucleocapsid antibody, respectively. This compares favorably to the K_{D} levels reported in the literature which show that protein G binds all human IgG subclasses at ˜2E-10 M. As with QCM-D, these results show that the gold-binding fusion proteins efficiently bind to the gold sensor surface, immobilize and orient the SARS-CoV-2 Spike and Nucleocapsid antibodies. The SARS-CoV-2 S protein antigen then also binds to the Spike protein antibody with a K_{D} of 2.39E-9 M, a typical range for a monoclonal antibody/antigen interaction, indicating that the bound Spike protein antibody was able to maintain its antigen binding affinity (FIG. 5). The results are also shown here in Table 3.

TABLE 3
Spike Antigen Binds to Immobilized
Spike Antibody Binding Kinetics

| | | | Chi² | ka | kd | KD | Rmax |
|---|---|---|---|---|---|---|---|
| Ligand | Capture | Pathogen | (RU²) | (1/Ms) | (1/s) | (M) | (RU) |
| Gold | anti-S | S antigen | 7.93 | 2.52 | 6.03 | 2.39 | 167.7 |
| fusion | protein | | E+00 | E+05 | E−04 | E−09 | |
| protein | antibody | | | | | | |

### EXAMPLE 5

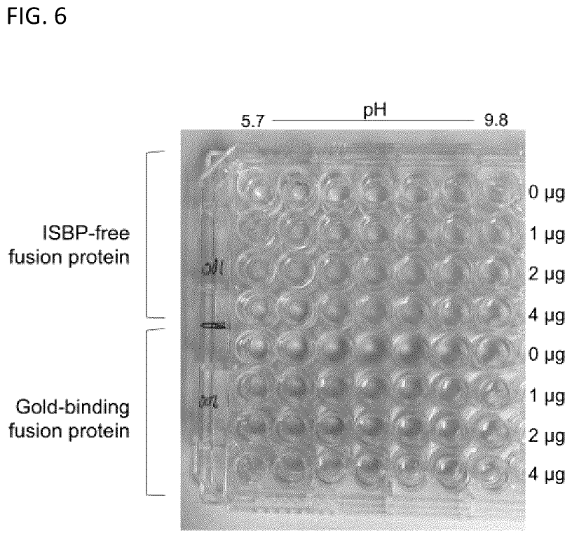

Conjugation of gold-binding fusion proteins to gold nanoparticles: The conjugation of the gold-binding fusion protein and the ISBP-free fusion protein control to 40 nm gold nanoparticles (Cytodiagnostics) was tested in 10 mM PBS buffer using increasing amounts of proteins (0, 1, 2, 4 μg per 100 μL of 1 OD gold) and increasing pH conditions (5.7-9.8). Results are shown in FIG. 6, with the hand drawn section divider separating ISBP-free fusion protein (upper four rows) from gold-binding fusion protein (lower for rows).

Scale-up conjugation reaction for gold-binding fusion protein: The pH of 1 mL 40 nm standard gold nanoparticles was adjusted through the addition of 40 μL of 0.1M sodium phosphate pH 6.5. A 10 μg aliquot of fusion protein was transferred to a separate microcentrifuge vial and diluted to a total volume of 100 μL with ddH₂O. The pH-adjusted gold nanoparticles were rapidly added to the vial of diluted fusion protein and incubated for 30 minutes at room temperature. 50 μL of 10% (w/v) BSA were added to the gold-fusion protein mixture and incubated for 5 minutes to block. The conjugation mixture was centrifuged at 1600×g for 25 minutes and the supernatant removed. Finally, the gold conjugate pellet was resuspended with 1×PBS, 1% BSA to a final concentration of OD=5.5 and stored at 4 degrees until use.

### EXAMPLE 6

Comparative binding analysis of synthetic peptides to gold and silica sensors using QCM-D: Six gold-binding and six silica-binding peptides, described in the literature as binding to gold and silica and depicted in Table 1, were synthesized. Their ability to bind to gold and silica sensors was tested using quartz crystal microbalance with dissipation monitoring (QCM-D). The thickness, the mass deposited, elasticity and viscosity of the resulting layers after a PBS wash were calculated and are summarized in Table 4.

TABLE 4
Comparative binding experiments

| | | | Mass | Molar | Thickness | Elasticity | |
|---|---|---|---|---|---|---|---|
| | | Molecular | after | mass after | after | after | Viscosity |
| | Length | weight | rinse | rinse | rinse | rinse | after rinse |
| Peptide | (aa) | (Da) | (ng/cm²) | (μmol/m²) | (nm) | (kPa) | (mPa-s) |
| EMT014 | 42 | 4303.752 | 372.123 | 0.865 | 2.819 | 133.919 | 3077.028 |
| EMT015 | 98 | 10018.0684 | 654.272 | 0.653 | 5.152 | 313.47 | 4284.443 |
| EMT016 | 12 | 1454.7371 | 441.732 | 3.037 | 3.248 | 111.513 | 1547.205 |
| EMT017 | 10 | 1206.2336 | 560.082 | 4.643 | 4.118 | 147.128 | 1458.707 |
| EMT018 | 10 | 1438.6042 | 333.953 | 2.321 | 2.456 | 105.231 | 1623.188 |
| EMT019 | 8 | 734.716 | 614.052 | 8.358 | 4.482 | 184.355 | 2039.578 |
| EMT020 | 36 | 3541.8909 | 437.373 | 1.235 | 3.289 | 140.22 | 1758.533 |
| EMT021 | 30 | 3127.4165 | 105.78 | 0.338 | 0.789 | 124.66 | 1547.475 |
| EMT022 | 30 | 3198.6687 | 553.397 | 1.73 | 4.13 | 143.679 | 2320.274 |
| EMT023 | 30 | 3003.3374 | 374.014 | 1.245 | 2.791 | 156.079 | 1722.904 |
| EMT024 | 30 | 3049.3582 | 412.778 | 1.354 | 3.08 | 97.644 | 1163.943 |
| EMT025 | 30 | 3147.4045 | 144.062 | 0.458 | 1.075 | 217.69 | 1432.914 |

Table 4 summarizes comparative binding experiments of six gold-binding dual-affinity probes (EMT014-EMT019) and six silica-binding dual-affinity probes (EMT020-EMT025) using quartz crystal microbalance with dissipation monitoring (QCM-D). The mass (ng/cm²), molar mass (μmol/m²), thickness (nm), elasticity (kPa) and viscosity (mPa s) for all peptides is reported.

EMT015, the longest gold-binding peptide, showed the highest mass (ng/cm²) deposited on the gold sensor, while EMT019, the shortest gold-binding peptide showed the highest loading when adjusted for the molecular weight of the peptide (indicated as molar mass (μmol/m²)). The adjusted measurement is a better indicator of the degree of binding. EMT015 built the thickest layer at 5.152 nm with EMT019 the second highest at 4.48 nm. The layer formed with EMT015 also showed higher elasticity and viscosity compared to the other peptides. For the silica-binding peptides, EMT022 showed the highest mass and molar mass deposited onto the silica sensor with a thickness of 4.1 nm compared to the other peptides. It also showed the highest viscosity and second highest elasticity.

### EXAMPLE 7

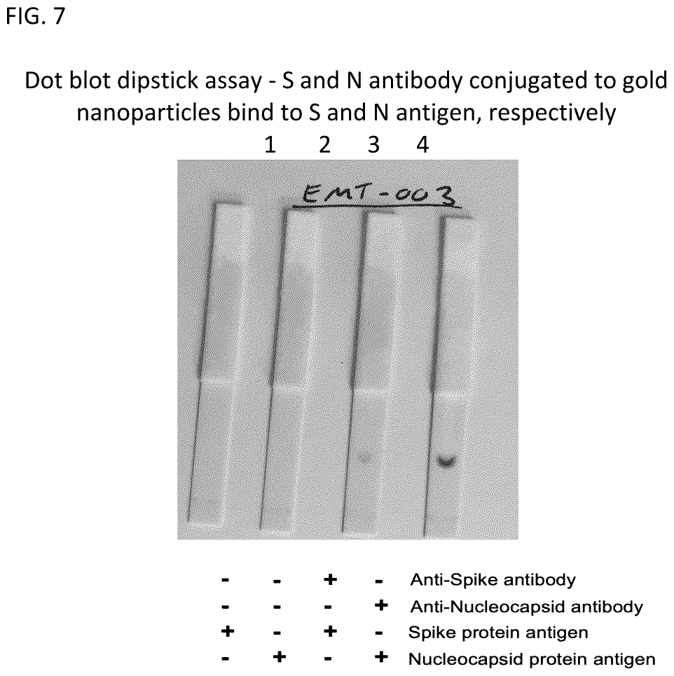

Dot blot dipstick assay: Immobilization of antibodies onto gold-binding fusion protein coated gold nanoparticles and their antigen binding capacity was tested using a dot blot dipstick assay for SARS-CoV-2 Spike and Nucleocapsid antigens. The amount of 0.5 μg of each of S protein and N protein antigen (diluted in 10 mM sodium phosphate buffer, pH 7.4) was spotted on nitrocellulose dip sticks. The dip sticks were then incubated in 80 μL of sample buffer (1×PBS (pH 8), 5% BSA, 0.5% Casein, 0.2% Tween 20, 1% PEG 8000), 10 μL OD 5.5 conjugate (prepared as described above) and 0.135 μg (in 1 μL) of the respective antibodies for 20 minutes at room temperature. The results are shown in the photograph in FIG. 7.

SARS-CoV-2 Spike or Nucleocapsid antibodies conjugated to the gold nanoparticles via the gold-fusion protein proteins were able to bind to the Spike or nucleocapsid antigen spotted onto the dipstick when wicked along the nitrocellulose membrane (Strips 3 and 4). More antibodies seemed to bind to the Nucleocapsid antigen compared to the Spike protein antigen. No signal was detected when only gold nanoparticles with gold-binding fusion conjugates were wicked along the membranes (Strips 1 and 2).

### EXAMPLE 8

Liquid Chromatography-Tandem Mass Spectrometry (LC-MS/MS) Sequence Coverage Analysis:

Proteins are first digested to peptides by appropriate enzymes, such as Trypsin. Then, the peptide mixture is separated by liquid chromatography. Finally, the MS1 and MS2 spectrums of each peptide are detected by mass spectrometry.

Bioanalytical software matches the observed MS1 and MS2 spectrums to theoretical values to identify each peptide of the protein, and then calculates the peptide (or amino acid) coverage rate.

Sample Preparation

A 50 μL protein sample was diluted by 50 mM Tris-HCl to make a final concentration of 0.2 mg/mL. Then, 0.1M DTT was added at 1:20 DTT-to-protein volume ratio to reduce the disulfide bonds. After that, trypsin was added at 1:40 trypsin-to-protein mass ratio for 6 h digestion.

Finally, peptides were dried and re-diluted using 20 μL 0.1% FA-H₂O for UPLC-MS analysis. UPLC Separation:

Column temperature: 50° C., Flow rate: 300 μL/min, Mobile Phase: Solvent A: 0.1% FA-2% ACN in Water, Solvent B: 0.1% FA-90% ACN in Water

Electrospray voltage 3.5 kV, m/z scan range 200-2000 Ion transfer tube temperature, 333° C., AGC 2e5, Resolution of MS120000, Collision energy 32 eV, Resolution of MS/MS 15000; Threshold ion count, 20000 ions/s.

BioPharma™ Finder™ 3.0 was used for LC-MS/MS data analysis. Results: The sequence coverage was 94.0% for the ISBP-free fusion protein (FIG. 1A), 95.85% for the gold-binding fusion protein (FIG. 1B), and 94.3% for silica-binding fusion protein (FIG. 1C). The sequence coverage merely indicates what proportion of the protein was sequenced using the LC-MS/MS method. The LC-MS/MS analysis confirms the amino acid sequence of the proteins and indicates that the ISBP-fusion protein, the gold-binding fusion protein and the silica-binding fusion protein were expressed as expected.

### EXAMPLE 9

Comparative Binding Analysis Evaluating the Direct Binding of Gold-Fusion Protein onto a Gold Sensor Versus Traditional EDC-NHS Conjugation onto a Gold Sensor.

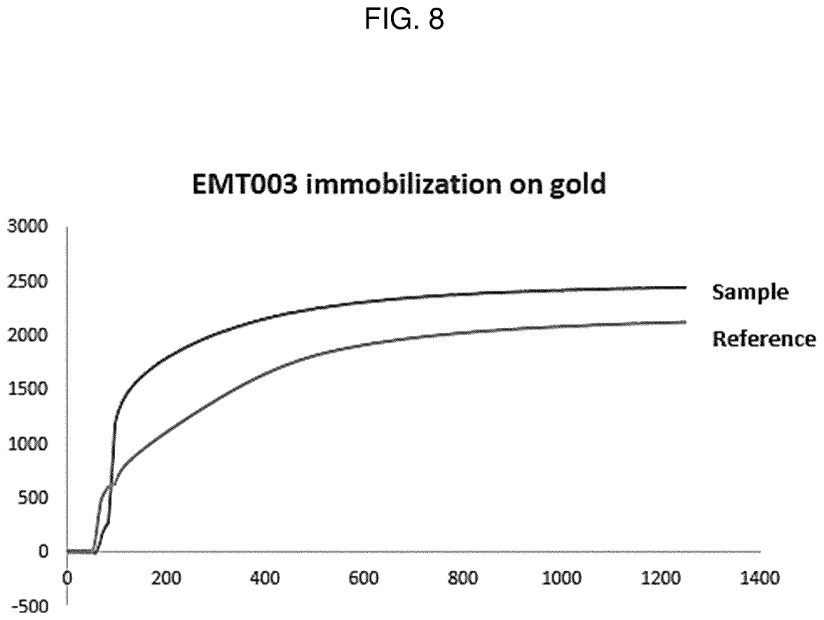

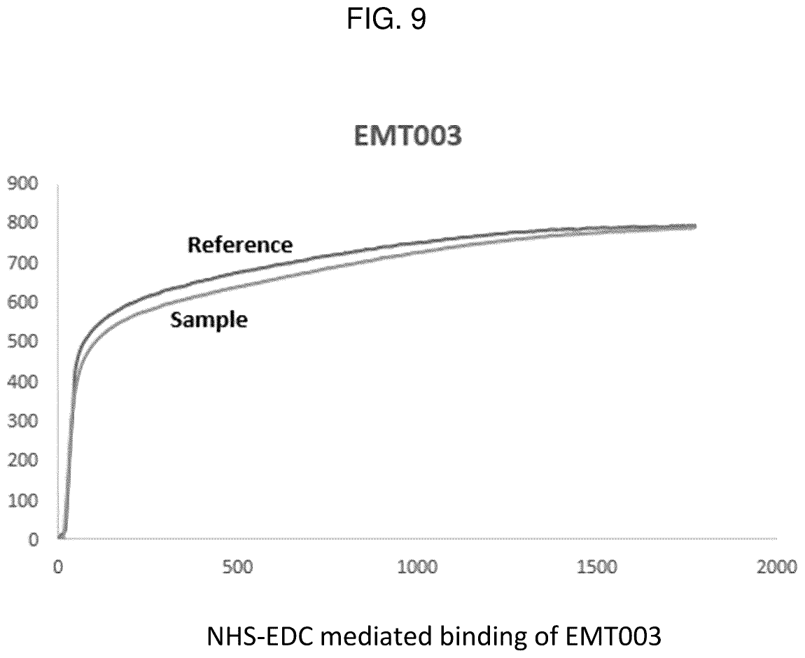

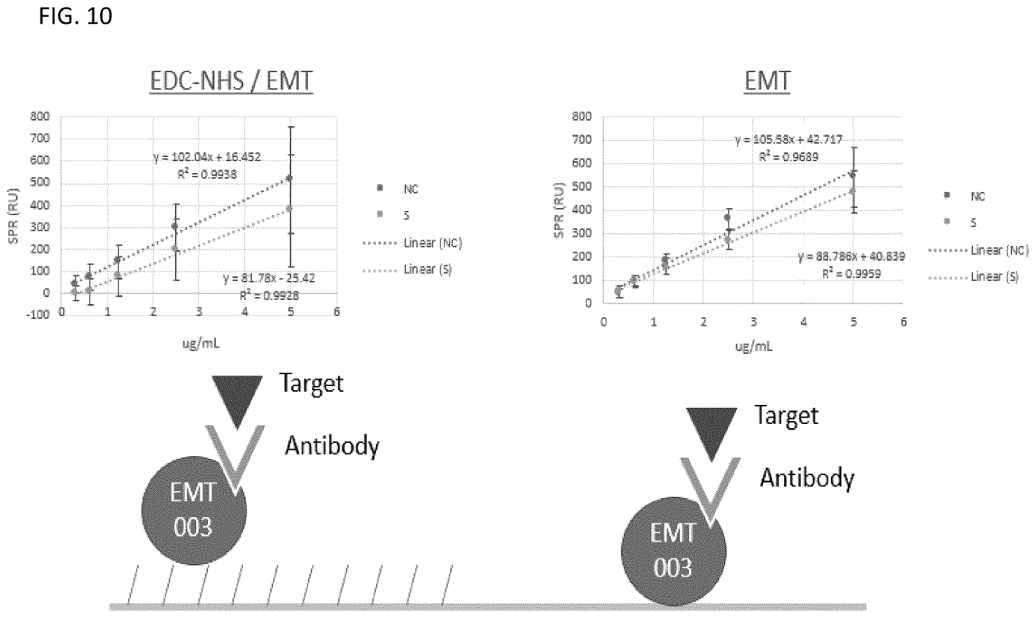

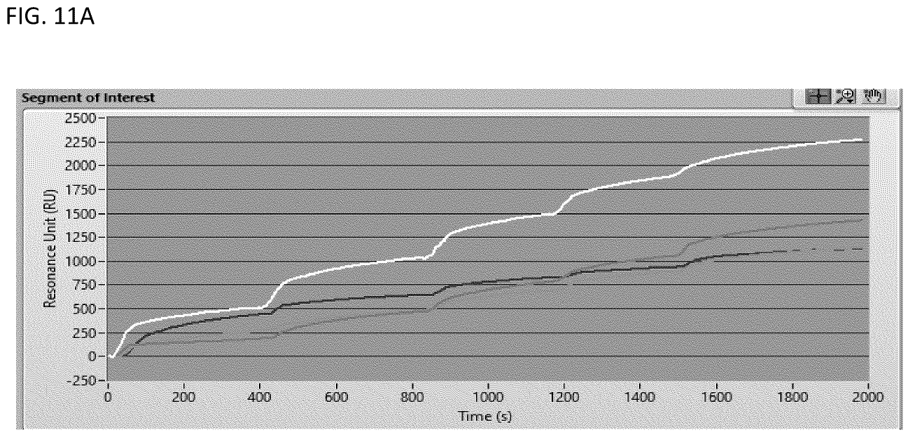

The immobilization of the gold-binding fusion protein which is Protein G (SEQ ID NO:19 with a linker [SEQ ID NO: 2] fused to gold binding protein SEQ ID NO: 4 (fusion known as “EMT-003”) and a reference sample onto gold sensor chips using direct immobilization (FIG. 8) and EDC-NHS conjugation (FIG. 9) techniques were evaluated by SPR (portable, 4-channel P4SPR device, Affinite Instruments).

As shown in Table 5 below, the gold-binding fusion protein showed a three-fold increase in Resonance Units (RU) during the immobilization phase by direct binding (2300 RU) compared to EDC-NHS process (750 RU). These results show that direct immobilization on gold is significantly more efficient than the immobilization using the EDC-NHS Process.

TABLE 5
Immobilization of Gold-binding Fusion Protein Direct
Binding Versus EDC-NHS Conjugation Using SPR.

| Immobilization Method | Ligand | Rmax (RU) |
|---|---|---|
| Direct Binding | Gold fusion protein EMT-003 | 2300 |
| EDC-NHS conjugation | Gold fusion protein EMT-003 | 750 |

### EXAMPLE 10

Evaluating the Sensitivity and Limit of Detection (LoD) for the Binding of SARS-CoV-2 Spike Protein Antigen and SARS-CoV-2 Nucleocapsid Protein Antigen to SARS-CoV-2 Spike and Nucleocapsid Antibodies Conjugated to Gold-Binding Fusion Protein on Gold Sensors Prepared by Direct Immobilization or EDC-NHS Conjugation:

First, gold sensors immobilized with gold-fusion protein by direct binding or EDC-NHS techniques according to Example 9, were conjugated with SARS-CoV-2 Spike or Nucleocapsid antibodies, and then SARS-CoV-2 Spike antigen or the negative control (SARS-CoV-2 Nucleocapsid antigen) following the method outlined in Example 3.

Surface plasmon resonance (SPR) was used to evaluate the sensitivity and LoD for the binding of SARS-CoV-2 Spike protein antigen and SARS-CoV-2 Nucleocapsid protein antigen to SARS-CoV-2 Spike or nucleocapsid antibodies conjugated to gold-fusion protein, which was immobilized on gold sensors by direct binding or EDC-NHS immobilization techniques from Example 9. As shown in FIG. 10, the EDC-NHS immobilization technique (left panel) has an impact on the sensitivity and LoD. Approximately a two-fold increase in sensitivity was observed for the Spike antigen (Indicated by S in FIG. 10) and approximately a 1.3-fold increase in sensitivity for the Nucleocapsid antigen (Indicated by NC in FIG. 10). These results show that the gold-binding fusion protein EMT003 immobilized by direct binding onto gold sensors was better than traditional SPR, and especially noticeable for Spike protein detection.

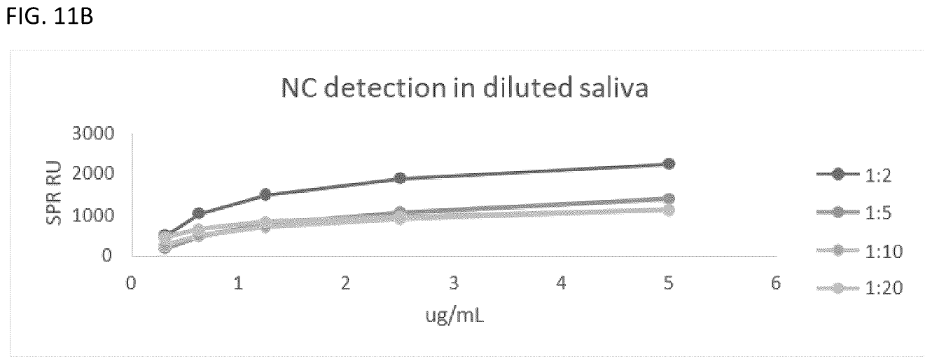

The detection of nucleocapsid antigen using the direct binding EMT-003 gold fusion protein-based SPR system in saliva (human, pooled) was then evaluated. As shown in FIGS. 11A and 11B, recombinant nucleocapsid antigen binding was visible at all dilution. Detection was highest at 1:2 saliva in Running Buffer.

### EXAMPLE 11

SARS-CoV-2 Spike Protein Detection by SPR: This example evaluated the performance of EMT003 coupled to an antibody for the selective detection of antigens under SPR. Specifically, EMT003 coupled to a SARS-CoV-2 anti-spike protein antibody was evaluated for the selective detection of spike protein. EMT003 was diluted to 10 μg/mL. Next, a clean gold coated sensor for SPR was loaded into the flow modules in the instrument. 500 μL of distilled water and 500 μL of PBS were flowed over the sensors briefly to establish the baseline signal. The fusion protein EMT003 was then flowed over the sensor for 10 minutes. Then 500 μL of PBS was flowed over the gold surface to removed poorly adsorbed EMT003 fusion protein. All measurements were performed at room temperature.

Two different types of antibodies were coupled to EMT003 over multiple SPR channels. First, 10 μg/mL of an anti-spike antibody was flowed over in channels B, C and D. As a negative control, 10 μg/mL of anti-TGFB was injected in channel A. Then, two wash steps with PBS and PBST were performed to remove excess of poorly absorbed antibody to EMT003. Finally, a blocking step with BSA was included to prevent potential non-specific binding to the sensor surface of spike protein during the titration step.

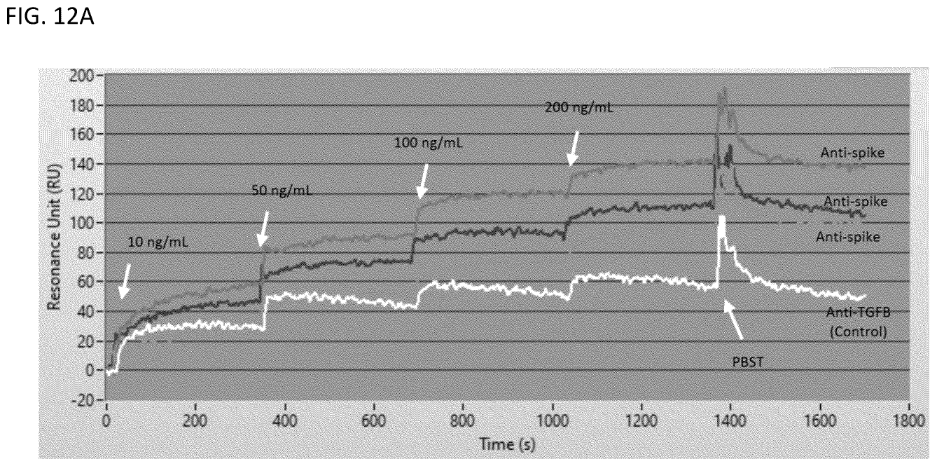

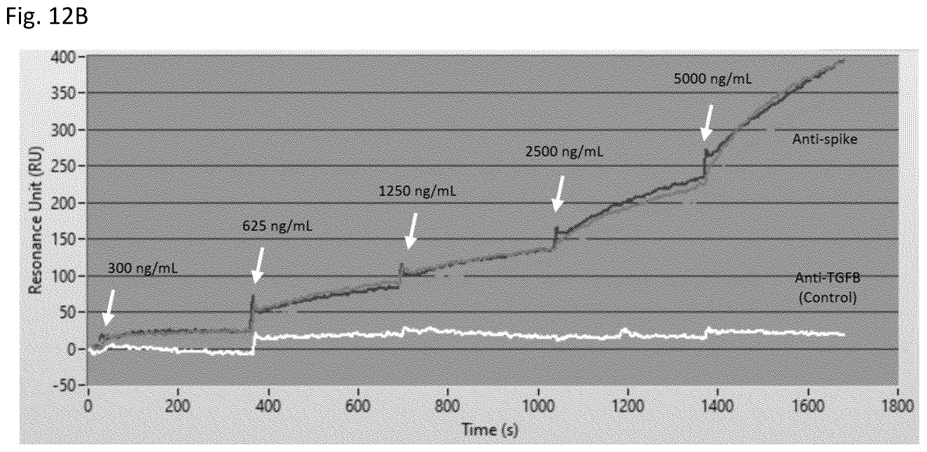

The titration with clinically relevant concentrations of SARS-CoV-2 spike protein consisted of four injections at gradually increasing concentration of: 10, 50, 100 and 200 ng/mL. The SPR real time bind profile is provided in FIG. 12A. The shift in RU is more evident at concentrations above 100 ng/mL of spike protein for SARS-CoV-2 anti-spike antibody (red, blue and green lines) than for anti-TGFB antibody.

An additional titration with high concentrations of SARS-CoV-2 spike protein consisted of five injections at gradually increasing concentration of: 300, 625, 1250, 2500 and 5000 ng/mL was also performed. This is indicated in FIG. 12B. The Shift in RU is significantly different between SARS-CoV-2 anti-spike antibody and negative control for anti-TGFB antibody.

Conclusion: EMT003 coupled with anti-spike antibody was able to detect as low as 100 ng/mL of recombinant spike antigen. EMT003 coupled with anti-spike antibody can detect higher concentrations of recombinant spike protein in a linear and specific manner. The test is also specific, as EMT003 coupled with anti-TGFB did not detect spike protein as expected for the negative control.

### EXAMPLE 12

Generation and Purification of Streptavidin Fusion Proteins in Escherichia coli:

The protein sequences for the fusion proteins, containing gold-binding peptides from Table 6 below, fused to a linker and streptavidin were converted to Streptavidin fusion proteins in an E. coli pET-30a (+) expression vector using the same cloning and purification strategy described in Example 1.

TABLE 6
Fusion Sequences

| ID | Target | Fusion Sequence |
|---|---|---|
| EMT027 | Gold | SEQ ID NO: 22-SEQ ID NO: 1- |
| | | SEQ ID NO: 4 |
| EMT028 | Gold | SEQ ID NO: 22-SEQ ID NO: 1- |
| | | SEQ ID NO: 8 |

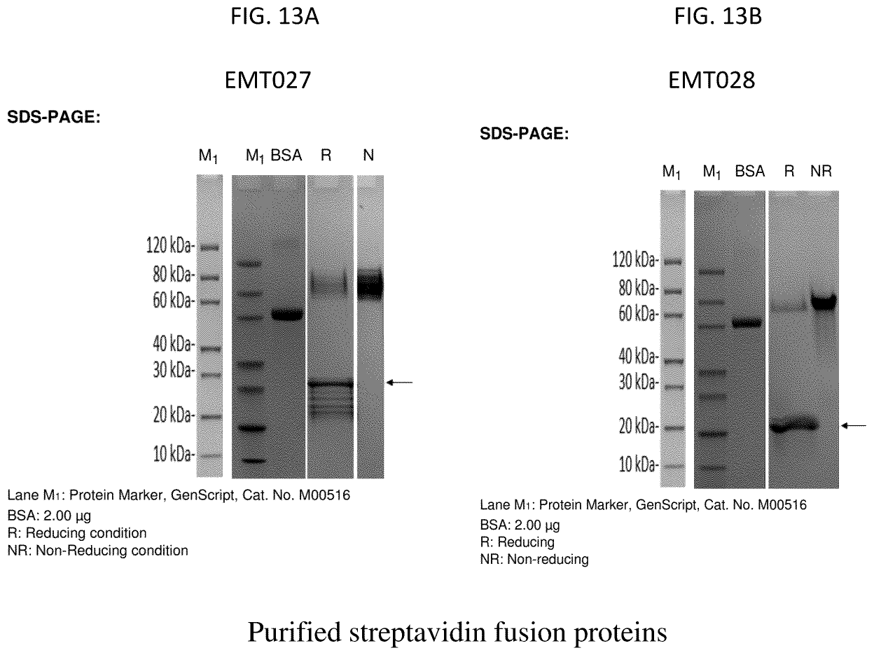

As shown in FIG. 13 gels A), and B) show the expression and purity of the gold-binding streptavidin fusion proteins on Coomassie-stained SDS-PAGE gels. 2 μg of BSA was added in lane 1 of each gel A), and B). Gel A) shows full Gold-binding streptavidin fusion protein EMT027, and Gel B) shows full Gold-binding streptavidin fusion protein EMT028.

### EXAMPLE 13

Lateral flow assay application of streptavidin fusion proteins: Gold-binding streptavidin fusion proteins EMT027 and EMT028 were conjugated to gold nanoparticles according to the method outlined in Example 5. Both gold binding streptavidin fusion proteins bound successfully to gold nanoparticles across a range of pH.

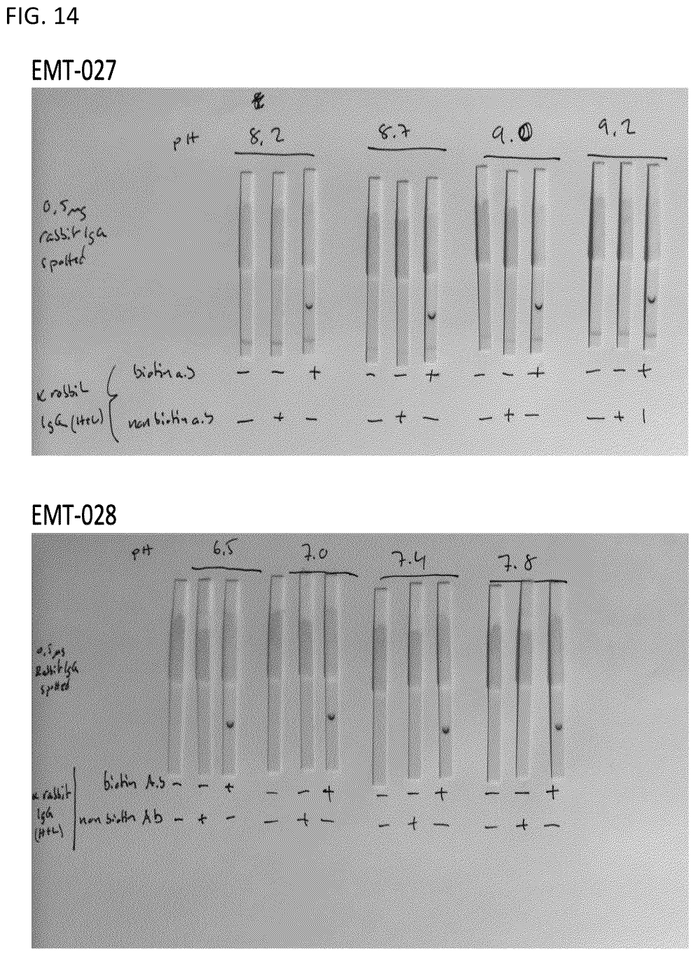

Immobilization of biotinylated detection antibodies onto gold-binding streptavidin fusion protein coated gold nanoparticles and their antigen binding capacity was then tested using a lateral flow assay. In this assay, the antigen (rabbit IgG antibody) was directly dotted on the strip membrane. Biotinylated detection antibody (anti-rabbit IgG) was loaded onto streptavidin fusion proteins (EMT027 and EMT028) immobilized on gold nanoparticles, and then allowed to flow up the membrane. As shown in FIG. 14, immobilized antigen on the strips can be detected by both EMT027 and EMT028-based conjugates (i.e. gold nanoparticle-streptavidin fusion protein-biotin conjugated detection antibody complex) in a lateral flow assay. Specifically, 0.5 μg of rabbit antigen (rabbit IgG antibody) was spotted on to the membrane. Then, biotinylated anti-rabbit IgG or non-biotinylated anti-rabbit IgG was loaded with either streptavidin fusion proteins (EMT027 and EMT028) immobilized on gold nanoparticles at various pH for each fusion. FIG. 14 shows three strips at each pH wherein there was no anti-rabbit IgG at all was loaded (left strip), a biotinylated anti-rabbit IgG with streptavidin fusion (middle strip) and a non-biotinylated anti-rabbit IgG with streptavidin fusion (right strip), indicating specificity of the anti-rabbit IgG specifically bonding to the antigen when loaded and bound to the fusion protein (EMT027 or EMT028).

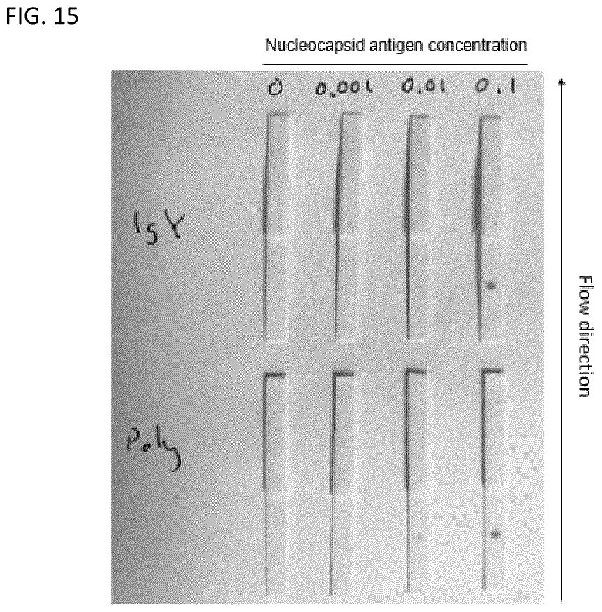

The nucleocapsid antigen binding capacity of the EMT028-based gold nanoparticle conjugate was then tested in a ‘dotted’ sandwich lateral flow assay. In this assay, polyclonal anti-nucleocapsid antigen capture antibodies (chicken, top and rabbit, bottom) were dotted on the membrane. The EMT028-based gold nanoparticle conjugate was then mixed with nucleocapsid antigen and allowed to flow up the membrane. As shown in FIG. 15, the EMT028-based gold nanoparticle conjugate loaded with biotin-detection antibody (anti-nucleocapsid) successfully detected nucleocapsid antigen in the dotted sandwich lateral flow assay. Two different capture antibodies were evaluated and showed comparable results.

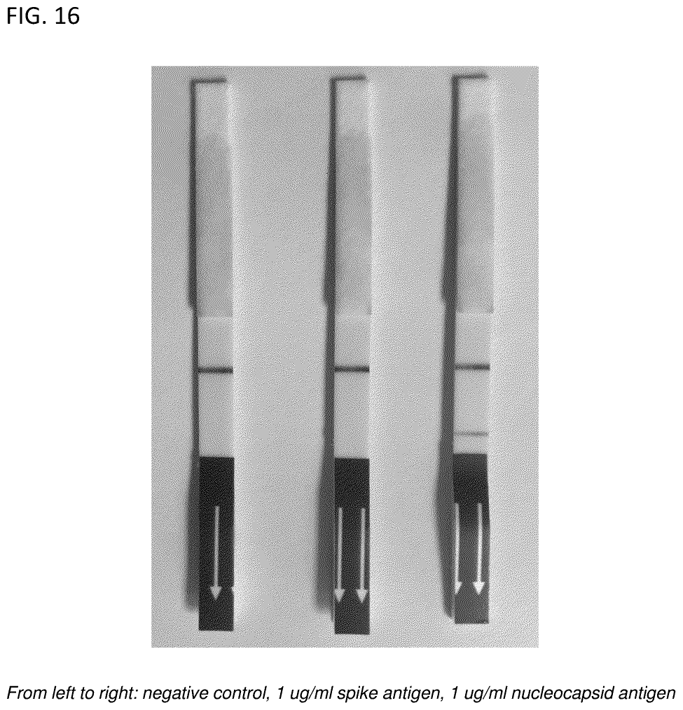

The specificity of the EMT028-based gold nanoparticle conjugate system for nucleocapsid antigen was tested in a striped sandwich lateral flow assay. As shown in FIG. 16, the EMT028-based conjugate coupled to nucleocapsid antibody successfully detected the nucleocapsid antigen but not spike antigen in the striped sandwich lateral flow assay. No non-specific binding was observed to the spike protein at 1 ug/ml while a clear signal was obtained for the sample with nucleocapsid antigen. No non-specific binding was observed in the negative control sample. Together these results show the specificity of the assay.

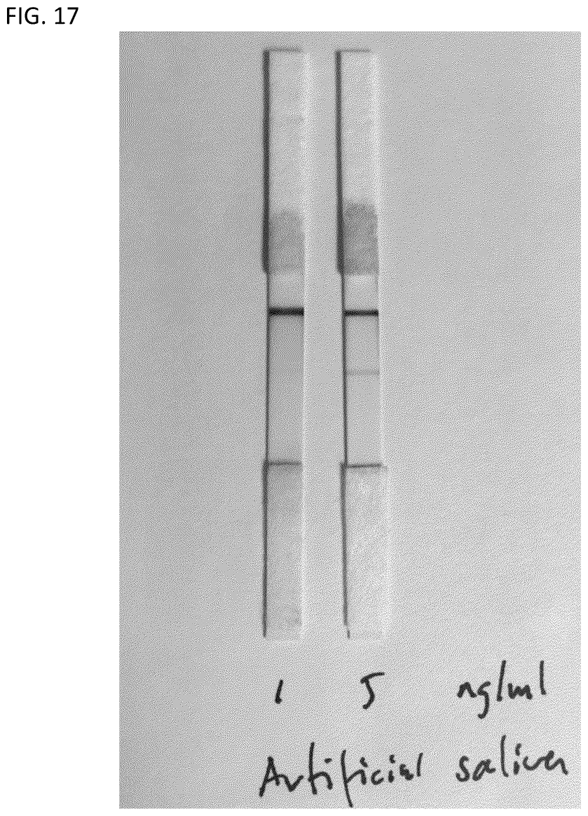

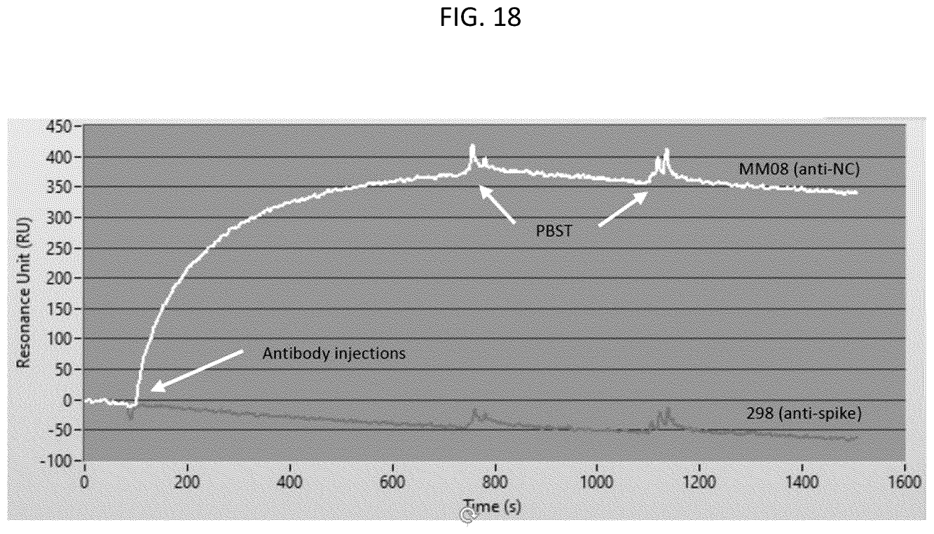

The detection of nucleocapsid antigen at 1 ng/ml and 5 ng/ml in artificial saliva with mucin by EMT028 conjugate was also evaluated. In this assay, a sample volume of 60 μL was applied to each lateral flow strip. As shown in FIG. 17, at the time of assay completion, a band was clearly visible in both samples. These results show EMT028 conjugate coupled to nucleocapsid antibody in striped sandwich lateral flow assay successfully detects the nucleocapsid antigen in artificial saliva.

### EXAMPLE 14

Screening of Nucleocapsid Antibody Using EMT028/Biotin-Nucleocapsid on SPR:

This study was performed to evaluate streptavidin fusion protein EMT028 coupled with SARS-CoV-2 biotinylated nucleocapsid protein for antibody detection as the analyte using SPR.

First, EMT028 was diluted to 10 μg/mL. Next, a clean gold coated sensor was loaded into the flow modules in the SPR instrument. 500 μL of distilled water and 500 μL of PBS were flowed over the sensors briefly to establish the baseline signal. The fusion protein EMT028 was then flowed over the sensor for 10 minutes. Then PBS and PBS-Tween (0.005%) was flowed over the gold surface to remove poorly adsorbed EMT028 fusion protein.

As a second layer in the system, a biotinylated nucleocapsid protein was coupled to EMT028. Then, one wash step with PBST was performed to remove excess biotinylated protein. Finally, a blocking step with 1% BSA was included to prevent potential non-specific binding. 10 μl/mL of anti-nucleocapsid antibody MM08 was flowed in channel A, whereas an anti-spike antibody was injected in channel B (as a negative control). See FIG. 18.

The interaction between anti-nucleocapsid MM08 antibody and biotinylated nucleocapsid protein showed a significant increase in the signal shift. This signal remained constant even after two PBST rinses suggesting a strong and stable binding. No shift in signal was observed when anti-spike was flowed over EMT028/biotin-nucleocapsid no major signal shift was observed for the interaction between anti-spike 298 and biotinylated nucleocapsid protein.

Conclusion: EMT028 coupled with biotinylated nucleocapsid protein was able to detect anti-nucleocapsid MM08 antibody at a concentration of 10 μg/mL, with no detection of binding to a non-nucleocapsid antibody, indicating a detection system that is both sensitive and specific.

### EXAMPLE 15

Generation and purification of gold-binding and bispecific Immunoglobulin A and Bispecific Antibody fragments. Bispecific antibodies and antibody fusion fragments are made as known in the art. Specifically, the genes of different antibodies or antibody fragments are cloned and transfected into Expi-CHO cells (Thermofisher), then were purified by AKTA Explorer protein purification system.

A bispecific immunoglobulin A dimer is cloned, expressed and purified wherein one antibody monomer has high affinity for gold and the other antibody monomer of the fused immunoglobulin A dimer has a high affinity for SARS-CoV-2 Spike protein.

Surface plasmon resonance (SPR), an opto-electronic biosensing technique, is chosen to evaluate the binding kinetics of the bispecific immunoglobulin A fusion to a gold surface. First, the immobilization of the bispecific immunoglobulin A fusion and two controls (ISBP-free fusion protein and buffer only) is evaluated. Zero or minimal binding is observed for those controls. The bispecific immunoglobulin A fusion, however, shows a ten-fold increase in Resonance Units (RU) during the immobilization phase compared to the ISBP-free control version. After it is established that bispecific immunoglobulin A fusion is bound to the gold surface, a dilution series for the Spike antigen is performed spanning the following concentrations in two experiments: 1.5625 nM (×2), 3.125 nM, 6.25 nM, 12.5 nM, 25 nM, 50 nM and 100 nM. The raw data is analyzed using Biacore™ 8K Evaluation software version 1.1. It is shown that SARS-CoV-2 Spike antibody binds to the bispecific immunoglobulin A fusion with a KD of between 1 to 2 E-10 M.

### EXAMPLE 16

In another example, a bispecific antibody fragment fusion with a gold binding VH domain and a scFv specific to SARS-CoV-2 Spike protein is cloned, expressed and purified using various methods known in the art. In a specific example, the fusions will be cloned into a phagemid or other known cloning vector. The fusions, which comprise a 6×His-tag, and are to be cloned into an expression vector and transformed in the BL21 (DE3) competent cell line and expression system. The transformation is performed under such a condition that heat shock is performed in ice→42° C.×90 sec→in ice. 750 μL of LB medium is added to the BL21 solution transformed by heat shock, and the whole was cultured with shaking for 1 hour at 37° C. After that, centrifugation is performed at 6,000 rpm×5 min, and 650 μL of the culture supernatant is discarded. The remaining culture supernatant and a cell fraction as a precipitate is stirred and inoculated on an LB/amp. plate, and the whole is left standing at 37° C. overnight.

Main Culture and Expression

Once a clone is confirmed to have the intended fusion protein, a preculture solution with the clone is subcultured in 750 mL of a 2×YT medium, and the culture is further continued at 28° C. When OD₆₀₀ exceeded 0.8, IPTG is added to have a final concentration of 1 mM, and culture is performed at 28° C. overnight.

Purification

The fusion protein is purified from an insoluble granule fraction through the following steps:

(i) Collection of Insoluble Granule

The culture solution is centrifuged at 6,000 rpm×30 min to obtain a precipitate as a bacterial fraction. The resultant is suspended in a Tris solution (20 mM Tris/500 mM NaCl) in ice. The resultant suspension is then homogenized with a French press to obtain a homogenized solution. Next, the homogenized solution is centrifuged at 12,000 rpm×15 min, and the supernatant is removed to obtain a precipitate as an insoluble granule fraction comprising the inclusion bodies.

The insoluble fraction is then immersed overnight in 10 mL of a 6 M guanidine hydrochloride/Tris solution. Next, the resultant is centrifuged at 12,000 rpm×10 min to obtain a supernatant as a solubilized solution.

(ii) Metal Chelate Column

A Ni column is used as a metal chelate column carrier. Column adjustment, sample loading, and a washing step are performed at room temperature (20° C.). Elution of a His tag-fused fusion protein as a target is performed in a 60 mM imidazole/Tris solution.

(iii) Refolding

The sample comprising the fusion proteins is refolded using dialysis and is immersed in a 6 M guanidine hydrochloride/Tris solution and dialyzed for 6 hours while being gently stirred. The concentration of the guanidine hydrochloride solution of the external solution is slowly reduced over time in a stepwise manner into a PBS buffer wherein the fusion with a gold binding VH domain and a scFv specific to SARS-CoV-2 Spike protein is refolded appropriately. Surface plasmon resonance (SPR), an opto-electronic biosensing technique, is chosen to evaluate the binding kinetics of a bispecific antibody fragment to a gold surface. First, the immobilization of the bispecific antibody fragment fusion and two controls (ISBP-free fusion protein and buffer only) is evaluated. Zero or minimal binding is observed for those controls. The bispecific antibody fragment fusion, however, shows a ten-fold increase in Resonance Units (RU) during the immobilization phase compared to the ISBP-free control version. After it is established that bispecific antibody fragment fusion is bound to the gold surface, a dilution series for the Spike antigen is performed spanning the following concentrations in two experiments: 1.5625 nM (×2), 3.125 nM, 6.25 nM, 12.5 nM, 25 nM, 50 nM and 100 nM. The raw data is analyzed using Biacore™ 8K Evaluation software version 1.1. It is shown that SARS-CoV-2 Spike antibody binds to the bispecific antibody fragment fusion with a KD of between 1 to 2 E-10 M.

### EXAMPLE 17

Binding Analysis of Synthetic Binding Proteins to Silica, Polystyrene, and Cellulose Fused to Streptavidin and Sensors Using QCM-D:

The protein sequences for the fusion proteins, containing cellulose, polystyrene or silica binding peptides from Table 7 below, fused to a linker and streptavidin were converted to fusion proteins in an E. coli pET-30a (+) expression vector using the same cloning and purification strategy described in Example 1.

TABLE 7
Surface Target Proteins Streptavidin Fusion Proteins

| ID | Target | Fusion Sequence |
|---|---|---|
| EMT029 | Silica | SEQ ID NO: 22-SEQ ID NO: 1- |
| | | SEQ ID NO: 25 |
| EMT032 | Cellulose | SEQ ID NO: 22-SEQ ID NO: 1- |
| | | SEQ ID NO: 15 |
| EMT033 | Cellulose | SEQ ID NO: 22-SEQ ID NO: 1- |
| | | SEQ ID NO: 16 |
| GL008 | Polystyrene | SEQ ID NO: 22-SEQ ID NO: 1- |
| | | SEQ ID NO: 17 |
| GL009 | Polystyrene | SEQ ID NO: 22-SEQ ID NO: 1- |
| | | SEQ ID NO: 18 |

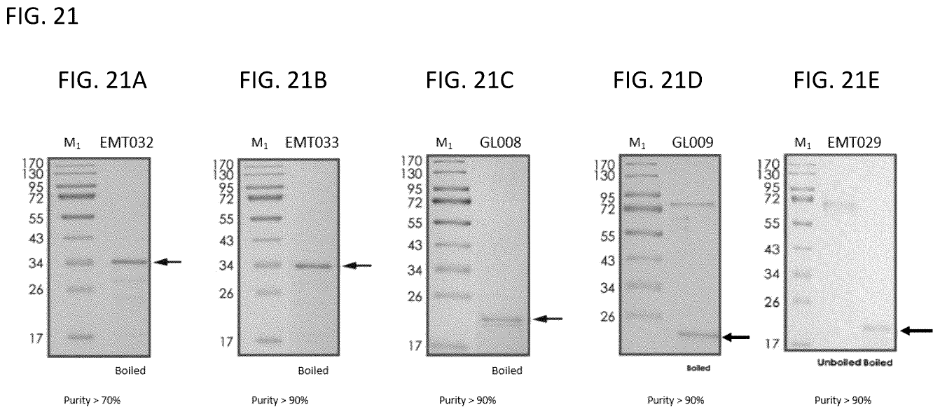

FIG. 21 shows Coomassie-stained SDS-PAGE gels indicating the expression and purity of cellulose-binding streptavidin fusion proteins (FIGS. 21A-B), and polystyrene-binding streptavidin fusion proteins (FIG. 21C-D), silica-binding streptavidin fusion proteins (FIG. 21 E)) of the specific fusion proteins described in Table 7.

For analyte detection, the Table 7 fusion proteins were loaded on the respective silica, polystyrene, or cellulose sensors as the target surface as indicated in Table 7, using quartz crystal microbalance with dissipation monitoring (QCM-D). All Table 7 fusion proteins were diluted in 1×PBS solution in Type 1 water to a concentration of 25 μg/ml.

BSA was diluted to 100 μg/mL using the same PBS solution. All biotinylated antibodies for binding to the streptavidin and the respective antigens for detection were diluted in 1×PBS solution in Type 1 water to a concentration of 25 μg/ml. This includes Troponin (antigen), anti-Troponin antibody, and biotinylated troponin antibody.

Each QCM sensor was primed with PBS for about 3 hrs; each sensor was then washed with new PBS for 5 min. Each fusion peptide diluted in PBS solution was loaded on the respective sensor with the indicated inorganic surface for 1 hr. After absorption of the fusion peptide to the surface, the sensor was washed with 30 min of PBS, followed by 30 min of BSA solution, followed by 30 min of PBS. A biotinylated troponin antibody was then loaded onto the surface for 40 min, followed by 30 min of PBS. Troponin antigen was then added for 15 min, followed by another 30 min wash of PBS.

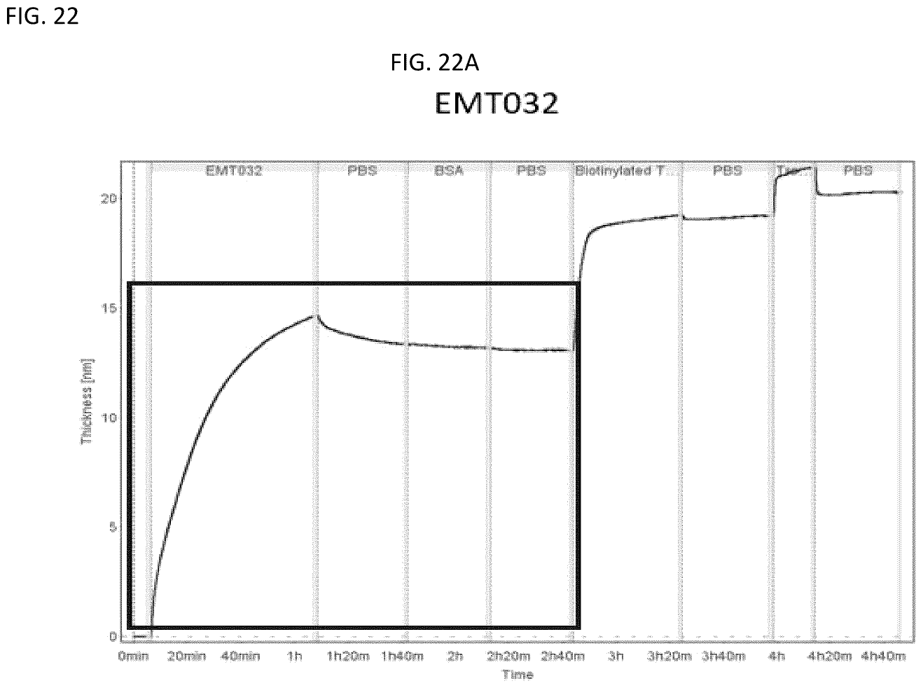

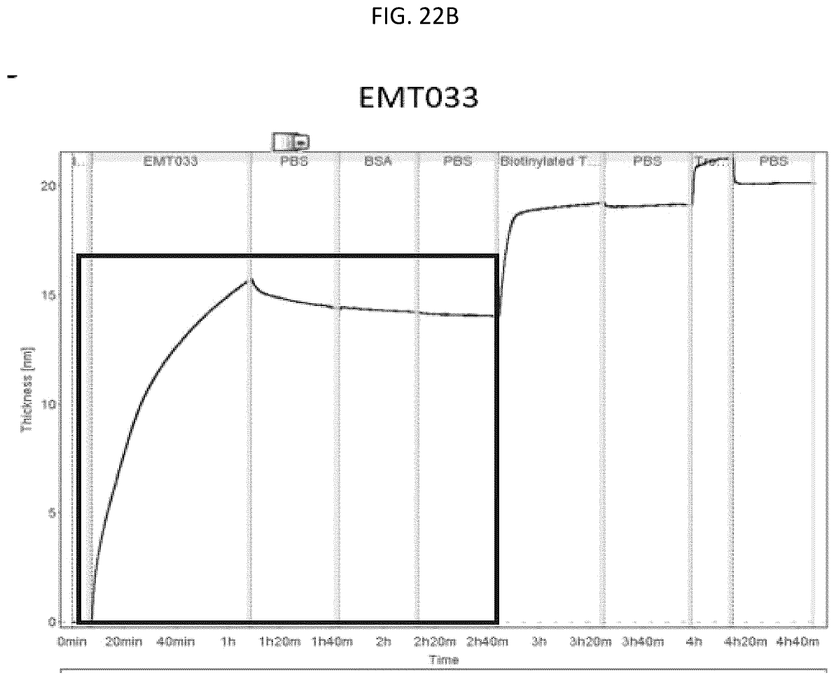

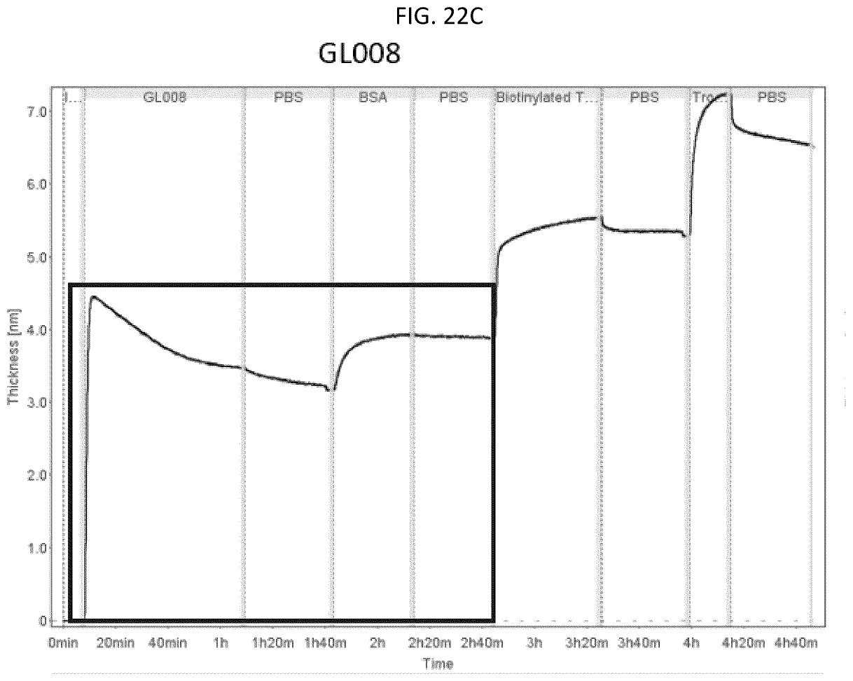

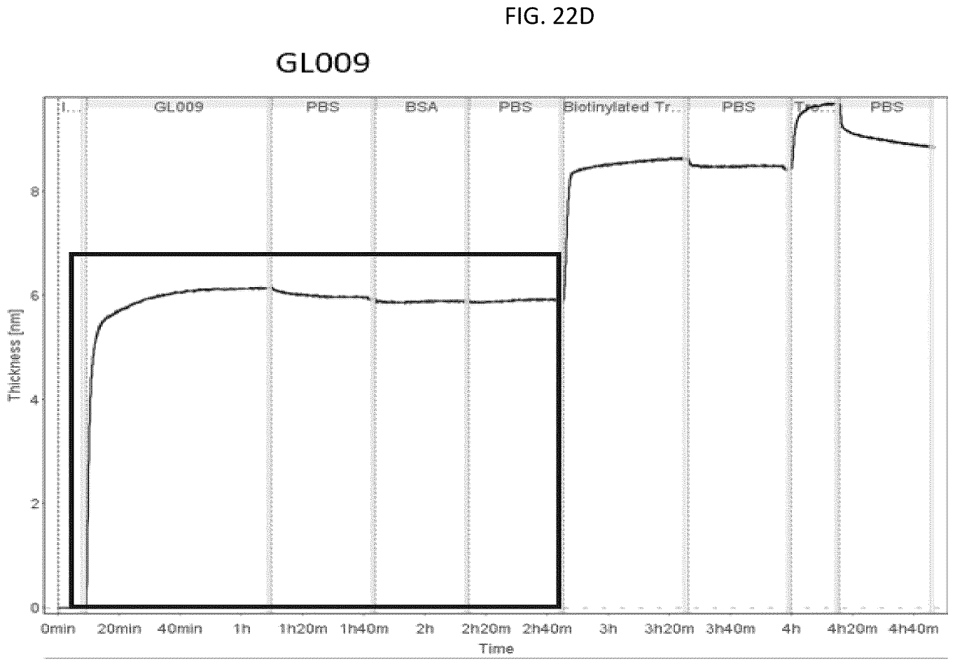

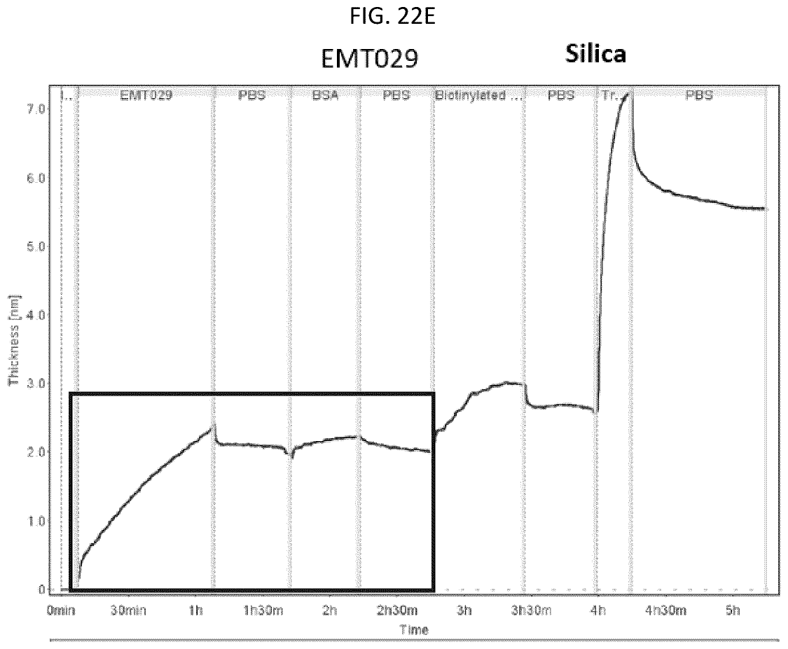

Table 8 and 9 below summarizes the modeled mass and thickness values for each step of these QCM sensor experiments. The sensorgrams are indicated in FIG. 22A-E.

TABLE 8
Modeled Sauerbrey Mass:

| | Sensor 4: | Sensor 5: | Sensor 6: | Sensor 7: | Sensor 8; |
|---|---|---|---|---|---|
| | EMT029 | EMT032 | EMT033 | GL008 | GL009 |
| | Sauer- | Sauer- | Sauer- | Sauer- | Sauer- |
| | Mass | Mass | Mass | Mass | Mass |
| Step | (ng/cm²) | (ng/cm²) | (ng/cm²) | (ng/cm²) | (ng/cm²) |
| Fusion | 252.2 | 1758.4 | 1879.7 | 417.0 | 737.8 |
| Protein | | | | | |
| PBS | 221.5 | 1604.7 | 1730.8 | 381.3 | 713.0 |
| BSA | 241.8 | 1583.0 | 1705.6 | 471.7 | 707.2 |
| PBS | 218.9 | 1568.9 | 1684.6 | 466.0 | 710.4 |
| Biotinylated | 298.7 | 2311.8 | 2307.7 | 665.1 | 1037.2 |
| Troponin | | | | | |
| Antibody | | | | | |
| PBS | 269.7 | 2304.9 | 2298.1 | 635.4 | 1013.2 |
| Troponin | 760.8 | 2572.9 | 2555.3 | 869.2 | 1164.3 |
| Antigen | | | | | |
| PBS | 614.4 | 2437.4 | 2417.5 | 784.9 | 1064.9 |

TABLE 9
Modeled Sauerbrey Thickness:

| | Sensor 4: | Sensor 5: | Sensor 6: | Sensor 7: | Sensor 8: |
|---|---|---|---|---|---|
| | EMT029 | EMT032 | EMT033 | GL008 | GL009 |
| | Sauer- | Sauer- | Sauer- | Sauer- | Sauer- |
| | Thickness | Thickness | Thickness | Thickness | Thickness |
| Step | (nm) | (nm) | (nm) | (nm) | (nm) |
| Fusion | 2.1 | 14.7 | 15.7 | 3.5 | 6.1 |
| Protein | | | | | |
| PBS | 1.8 | 13.4 | 14.4 | 3.2 | 5.9 |
| BSA | 2.0 | 13.2 | 14.2 | 3.9 | 5.9 |
| PBS | 1.8 | 13.1 | 14.0 | 3.9 | 5.9 |
| Biotinylated | 2.5 | 19.3 | 19.2 | 5.5 | 8.6 |
| Troponin | | | | | |
| Antibody | | | | | |
| PBS | 2.2 | 19.2 | 19.2 | 5.3 | 8.4 |
| Troponin | 6.3 | 21.4 | 21.3 | 7.2 | 9.7 |
| Antigen | | | | | |
| PBS | 5.1 | 20.3 | 20.1 | 6.5 | 8.9 |

FIG. 22A-E shows the absorption changes for all Table 7 fusions under this protocol. Specifically, FIGS. 22 A and B shows the absorption by detecting nanometer thickness of GL008 and GL009 on a polystyrene surface respectively. Notably, GL008 and GL009 Polystyrene-binding fusion proteins showed different adsorptions: GL009 showed a final adsorption of 5.9 nm after PBS rinse, versus 3.2 nm for GL008. Initially, GL008 adsorption was similar to GL009 for at least 5 nm of protein adsorption, before sudden desorption during the adsorption protein step.

Regardless, after fusion protein binding, there was minimal absorption of BSA blocking agent, but substantial absorption of the biotinylated troponin antibody indicating selective binding to streptavidin. Detection of binding to the intended antigen (troponin) is also detected in both.

FIGS. 22 C and D shows the absorption by detecting nanometer thickness of EMT032 and EMT033 on a cellulose surface respectively. Both cellulose-binding fusion proteins were found to bind to the cellulose sensor surface. Only a very small fraction washed off during the subsequent wash step. No significant changes to the thickness or mass of the layers occurred during the subsequent blocking with BSA and washing steps. There was substantial absorption of the biotinylated troponin antibody indicating selective binding to streptavidin. These figures, however, show that the Troponin Antigen was minimally adsorbed relative to other surfaces or fusion peptides.

FIG. 22 E shows the absorption by detecting nanometer thickness of EMT029 on a silica surface respectively. Despite significantly less fusion protein adsorption compared with the other sensors, further EMT029 fusion protein adsorption was likely if flow-times were extended beyond 1 hour for this step, based on the slope of the raw data frequency observed (i.e., this step had not approached full equilibrium yet). There was also indication of absorption of the biotinylated troponin antibody indicating selective binding to streptavidin, particularly when compared to the PBS blocker. Lastly, there was substantial absorption when Troponin Antigen was added.

### EXAMPLE 18

Bispecific scFv Antibodies:

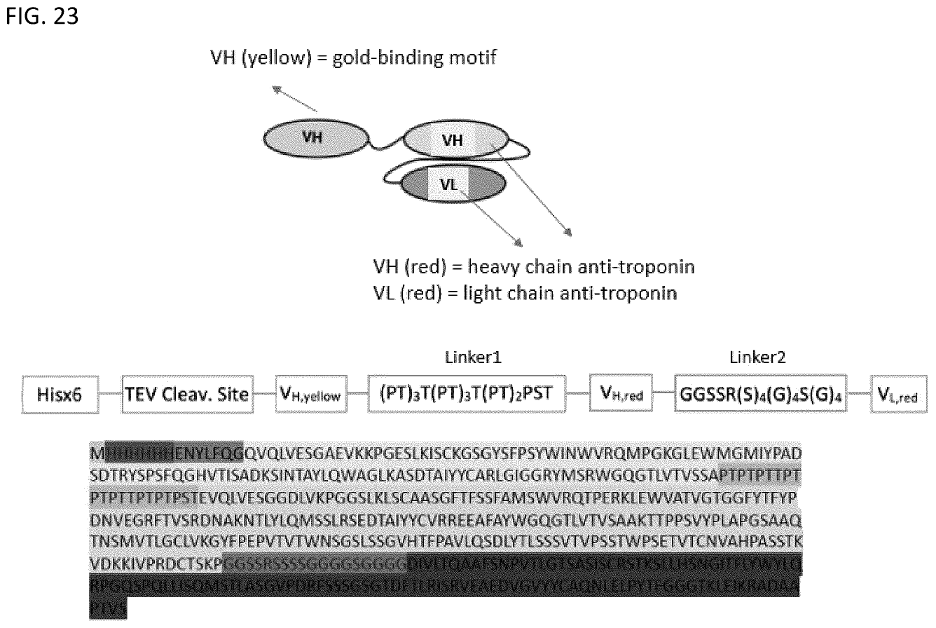

In another example, a bispecific antibody fragment fusion with a gold binding VH domain and a scFv specific to troponin was cloned, expressed and purified using various methods known in the art. The scFv Troponin fusion (GL007) includes the sequence below in Table 10 and as diagramed in FIG. 23.

TABLE 10
scFv Antibody-Linker Sequences

| | | | | Molecular | | |
|---|---|---|---|---|---|---|
| | | | Length | weight | | SEQ ID |
| ID | Target | Peptide Sequence | (aa) | (Dalton) | Reference | NO: |
| GL007 | Fusion of gold | MHHHHHHENYLFQGQVQ | 518 | | | SEQ ID |
| | binding VH | LVESGAEVKKPGESLKISC | aa | | | NO: 29 |
| | domain to | KGSGYSFPSYWINWVRQ | | | | |
| | bispecific | MPGKGLEWMGMIYPADS | | | | |
| | scFv Antibody | DTRYSPSFQGHVTISADKS | | | | |
| | to troponin | INTAYLQWAGLKASDTAI | | | | |
| | | YYCARLGIGGRYMSRWG | | | | |
| | | QGTLVTVSSAPTPTPTTPT | | | | |
| | | PTPTTPTPTPSTEVOLVES | | | | |
| | | GGDLVKPGGSLKLSCAAS | | | | |
| | | GFTFSSFAMSWVRQTPER | | | | |
| | | KLEWVATVGTGGFYTFYP | | | | |
| | | DNVEGRFTVSRDNAKNTL | | | | |
| | | YLQMSSLRSEDTAIYYCV | | | | |
| | | RREEAFAYWGQGTLVTV | | | | |
| | | SAAKTTPPSVYPLAPGSA | | | | |
| | | AQTNSMVTLGCLVKGYF | | | | |
| | | PEPVTVTWNSGSLSSGVH | | | | |
| | | TFPAVLQSDLYTLSSSVTV | | | | |
| | | PSSTWPSETVTCNVAHPA | | | | |
| | | SSTKVDKKIVPRDCTSKP | | | | |
| | | GGGGSGGGGSGGGGSAS | | | | |
| | | GGGDIVLTQAAFSNPVTL | | | | |
| | | GTSASISCRSTKSLLHSNGI | | | | |
| | | TFLYWYLQRPGQSPQLLIS | | | | |
| | | QMSTLASGVPDRESSSGS | | | | |
| | | GTDFTLRISRVEAEDVGV | | | | |
| | | YYCAQNLELPYTFGGGTK | | | | |
| | | LEIKRADAAPTVS | | | | |
| | VH Anti- | EVQLVESGGD | 222 | | https:// | SEQ ID |
| | troponin | LVKPGGSLKL | aa | | www.ncbi. | NO: 32 |
| | domain | SCAASGFTFS | | | nlm.nih. | |
| | | SFAMSWVRQT | | | gov/ | |
| | | PERKLEWVAT | | | protein/ | |
| | | VGTGGFYTFY | | | AAR83243.1 | |
| | | PDNVEGRFTV | | | | |
| | | SRDNAKNTLY | | | | |
| | | LQMSSLRSED | | | | |
| | | TAIYYCVRRE | | | | |
| | | EAFAYWGQGT | | | | |
| | | LVTVSAAKTT | | | | |
| | | PPSVYPLAPG | | | | |
| | | SAAQTNSMVT | | | | |
| | | LGCLVKGYFP | | | | |
| | | EPVTVTWNSG | | | | |
| | | SLSSGVHTFP | | | | |
| | | AVLQSDLYTL | | | | |
| | | SSSVTVPSST | | | | |
| | | WPSETVTCNV | | | | |
| | | AHPASSTKVD | | | | |
| | | KKIVPRDCTS | | | | |
| | | KP | | | | |
| | VLAnti- | DIVLTQAAFS | 121 | | https:// | SEQ ID |
| | troponin | NPVTLGTSAS | aa | | www.ncbi. | NO: 33 |
| | domain | ISCRSTKSLL | | | nlm.nih. | |
| | | HSNGITFLYW | | | gov/ | |
| | | YLQRPGQSPQ | | | protein/ | |
| | | LLISQMSTLA | | | AAR83244.1 | |
| | | SGVPDRFSSS | | | | |
| | | GSGTDFTLRI | | | | |
| | | SRVEAEDVGV | | | | |
| | | YYCAQNLELP | | | | |
| | | YTFGGGTKLE | | | | |
| | | IKRADAAPTV | | | | |
| | | S | | | | |

Specifically, FIG. 23 shows a 6×His-tag fused to a TEV Cleavage site, followed by a VH-domain that is a gold binding motif, followed by Linker 1, then followed by VH Anti-troponin domain, followed by a Linker, then followed by a VL Anti-troponin domain.

This fusion was cloned into an expression vector and expression system well known in the art. The fusion protein is purified from an insoluble granule fraction through the following steps:

(i) Collection of Insoluble Granule

The culture solution was centrifuged at 6,000 rpm×30 min to obtain a precipitate as a bacterial fraction. The resultant was suspended in a Tris solution (20 mM Tris/500 mM NaCl) in ice. The resultant suspension was then homogenized with a French press to obtain a homogenized solution. Next, the homogenized solution was centrifuged at 12,000 rpm×15 min, and the supernatant was removed to obtain a precipitate as an insoluble granule fraction comprising the inclusion bodies.

The insoluble fraction was then immersed overnight in 10 mL of a 6 M guanidine hydrochloride/Tris solution. Next, the resultant was centrifuged at 12,000 rpm×10 min to obtain a supernatant as a solubilized solution.

(ii) Metal Chelate Column

A Ni column was used as a metal chelate column carrier. Column adjustment, sample loading, and a washing step was performed at room temperature (20° C.). Elution of a His tag-fused fusion protein as a target was performed in a 60 mM imidazole/Tris solution.

(iii) Refolding

The sample comprising the fusion proteins was refolded using dialysis and was immersed in a 6 M guanidine hydrochloride/Tris solution and dialyzed for 6 hours while being gently stirred. The concentration of the guanidine hydrochloride solution of the external solution was slowly reduced over time in a stepwise manner into a PBS buffer wherein the fusion with a gold binding VH domain and a scFv specific to Troponin was refolded appropriately.

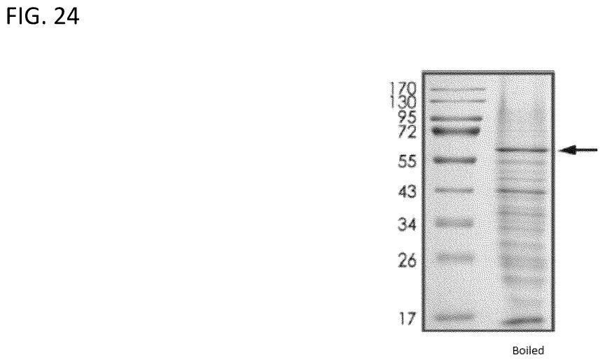

FIG. 24 shows Coomassie-stained SDS-PAGE gels indicating the expression and purity of bispecific antibody SEQ ID No: 29. The scFv Troponin fusion SEQ ID NO: 29 was then loaded onto a gold target surface as indicated using quartz crystal microbalance with dissipation monitoring (QCM-D).

The fusion protein and Troponin antigen was diluted in 1×PBS solution in Type 1 water to a concentration of 25 μg/ml. BSA was diluted to 100 μg/mL using the same PBS

### Solution

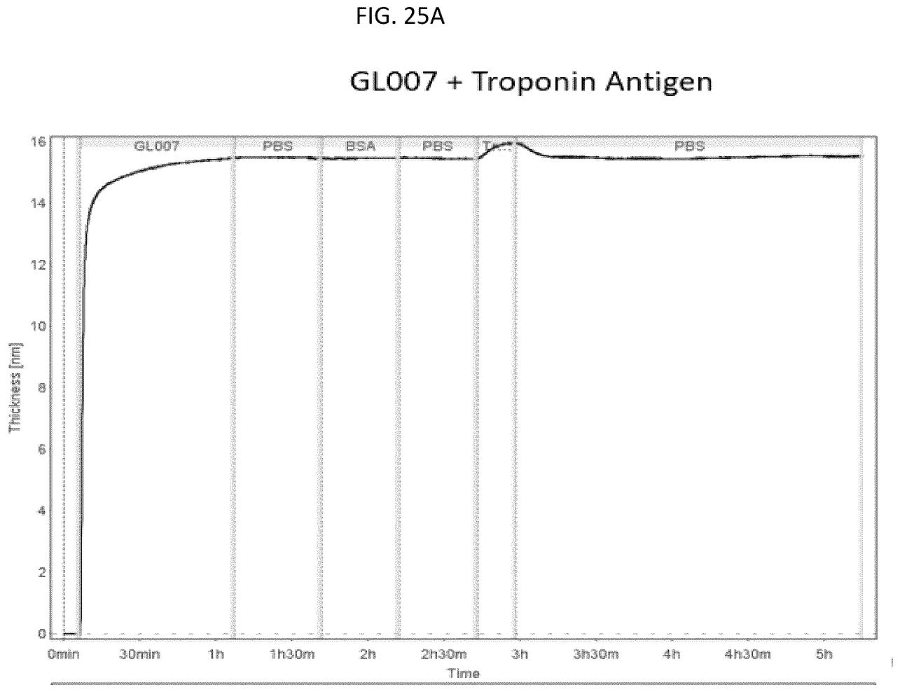

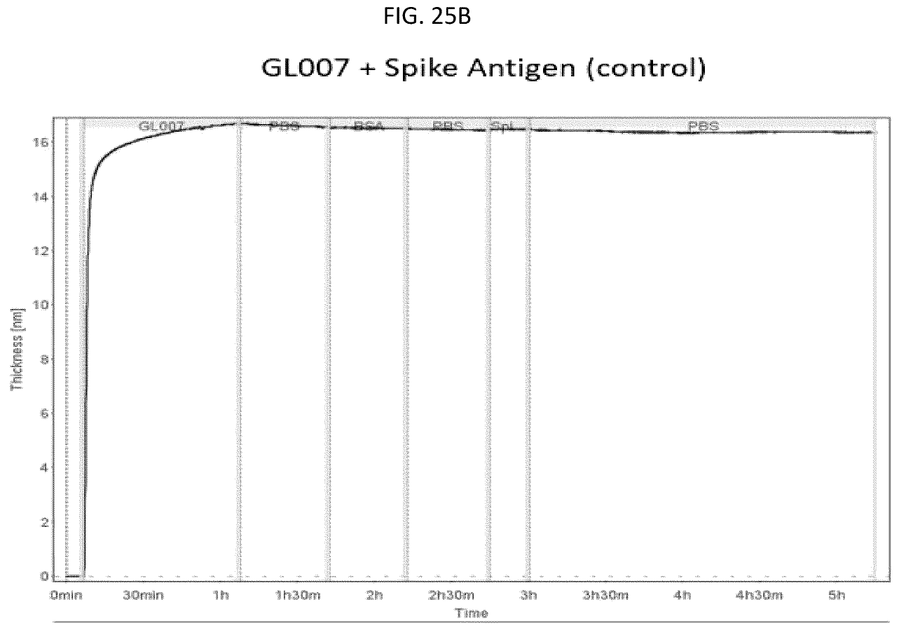

The QCM sensor was then primed with PBS for about 1 hr and then was washed with new PBS for 5 min. The scFv Troponin fusion diluted in PBS solution was loaded on two Gold surface sensors for 1 hr. After absorption of the fusion peptide to the surface, the sensors were washed with 30 min of PBS, followed by 30 min of BSA solution, followed by 30 min of PBS. Troponin antigen or Spike Antigen control was then added to the respective sensor for 15 min, followed by another 30 min wash of PBS. FIG. 25A-B shows the absorption changes under this protocol and Table 11 shows the change in mass and thickness values.

TABLE 11
GL007 scFv Troponin fusion Modeled Mass and Thickness Results

| | Sensor 1: Troponin Antigen | | | | Sensor 2: Spike Antigen | | | |
|---|---|---|---|---|---|---|---|---|
| | Sauer- | Sauer- | Visco- | Visco- | Sauer- | Sauer- | Visco- | Visco- |
| | Mass | Thickness | Mass | Thickness | Mass | Thickness | Mass | Thickness |
| Step | (ng/cm²) | (nm) | (ng/cm²) | (nm) | (ng/cm²) | (nm) | (ng/cm²) | (nm) |
| GL007 | 1857.3 | 15.5 | 2251.0 | 18.8 | 2003.8 | 16.7 | 2247.0 | 18.7 |
| PBS | 1856.6 | 15.5 | 2272.1 | 18.9 | 1987.7 | 16.6 | 2243.6 | 18.7 |
| BSA | 1857.5 | 15.5 | 2293.0 | 19.1 | 1982.2 | 16.5 | 2247.3 | 18.7 |
| PBS | 1854.0 | 15.5 | 2297.4 | 19.1 | 1974.5 | 16.5 | 2233.0 | 18.6 |
| Antigen | 1916.8 | 16.0 | 2382.6 | 19.9 | 1978.6 | 16.5 | 2249.1 | 18.7 |
| PBS | 1866.0 | 15.6 | 2318.2 | 19.3 | 1966.0 | 16.4 | 2225.1 | 18.5 |

After adsorption of GL007 on gold sensor, negligible thickness changes during subsequent PBS rinsing step and BSA blocking step are detected. While the Troponin Antigen shows some initial adsorption to sensor, minimal final troponin antigen adsorption was observed after PBS rinsing.

### EXAMPLE 19

Lateral Flow Assay Streptavidin Fusion Proteins:

GL011 was produced by initially being cloned and amplified in the recombinant baculovirus Sf9 insect cell system. The gene to GL011 was inserted into plasmid DNA as known in the art using the QIAGEN miniprep DNA purification kit. Sf9 cells were also seeded in insect cell medium in a six-well tissue culture plate and allowed to attach.

For transfection 0.2 micrograms of DNA, 0.8 micrograms of baculovirus transfer vector DNA, 4 microliters of Cellfectin reagent and 0.8 milliliters of FBS/antibiotics free medium was mixed and incubated at RT for 15 minutes. The medium from the cells was replaced with 2 milliliters of FBS/antibiotics free medium. The wash medium was removed and the transfection mix complex was overlaid onto the washed cells at 60 rpm, shaking for 4 hrs at 27° C. Once transfection of the recombinant baculovirus with GL011 gene, the baculovirus was amplified in T75 flasks with Sf9 cells per the SignalChem Pharmaceutical Sf9 amplification system.

To express the recombinants GL011 protein, 3×10⁸ Sf9 cells in 300 ml of Excell-400 medium from JHR Biosciences were combined with about 5 MOI baculovirus in a spinner flask for shaking at 80 RPM for 72 hrs at 27° C. The Sf9 cells are then harvested by centrifugation of the medium and the removal of the supernatant. The pellet is then lysed and purified with the His-Tag on the GL011 protein by using the Talon Cobalt beads system.

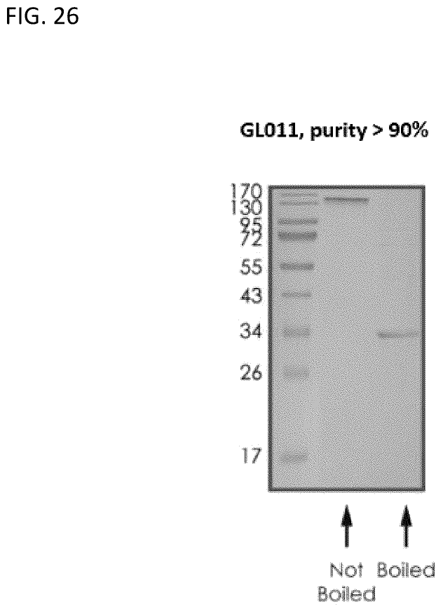

FIG. 26 shows the purity of the GL011 His-tagged gold-binding streptavidin fusion proteins on a Coomassie-stained SDS-PAGE gel. The amino acid sequence of GL011 is confirmed with the following Sequence: Affinity tag (his-tag)-Fusion of streptavidin to linker (SEQ ID NO:1) to Gold Protein (98 aa Gold protein).

TABLE 12
GL011 Fusion Sequence

| ID | Target | Fusion Sequence |
|---|---|---|
| GL011 | Gold | His-Tag- SEQ ID NO: 22- |
| | | SEQ ID NO: 1-SEQ ID NO: 4 |

Gold-binding streptavidin fusion protein GL011 was then conjugated to gold nanoparticles according to the method outlined in Example 5.

Immobilization of biotinylated detection antibodies onto gold-binding streptavidin fusion protein coated gold nanoparticles and their antigen binding capacity was then tested using a lateral flow assay. In this assay, the antigen, (SARS-CoV-2 Nucleocapsid antigen), was directly dotted on the strip membrane at various concentrations of antigen. Specifically, SARS-CoV-2 Nucleocapsid antigen was diluted in human pooled saliva at 100 ng/mL, 10 ng/mL, 2 ng/mL and then individually spotted on the lateral flow assay membrane.

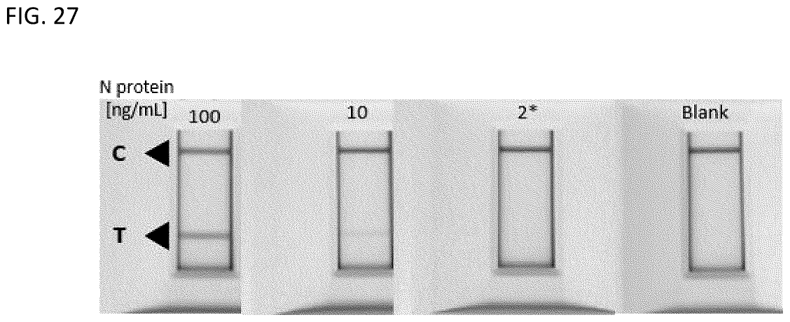

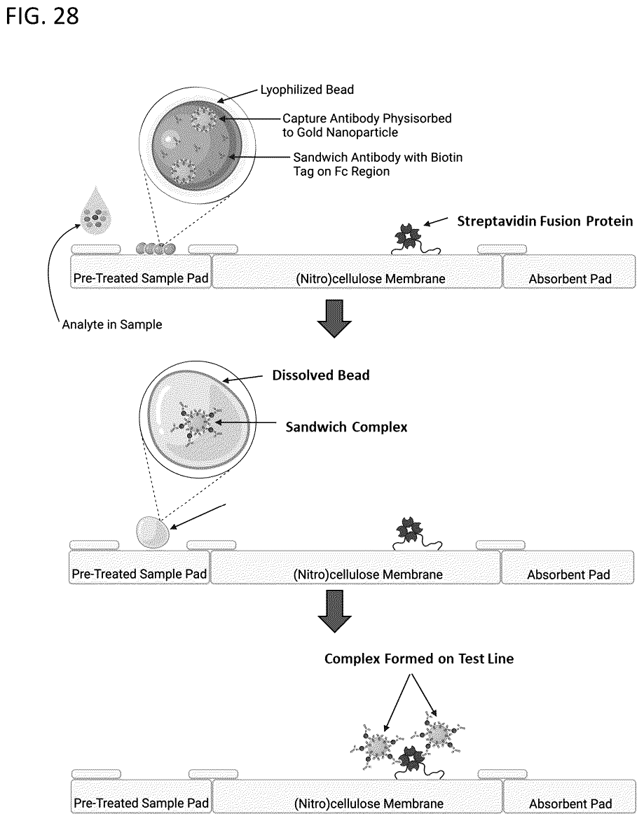

Biotinylated detection antibody (SARS-CoV-2 nucleocapsid antibodies) was loaded onto streptavidin fusion protein GL011 immobilized on gold nanoparticles, and then allowed to flow up the membrane. As shown in FIG. 27, with immobilized Nucleocapsid antigen, the strips can be detected by GL011 conjugate (i.e. gold nanoparticle-streptavidin fusion protein-biotin conjugated detection antibody complex) in a lateral flow assay, being detected at as low a concentration of 2 ng/mL and specifically as indicated with the blank control with no Nucleocapsid antigen. These results show GL011 conjugate coupled to nucleocapsid antibody in a striped lateral flow assay successfully detects the nucleocapsid antigen in artificial saliva.

In another example with GL011, the Gold-binding streptavidin fusion protein GL011 was again conjugated to gold nanoparticles according to the method outlined in Example 5. Biotinylated detection antibody (SARS-CoV-2 nucleocapsid antibodies) was then loaded onto the streptavidin fusion protein GL011 immobilized on gold nanoparticles.

In this assay, a rapid lateral flow assay, SARS-CoV-2 nucleocapsid antigen from a human anterior nasal swab sample was detected. Specifically, a control line polyclonal anti-IgG antibody was lined on the surface of the test strip. In addition, a test line comprising an anti-covid antibody was lined on the surface of the test strip.

For test preparation, the nasal swab sample was added to a buffer in the test tube for sample collection. The buffer includes the biotinylated detection antibody (SARS-CoV-2 nucleocapsid antibodies) loaded onto the conjugated GL011 fusion protein. After incubation, the sample was then added to the sample well of the test trip, allowing lateral flow of the sample on the test strip. After full lateral flow, the test detected SARS-CoV-2 nucleocapsid antigen in human samples with at least 84% sensitivity, and 99% specificity.

### EXAMPLE 20

Lateral Flow Assay:

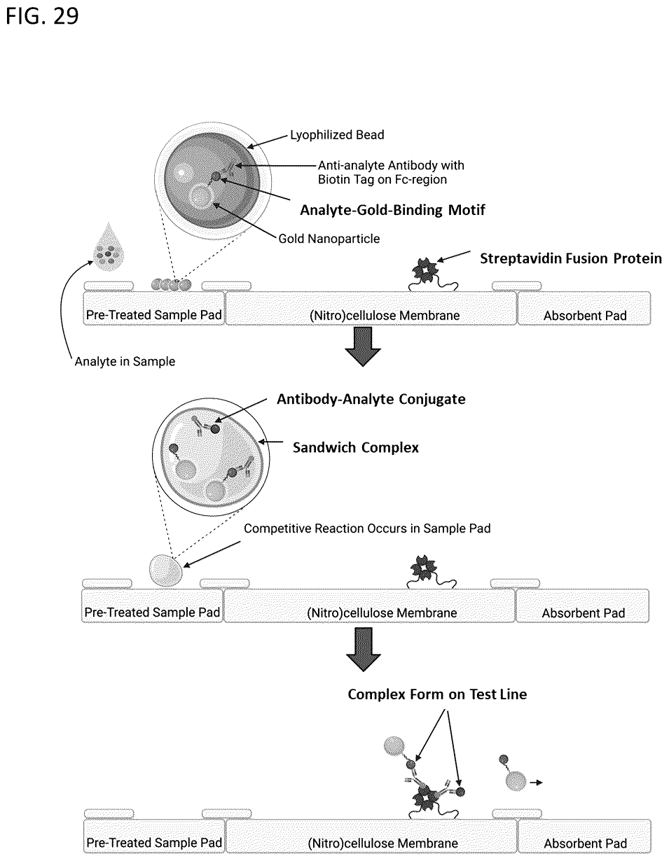

An exemplary assay that utilizes (nitro)cellulose-binding streptavidin fusion proteins (e.g., EMT032-EMT033) and dual-affinity probes for detecting or quantifying the analyte level (SARS-CoV-2 Nucleocapsid protein) in a test sample is described. The assay is manufactured into a diagnostic kit using the dual-affinity probes according to the methods described above. As shown in FIG. 29, the kit has a pre-treated sample pad section for collecting and reacting the biological sample from a subject; nitrocellulose membrane portion where the detection complex is formed on the test line; and an absorbent pad at the end of the strip to collect the flow-through. In this particular example, the pre-treated sample pad comprises an analyte composition comprising lyophilized beads that comprise two components: 1) encapsulated physisorbed antibody-gold nanoparticles wherein the antibody is a capture element antibody that specifically binds to the analyte SARS-CoV-2 Nucleocapsid protein; and 2) a free biotinylated antibody that also specifically binds to a different epitope of the analyte, SARS-CoV-2 Nucleocapsid protein. The sample pad is treated with lyophilized beads immediately prior to using the kit, or alternatively, the beads are added during the manufacturing of the kit.

The test sample having the analyte of interest is collected from a subject by using a variety of collection methods (e.g., collecting spat sample in a tube, taking a nasal swap using the assay strip, etc.) and the collected test sample is introduced to a reagent solution. The reagent solution comprising the test sample is then directly applied onto the pre-treated sample pad.

The sample including the analyte lateral flows to and then contacts the lyophilized beads, allowing the beads will dissolve in the sample reagent and form a sandwich of biotinylated analyte-bound antibody bound to the analyte and the analyte also bound to the antibody conjugated gold nanoparticles, having the analyte SARS-CoV-2 Nucleocapsid protein sandwiched in between both antibodies. These sandwich complexes form in the sample pad before the sample flow begins, resulting in more controllable stoichiometry and reduced margins of error. The sandwich complex then flows laterally on the surface of the nitrocellulose membrane where they are captured on the test line by forming complexes (i.e., detection conjugates) with immobilized streptavidin fusion proteins. The presence of the detection conjugate captured at the test line is observed with the naked eye to determine the presence and/or quantity of the analyte in the test sample.

### EXAMPLE 21

Competitive Sandwich Lateral Flow Assay:

Referring to FIG. 29, this assay is performed to evaluate the presence or quantity of the analyte of interest (SARS-CoV-2 Nucleocapsid protein) in a test sample. First, similar to the sample collection protocol described in Example 20, the test sample is collected from a subject, mixed with the reagents, applied onto the pre-treated sample pad that comprises lyophilized beads comprising a dual-affinity probe. The dual-affinity probe includes biotinylated gold nanoparticles that are coated with the antigen (recombinant SARS-CoV-2 Nucleocapsid protein that is not from the sample) that is engineered to be immobilized on the gold through its gold-binding motif. The antigen also is bound to a capture element biotinylated antibody that is designed to specifically bind competitively to the antigen and the SARS-CoV-2 Nucleocapsid protein from the sample. Once the sample is applied to the pre-treated sample and laterally follows to the lyophilized beads, the lyophilized beads dissolve in the sample reagent, and this competitive binding reaction occurs (i.e., the analyte in the sample and the gold-immobilized antigen from the beads competitively bind to anti-analyte antibody). The lyophilized beads lateral flow down the surface, resulting in more controlled stoichiometry and reduced margins of error. The gold nanoparticle coating antigen bound biotinylated antibody complexes, along with the analyte bound biotinylated antibody, will then flow laterally on the surface of the nitrocellulose membrane where they are captured on the test line by forming complexes with immobilized streptavidin fusion proteins. The more analyte in the sample will displace the more antibody from the gold nanoparticles, thus the less gold captured complexes are observed on the test line when analyte SARS-CoV-2 Nucleocapsid protein is present in the sample. The quantity of the analyte in the sample is observed with the naked eye by determining the intensity of the signal.

### EXAMPLE 22

Multi-Bead Sandwich Lateral Flow Assay:

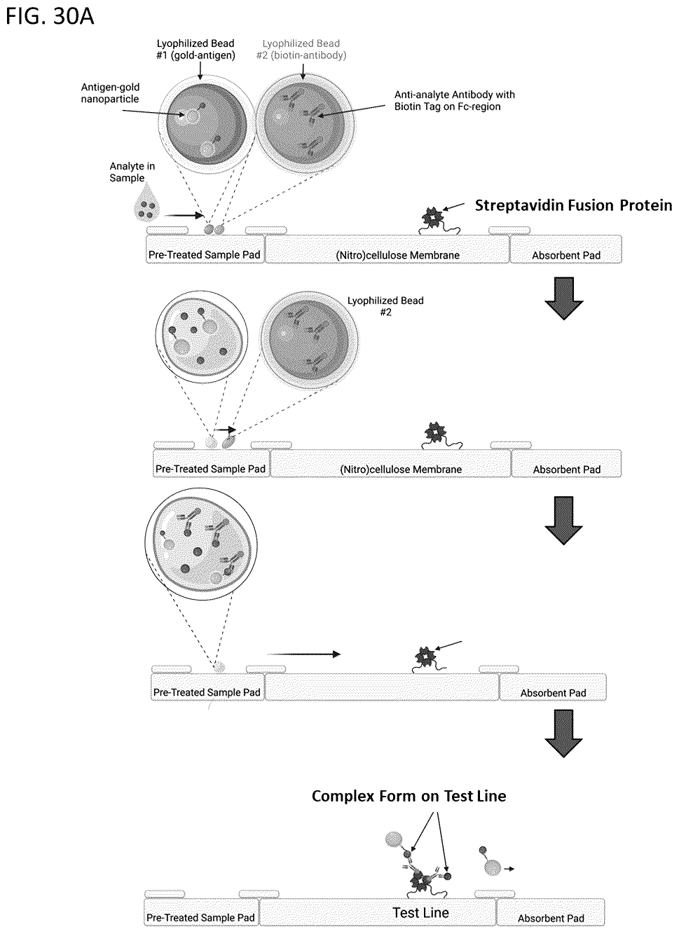

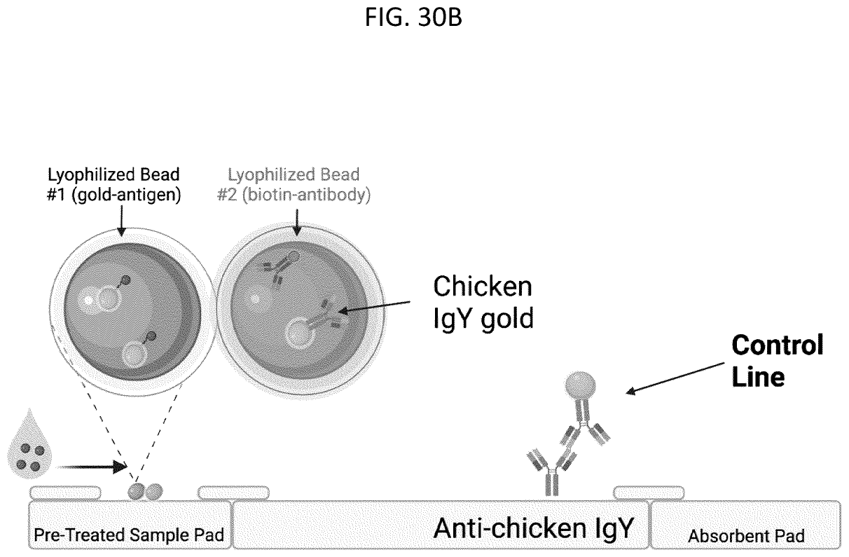

This assay kit uses two different analyte compositions of lyophilized beads for pre-treating the sample pad of the strip of the assay. An exemplary embodiment is shown in FIGS. 30A-B. As shown in FIG. 30A, the sample comprising the analyte of interest (SARS-CoV-2 Spike protein) is collected with a reagent and applied onto the pre-treated sample pad. The sample laterally flows down the sample pad wherein the sample reagent dissolves the first analyte composition of lyophilized beads. The first analyte composition of lyophilized beads comprises gold nanoparticles coated with immobilized antigen (recombinant SARS-CoV-2 Spike protein that is not from the sample) fused to a gold binding protein. As the analytes from the sample and the antigen-bound gold nanoparticles mix in the sample reagent, the mixture additionally comes into contact with the second analyte composition of lyophilized beads comprising biotinylated capture element antibody that binds specifically to the antigen and the analyte. Once in contact, the beads of the second analyte composition dissolve and release the biotinylated antibody, wherein the analyte gold beads and analyte competitively bind to the biotinylated capture element antibody. The excess of analyte in the mixture outcompetes the gold immobilized antigen for antibody binding.

This complex formation occurs in the sample pad before the lateral flow begins, resulting in more controlled stoichiometry and reduced margins of error. The gold nanoparticle-antigen bound biotinylated antibody complexes, along with the analyte bound biotinylated antibody, then flows laterally on the surface of the nitrocellulose membrane where they are captured on the test line by forming complexes with immobilized streptavidin fusion proteins. A positive control can also run alongside with the sample by adding gold nanoparticle conjugated chicken IgY antibody into the second group of lyophilized beads and immobilizing anti-chicken IgY antibody onto the cellulose membrane on the control line.

The more analyte in the sample will capture the more antibody out competing from the antigen bound gold nanoparticles, thus less gold captured complexes are observed on the test line when analyte is provided in the sample. The quantity of the analyte in the sample is observed with the naked eye by determining the intensity of the signal.

### EXAMPLE 23

Multi-Bead Sandwich Lateral Flow Assay:

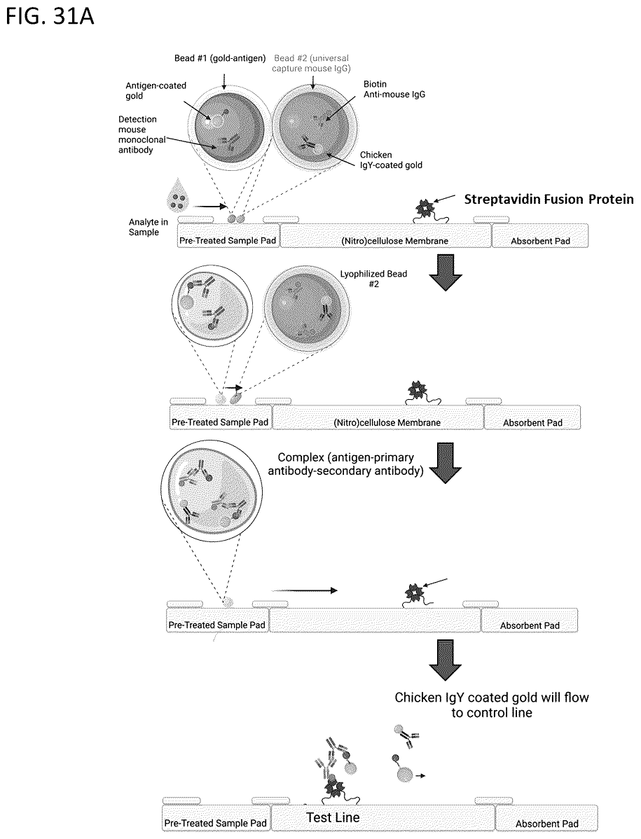

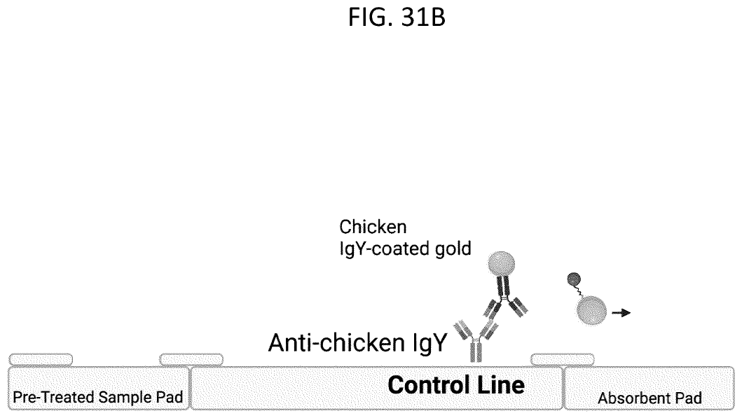

In another exemplary embodiment, the sample collection pad of the assay kit is pre-treated with two different analyte compositions, each comprising lyophilized beads. The first analyte composition of beads include antigen (recombinant SARS-CoV-2 Spike protein that is not from the sample) fused to a gold binding protein that is coated with gold nanoparticles. The analyte also is bound to a non-biotinylated IgG mouse antibody that is bound to the antigen, and is capable to also bind the analyte. The second analyte composition includes lyophilized beads having i) a universal capture biotinylated anti-mouse IgG antibody and ii) a control of chicken IgY-coated gold nanoparticles. As shown in FIGS. 31A-31B, the two analyte compositions are treated adjacently on the pre-treated sample pad. The sample comprising analyte (SARS-CoV-2 Spike protein) is collected and applied onto the pre-treated sample pad where the sample reagent will dissolve the first analyte composition of lyophilized beads. As the analyte from the sample compete with gold-immobilized antigen for antibody binding, the second group of beads dissolves to release the secondary antibody and form biotinylated antigen-primary antibody-secondary antibody complexes. The complexes, along with chicken IgY antibody and the excess of unbound antigen-coated gold nanoparticles, then flow laterally on the surface of the nitrocellulose membrane where they are captured in the test line by forming complexes with immobilized streptavidin fusion proteins. Alternatively, the chicken IgY released from the second group of beads are captured on the control line by immobilized anti-chicken IgY antibody (FIG. 31B).

### EXAMPLE 24

Multi-Bead Sandwich Lateral Flow Assay:

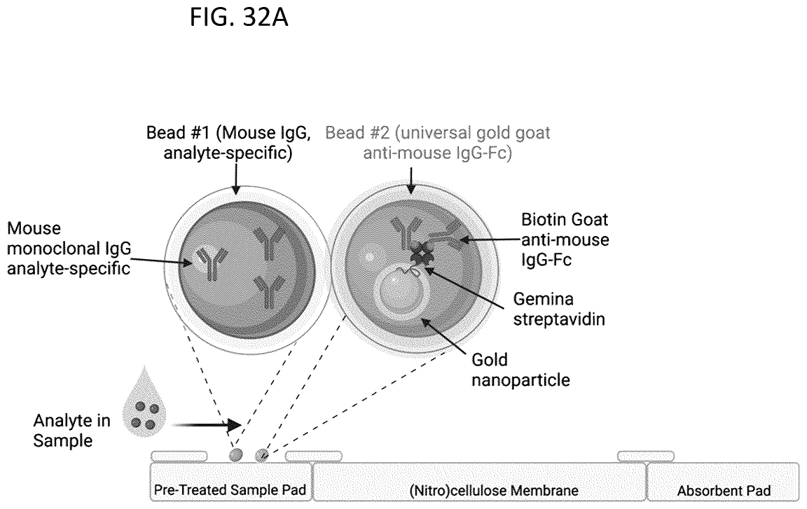

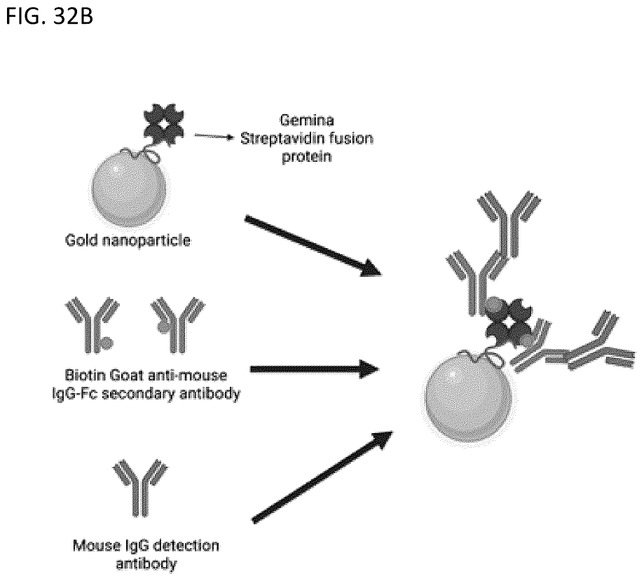

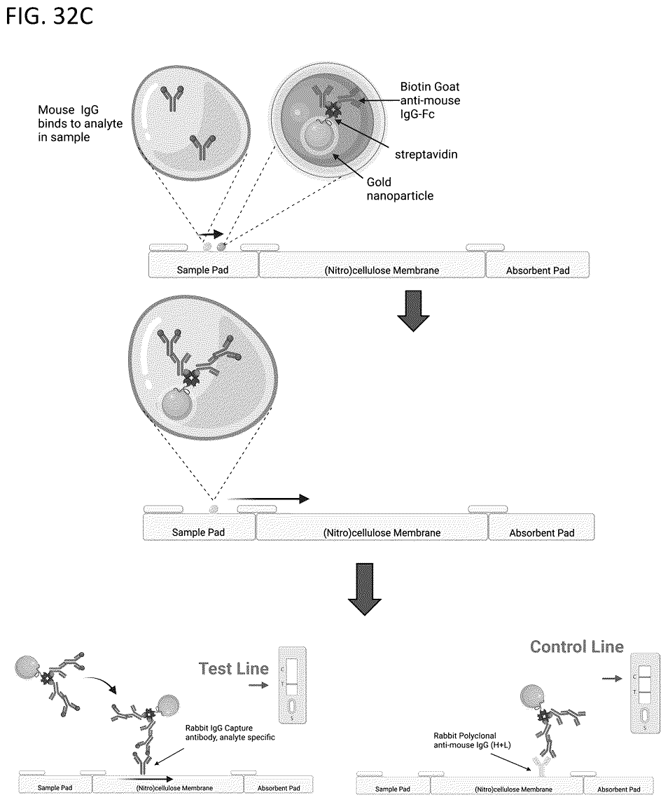

This is another exemplary assay kit for detecting the presence of analyte in a sample. In this kit, as shown in FIG. 32A, the sample collection pad is treated with two analyte compositions of lyophilized beads: i) having mouse monoclonal IgG antibody (i.e., anti-analyte SARS-CoV-2 Spike protein antibody); ii) having complexes of gold nanoparticles bound to streptavidin fusion proteins by gold binding protein conjugated biotinylated anti-mouse IgG antibody. As described in Example 1, the sample is collected from a subject by using a variety of collection methods, and the collected sample in the reagent is applied onto the sample pad. Upon dissolving of the first beads of the first analyte composition in the sample reagent, the analyte SARS-CoV-2 Spike protein in the sample is be recognized by the mouse monoclonal IgG antibody and then allowed to interact with the secondary mouse antibody that is in the second analyte composition comprising the complex with streptavidin fusion proteins and gold nanoparticles (FIGS. 32B-C). A positive assay control is detected on the control line when the excess of the complexes having the analyte-unbound primary mouse antibody forms complexes with the immobilized rabbit polyclonal anti-mouse IgG antibody. The presence of the detection conjugate captured at the test line is observed with the naked eye to determine the presence and/or quantity of the analyte in the test sample.

### EXAMPLE 25

Sandwich Lateral Flow Assay Application of Gold-Binder Protein G Complexes:

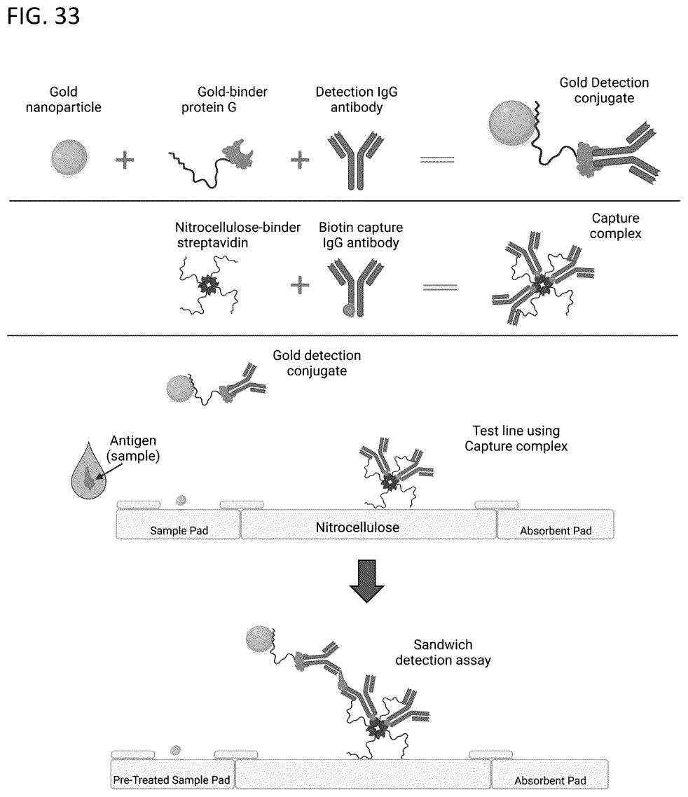

Referring to FIG. 33, another exemplary sandwich detection complex is described. In this assay, the sample collection pad comprises a dual-affinity probe with a gold detection conjugate that is formed by indirectly binding the analyte capture element detection IgG antibody onto the gold nanoparticles using a protein G-gold binding motif fusion protein. The protein G-gold binding motif fusion protein is fused to the gold particle thereby allowing the protein G to bind to the IgG antibody that is specific to the analyte (SARS-CoV-2 Spike protein). On the test line of the assay, a nitrocellulose-binder streptavidin fusion protein is bound to a biotinylated IgG capture antibody. This biotinylated IgG capture antibody is also specific to the analyte, but at a different epitope as the IgG antibody from the dual-affinity probe. As described in Example 1, the sample collected from a subject by using a variety of collection methods is applied onto the sample pad where the analyte (or antigen) in the sample reagent interacts with gold detection conjugates to form a complex. The analyte complex comprising the SARS-CoV-2 Spike protein then laterally flows through the nitrocellulose membrane and is captured by the streptavidin fusion protein conjugated antibody. The presence of the detection conjugate captured at the test line is observed with the naked eye to determine the presence and/or quantity of the analyte in the test sample.

### EXAMPLE 26

Sandwich Lateral Flow Assay:

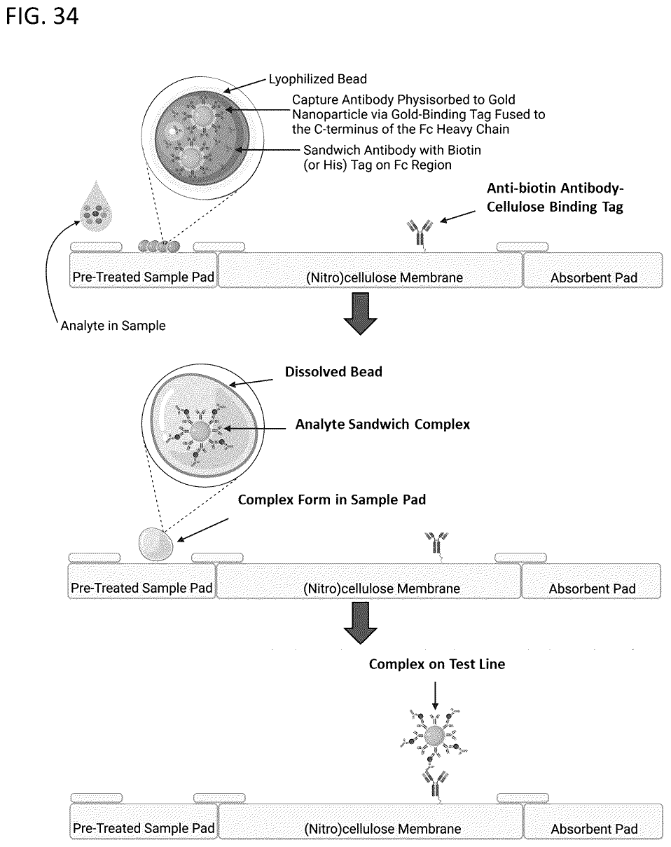

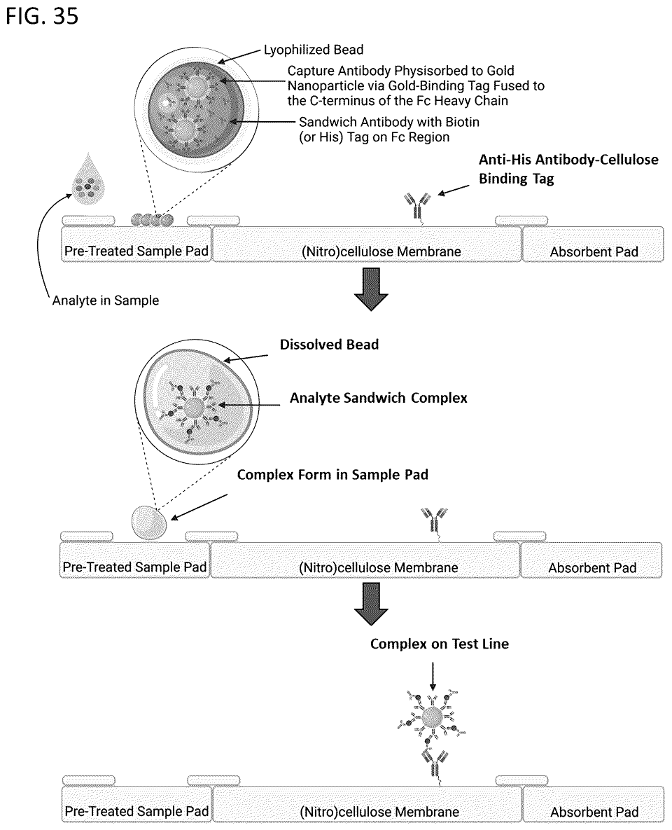

Another exemplary assay using biotinylated sandwich anti-analyte antibody and anti-biotin antibody immobilized onto the nitrocellulose membrane is described. As shown in FIGS. 34-35, the assay kit has a pre-treated sample pad section for collecting and reacting the sample from a subject; nitrocellulose membrane portion where the sandwich complex is formed on the test line; and an absorbent pad at the end of the strip to collect the flow-through in excess. In this particular example, the pre-treated sample pad comprises an analyte composition comprising a dual-affinity probe that includes two components: i) gold surface particles bound to physisorbed analyte capture antibody with a gold binding peptide; and ii) a tagged sandwich antibody that is also specific to binding the analyte SARS-CoV-2 Spike protein. Both antibodies are designed to bind different epitopes of the analyte SARS-CoV-2 Spike protein, thereby allowing for a full sandwich when the analyte is present.

The test sample having analyte of interest is collected from a subject by using a variety of collection methods (e.g., collecting spat sample in a tube, taking a nasal swap using the assay strip, etc.) and the collected test sample is directly applied onto the pre-treated sample pad or premixed with the kit reagent in a separate tube prior to applying. When the sample reagent contacts the lyophilized beads, the beads dissolve and allow the analyte SARS-CoV-2 Spike protein in the sample to form full antibody sandwich complexes. The sandwich complexes that are formed in the sample pad, then begin to laterally flow on the surface of the membrane. On the test line, the sandwich complexes are captured by binding the tagged antibody with an anti-Biotin (FIG. 34) or anti-His (FIG. 35) antibodies via the biotin or His tag on the tagged sandwich antibodies. The presence of the sandwich detection conjugate captured in the test line is observed with the naked eye to determine the presence and/or quantity of the analyte in the test sample.

### EXAMPLE 27

Antibody-Based Sandwich Lateral Flow Assay:

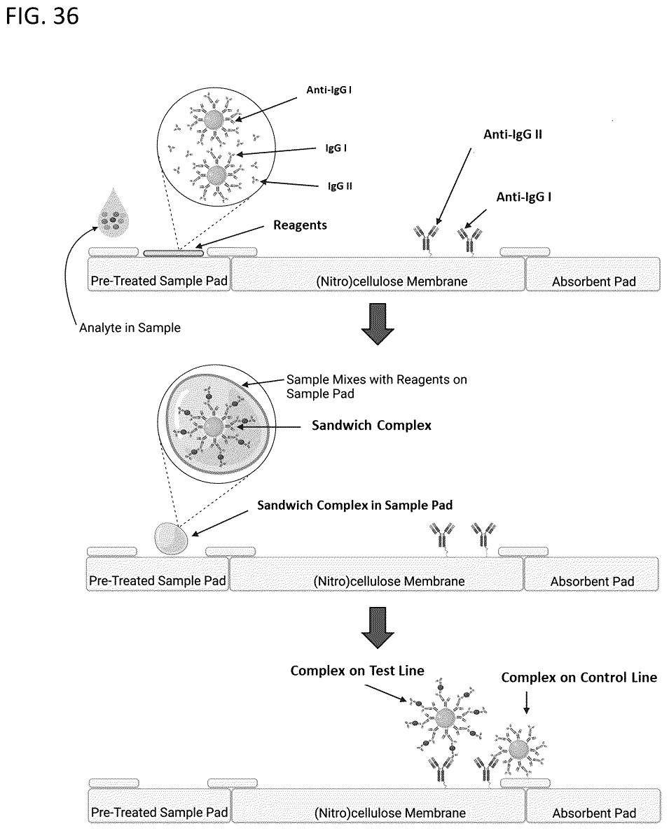

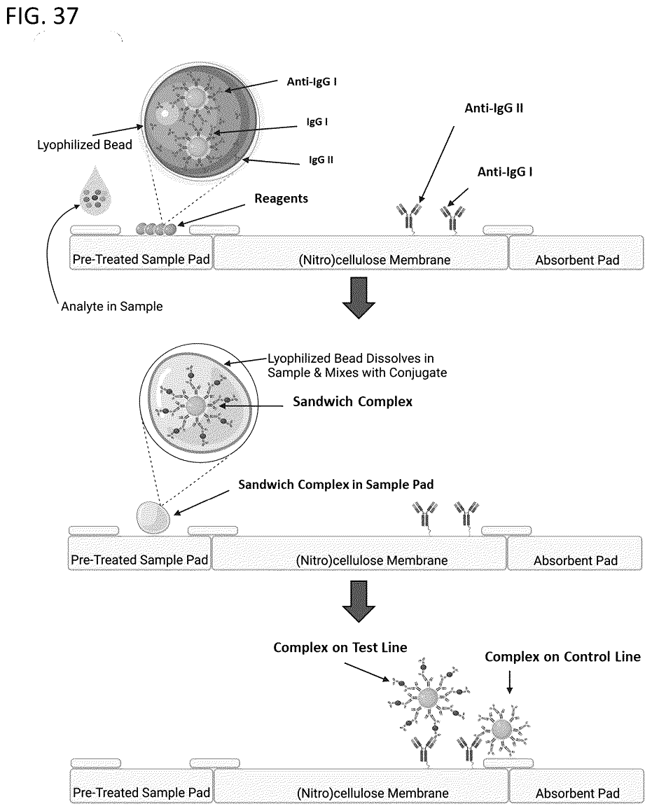

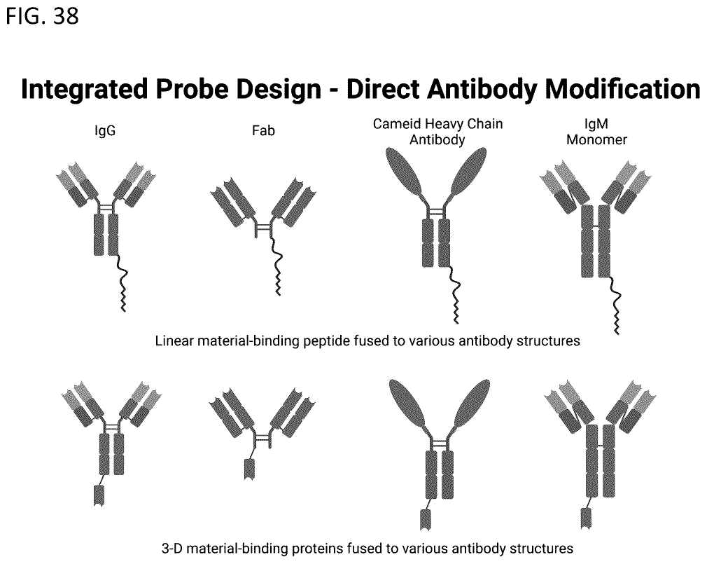

Another exemplary assay utilizes antibodies that were grown in two different species (such as two different species such as mouse and chicken or two different IgG antibody isotypes (e.g., IgG1 and IgG2)) for detecting analyte SARS-CoV-2 Spike protein in a sample. In a particular embodiment, a biological sample from a subject is placed onto the pre-treated sample pad where the pad holds an analyte composition containing a dual-affinity probe comprising a physisorbed secondary antibody having gold-binding peptides to bind to the gold nanoparticles. This secondary antibody is used to bind to a single species such as IgG1. The anti-IgG1 antibody is bound to an IgG1 antibody that is a primary anti-analyte antibody and can specifically bind to the analyte SARS-CoV-2 Spike protein. The analyte composition includes a second binding moiety which is a different species antibody than the primary anti-analyte antibody (in this case, is IgG2 antibody) that binds that can specifically bind to the analyte SARS-CoV-2 Spike protein at a different epitope as the IgG1 antibody. The sample comprising the analyte is added to the pre-treated sample and then laterally flows to the analyte composition on the pre-treated sample pad, there are two different species of anti-analyte antibodies to bind the analyte sandwich and form a full antibody sandwich. As shown in FIGS. 36-37, the analyte composition can include the dual-affinity probe with lyophilized beads or not lyophilized beads, and added multiple ways to the pre-treated sample pad, including using a different surface or same surface as the surface for the sample. Once the sample flows to the analyte composition, the sandwich complex is formed due to analyte being present and then laterally flows down the nitrocellulose membrane, where the complexes are captured by the immobilized secondary antibodies (anti-IgG1 and anti-IgG2 antibodies) on the nitrocellulose membrane. The analyte containing sandwich is captured and detected on the test line, whereas the unbound (no analyte) are detected on the control line (anti-IgG1). The presence of the sandwich detection conjugate captured at the test line may be observed with the naked eye to determine the presence and/or quantity of the analyte in the test sample.

### EXAMPLE 28

Lateral Flow Assay:

An exemplary assay that utilizes cellulose-binding streptavidin fusion proteins (e.g EMT033, GL018, GL019, and GL020) for detecting or quantifying the analyte level (SARS-CoV-2 Nucleocapsid protein) in a test sample is described.

EMT033 cellulose-binding streptavidin fusion protein was striped on nitrocellulose and cellulose strips for the immobilization of a biotinylated SARS-CoV-2 anti-nucleocapsid antigen capture antibody.

Two specific assays were performed: a wet assay and a dry assay.

In the wet assay, gold beads were conjugated with 2 distinct SARS-CoV-2 anti-nucleocapsid detection antibodies and mixed with biotinylated anti-nucleocapsid antibody, buffer and nucleocapsid antigen. Three different concentrations of nucleocapsid antigen were tested: 0 ng/mL, 37.5 ng/mL and 300 ng/mL.

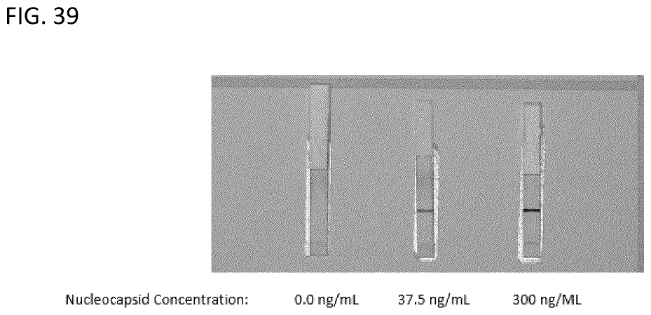

FIG. 39 shows the wet assay results when the EMT033 cellulose-binding streptavidin fusion protein was striped on nitrocellulose at all three concentrations of nucleocapsid antigen, indicating a much stronger signal at the 300 ng/mL concentration.

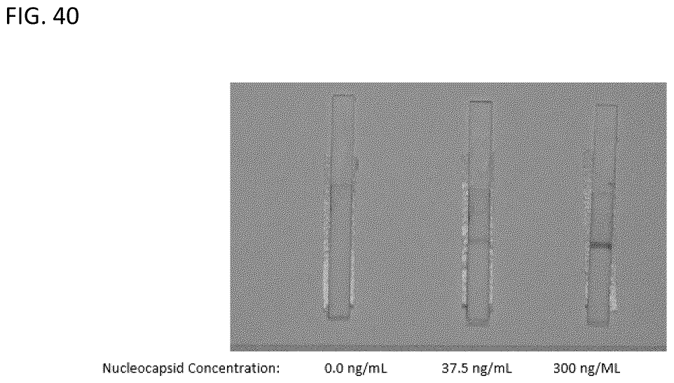

FIG. 40 shows the wet assay results when the EMT033 cellulose-binding streptavidin fusion protein was striped on cellulose (Whatman 43 paper) at all three concentrations of nucleocapsid antigen, indicating a much stronger signal at the 300 ng/mL concentration.

In the dry assay, gold beads were conjugated with 2 distinct SARS-CoV-2 anti-nucleocapsid detection antibodies and then applied to and dried on a conjugate pad. Biotinylated SARS-CoV-2 anti-nucleocapsid antibody was then applied to and dried on a separate but adjacent glass fiber pad.

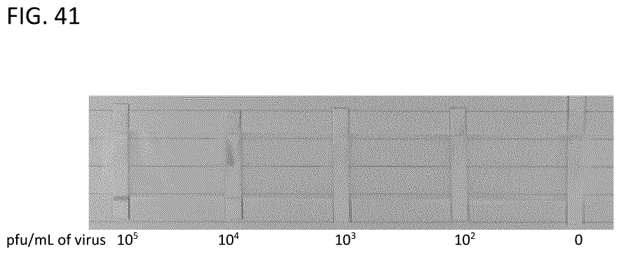

The dry assay was then run using both SARS-CoV-2 nucleocapsid antigen and various concentrations of UV-inactivated whole SARS-CoV-2 virus in running buffer. FIG. 41 shows the dry assay results when the EMT033 cellulose-binding streptavidin fusion protein was striped on cellulose (Whatman 43 paper), indicating that this cellulose-based dry lateral flow assay detects whole SARS-CoV-2 virus down to as low as 10³ pfu/mL of virus.

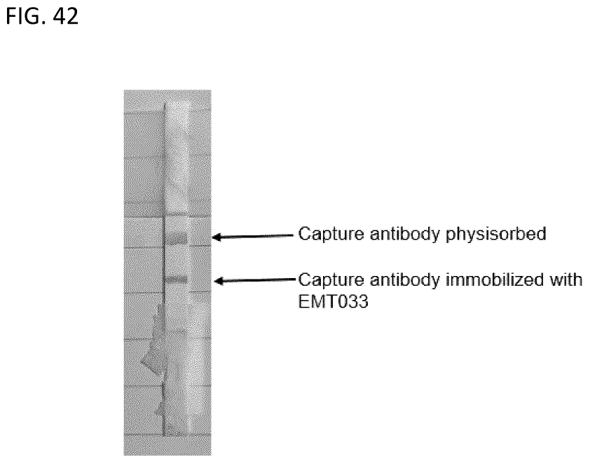

In a comparative dry assay, the same dry assay was applied as above but comparing EMT033 cellulose-binding streptavidin fusion protein striped on cellulose strips for the immobilization of a biotinylated SARS-CoV-2 anti-nucleocapsid antigen capture antibody, or the biotinylated SARS-CoV-2 anti-nucleocapsid antigen capture antibody was striped by physisorption on the cellulose strip, i.e., without any EMT033 cellulose-binding streptavidin fusion protein. FIG. 42 shows the results using a fixed concentration of SARS-CoV-2 nucleocapsid antigen, indicating detection of SARS-CoV-2 nucleocapsid antigen from both the use of a cellulose-binding streptavidin fusion protein or no fusion protein.

### EXAMPLE 29

PDMS-Binding Fusion Proteins to PDMS Surfaces Via QCM-D:

Dual affinity probes were created with affinity tag (his-tag)-Fusions of streptavidin to linker to various PDMS binding motifs, i.e., GL014 (SEQ ID NO: 56); GL015 (SEQ ID NO: 57); and GL016 (SEQ ID NO: 58) fusions. Assay conditions similar to Examples 1 and 6 above, but using a PDMS surface.

Specifically, all QCM-D analyses were performed at 23° C. on the 4-channel Qsense™ Analyzer instrument (Biolin Scientific, Gothenburg, Sweden). The PDMS Qsense™ sensor chips were rinsed and primed with PBS for 1 hour. An additional PBS was performed for 5 min. The GL014 (SEQ ID NO: 56); GL015 (SEQ ID NO: 57); and GL016 (SEQ ID NO: 58) fusions were then flowed over their respective PDMS sensor chips for 1 hr, followed by a 10 mM PBS wash step for 30 min, followed by a 30 min BSA block step and another PBS wash step for 30 min. Binding of the GL014; GL015; and GL016 fusions to the surface, was conferred using Qsense™ Dfind™ analysis software. To test as a dual affinity probe, biotinylated N-antigen antibody was then flowed over the respective fusions for 40 min, followed by a 10 mM PBS wash step for 30 min. SARS-CoV-2 nucleocapsid antigen was then flowed over each channel for 15 min, followed by a 10 mM PBS wash step for 30 min. The raw data was analyzed using Qsense™ Dfind™ analysis software using a Kelvin-Voigt viscoelastic model.

The thickness, the mass deposited, elasticity and viscosity of the resulting layers after each step were calculated and are summarized in Tables 13-15 for each fusion protein respectively.

TABLE 13
Thickness and viscoelastic properties with the GL014 protein fusion
on PDMS QSensor

| Experimental | Thickness | Mass | Shear Viscosity | Shear Elastic |
|---|---|---|---|---|
| Step | [nm] | [ng/cm²] | [μPa*s] | Modulus [kPa] |
| GL014 | 4.6 | — | — | — |
| PBS buffer- | 4.9 | — | — | — |
| Rinsing | | | | |
| BSA | 4.6 | — | — | — |
| PBS Buffer- | 4.7 | — | — | — |
| Rinsing | | | | |
| N-Antibody | 19.0 | 2280.6 | 2697.7 | 96.9 |
| PBS Buffer- | 20.2 | 2428.6 | 2420.8 | 53.0 |
| Rinsing | | | | |
| N-Antigen | 26.5 | 3167.2 | 3448.9 | 80.0 |
| PBS Buffer- | 26.2 | 3132.2 | 3292.9 | 50.4 |
| Rinsing | | | | |

TABLE 14
Thickness and viscoelastic properties with the
GL015 protein fusion on PDMS QSensor

| Experimental | Thickness | Mass | Shear Viscosity | Shear Elastic |
|---|---|---|---|---|
| Step | [nm] | [ng/cm²] | [μPa*s] | Modulus [kPa] |
| GL015 | 2.4 | — | — | — |
| PBS buffer- | 2.6 | — | — | — |
| Rinsing | | | | |
| BSA | 2.4 | — | — | — |
| PBS Buffer- | 2.6 | — | — | — |
| Rinsing | | | | |
| N-Antibody | 7.8 | 943.3 | 2598.6 | 55.8 |
| PBS Buffer- | 17.1 | 2054.7 | 1317.1 | 114.4 |
| Rinsing | | | | |
| N-Antigen | 23.9 | 2877.3 | 1631.3 | 23.7 |
| PBS Buffer- | 22.2 | 2658.4 | 1583.3 | 40.7 |
| Rinsing | | | | |

TABLE 15
Thickness and viscoelastic properties with the
GL016 protein fusion on PDMS QSensor

| Experimental | Thickness | Mass | Shear Viscosity | Shear Elastic |
|---|---|---|---|---|
| Step | [nm] | [ng/cm²] | [μPa*s] | Modulus [kPa] |
| GL016 | 2.2 | — | — | — |
| PBS buffer- | 2.2 | — | — | — |
| Rinsing | | | | |
| BSA | 2.7 | — | — | — |
| PBS Buffer- | 2.8 | — | — | — |
| Rinsing | | | | |
| N-Antibody | 12.5 | 1502.3 | 1793.1 | 77.7 |
| PBS Buffer- | 13.0 | 1569.9 | 1666.9 | 72.4 |
| Rinsing | | | | |
| N-Antigen | 18.9 | 2250.5 | 1781.0 | 93.4 |
| PBS Buffer- | 16.9 | 2024.2 | 1932.5 | 93.7 |
| Rinsing | | | | |

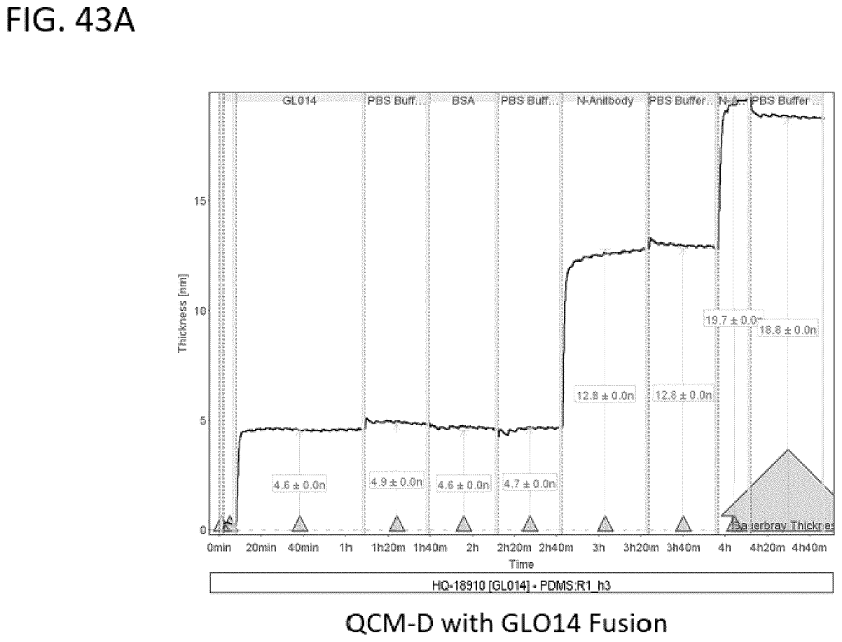

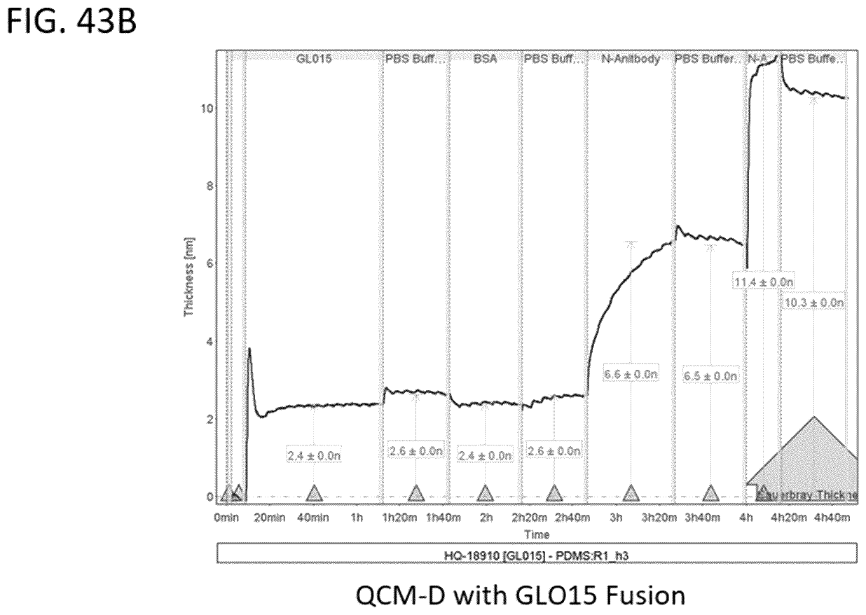

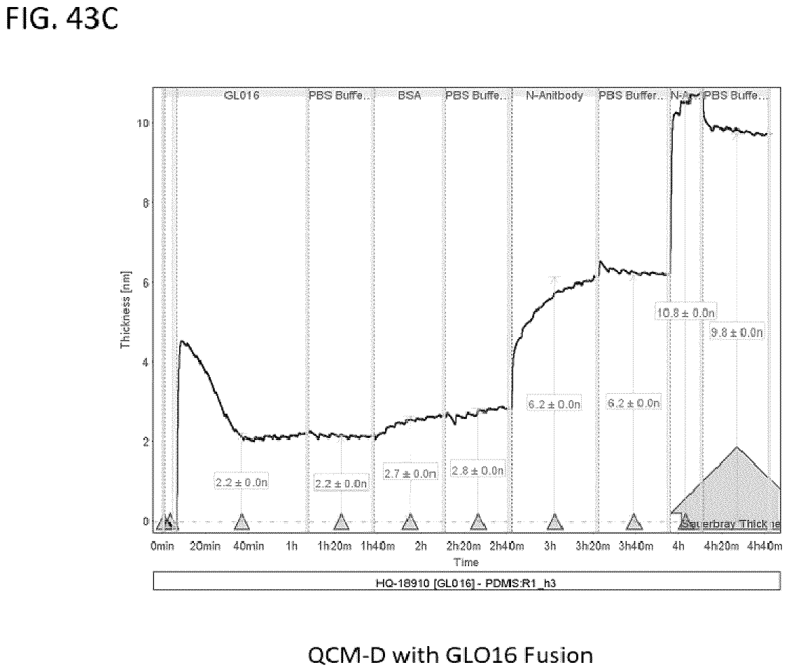

FIGS. 43A-C show the absorption changes under this protocol for each fusion portion GL0 14, GL0 15, and GL0 16 respectively. All of the PDMS-binding fusion proteins were found to bind to the PDMS sensor surface. A very small fraction of GL014 & GL016 washed off during the subsequent wash step and a larger fraction of GL015 washed off during the subsequent wash step. There were no significant changes to the thickness or mass of the layers during the subsequent blocking step with BSA or the washing steps. As can be seen in FIG. 43, a thick layer of biotinylated N-antibody was adsorbed onto the fusion protein, and a similar amount of N-antigen was adsorbed onto the N-antibody, indicating a sensitive and functional dual affinity probes using various PDMS fusion proteins on a QCM-D system.

While illustrative embodiments have been described above and illustrated in the accompanying drawings, it will be evident to those skilled in the art that modifications may be made without departing from this disclosure. Such modifications are considered as possible variants comprised in the scope of the disclosure.

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

1. A dual-affinity probe for detecting an analyte in a sample, the probe comprising a surface binding moiety (SBM), wherein the SBM is optionally an inorganic surface binding peptide (ISBP), and a capture element (CE), optionally wherein the probe comprises one or more polypeptides, and wherein the ISBP and the CE are present on the same or different polypeptides, optionally wherein the CE comprises a molecularly imprinted polymer (MIP), an aptamer, or an engineered protein.

2. The dual-affinity probe of claim 1, wherein the capture element (CE) is directly or indirectly connected to the SBM or ISBP, optionally via one or more linker (LI), wherein each LI may independent be a single bond or an amino acid sequence.

3. The dual-affinity probe of claim 1, wherein the immunoprobe has the following formula (Ia) or formula (IIa):
SBM-LI-CE (Ia) or
CE-LI-SBM (IIa).

4. The dual-affinity probe of claim 1, wherein the capture element CE is an organic binding entity specific for the analyte, wherein the analyte is optionally a pathogen or a fragment thereof.

5. The dual-affinity immunoprobe of claim 1, wherein the capture element comprises:
i. an antibody or an antigen-binding fragment thereof, optionally a single chain variable fragment (scFv) or a Fab fragment; or
ii. an antigen.

6. The dual affinity probe of claim 1, wherein LI is a single bond, or is selected from one or more of the group consisting of: a peptide or amino acid linker, an amino acid sequence comprising protein G from Streptococcus, and an amino acid sequence comprising streptavidin from Streptomyces.

7. The dual-affinity probe of claim 1, wherein the SBM or ISBP binds specifically to a biosensor material selected from the group consisting of polydimethylsiloxane (PDMS), zinc oxide (ZnO), gold, silica, silver, cellulose, plastic, polystyrene and graphene.

8. The dual affinity probe of claim 1, wherein the SBM or ISBP is selected from the group consisting of a binding peptide, a protein, an antibody with an affinity to the inorganic surface, and an immunogenic fragment thereof, optionally a single chain variable fragment (scFv) or a Fab fragment.

9. The dual affinity probe of claim 8, wherein the SBM or ISBP is a binding peptide, optionally selected from the group consisting of any peptide sequence of Table 1 herein.

10. (canceled)

11. The dual-affinity probe of claim 1, wherein the dual-affinity probe comprises a polypeptide having formula (IIIa) or (IIIb) and a polypeptide having formula (IVa) or (IVb):
SBM-LI-AL (IIIa);
AL-LI-SBM (IIIb);
ALB-LI-CE (IVa); or
CE-LI-ALB (IVb),
wherein AL is an active linker and ALB is an active linker binder.

12. The dual-affinity probe of claim 11, wherein AL is an amino acid sequence comprising protein G from Streptococcus or an amino acid sequence comprising streptavidin from Streptomyces.

13-16. (canceled)

17. The dual-affinity probe of claim 1, wherein the CE is an antigen, an antibody or an antigen-binding fragment thereof, optionally an scFv or an Fab, an aptamer, a MIP, or an engineered protein.

18. The dual-affinity probe of claim 17, wherein the CE is an antigen, antibody or an antigen-binding fragment thereof, wherein the antigen, antibody or antigen-binding fragment thereof is conjugated with biotin, and the LI includes an amino acid sequence comprising streptavidin from Streptomyces.

19. The dual-affinity probe of claim 18, wherein the CE is an antibody or an antigen-binding fragment thereof, and the LI is an amino acid sequence comprising protein G from Streptococcus.

20-23. (canceled)

24. The dual-affinity probe of claim 1, wherein LI is a single bond, or a peptide or amino acid linker.

25. The dual-affinity probe of claim 24, wherein the dual-affinity probe is a single fusion protein.

26-33. (canceled)

34. The dual-affinity probe of claim 2, wherein the SBM or ISBP is specific for gold, silica, silver, cellulose, plastic, polystyrene, or graphene.

35. The dual-affinity probe of claim 34, wherein the SBM or ISBP is specific for gold.

36-46. (canceled)

47. A method of determining the presence of and/or quantifying an analyte in a test sample, comprising:
i. contacting a test sample with the dual-affinity probe of claim 1, wherein the dual-affinity probe comprises a surface binding moiety (SBM) or an inorganic surface binding polypeptide (ISBP) and an analyte-specific capture element (CE), under conditions and for a time sufficient for analyte present in the test sample to bind to the analyte-specific capture element, thereby forming complexes comprising the analyte bound to the dual-affinity probe;
ii. determining the presence of and/or quantity of the complexes or analyte present in the complexes;
iii. wherein the presence of the complexes or the analyte in the complexes indicates the presence of the analyte in the test sample, and the quantity of the complexes or the analyte in the complexes indicates the quantity of analyte present in the test sample,
iv. thereby determining the presence of and/or quantifying the analyte in the test sample.

48-60. (canceled)

61. A dual affinity probe (DAP) for detecting an analyte in a sample, the DAP comprising i) a surface binding moiety (SBM) fused or bound to a non-analyte capture element (NACE), or ii) a surface particle fused or bound to a NACE or a capture element (CE).

62. The DAP of claim 61, wherein the NACE is one or more selected from the group consisting of a linker (LI), streptavidin, protein G, biotin, an antigen protein or peptide, or an antibody, or binding fragment thereof, wherein the antibody is a non-fragmented antibody, a modified antibody, a single chain variable fragment from an antibody, or a Fab fragment, and wherein the CE is an antibody or binding fragment thereof selected from the group consisting of: a non-fragmented antibody, a modified antibody, a camelid antibody, a single chain variable fragment, a Fab fragment, and an antigen protein or peptide.

63. The DAP of claim 61, wherein a surface particle is bound to a surface binding peptide (SBP), wherein the SBP is fused or bound to a non-analyte capture element (NACE), wherein the NACE is additionally fused or bound to a CE and the DAP has the following Formula I or Formula II:
(Surface particle)-SBP-NACE-CE (Formula Ic); or
(Surface particle)-SBP-CE (Formula IIc).

64. The DAP of claim 63, wherein the NACE comprises one or more selected from the group consisting of a linker, streptavidin, protein G, biotin, biotinylated protein, an antigen protein or peptide, and an antibody, or binding fragment thereof, optionally wherein the antibody is a non-fragmented antibody, a modified antibody, a camelid antibody, a single chain variable fragment, or a Fab fragment.

65. The DAP of claim 64, wherein the NACE comprises an antibody or binding fragment thereof, optionally a non-fragmented antibody, a modified antibody, a camelid antibody, a single chain variable fragment, or a Fab fragment, that specifically binds to IgG, e.g., an IgG1, IgG2, IgG3, an IgG4, IgM, IgD, and IgE, but preferably an IgG.

66-71. (canceled)

72. The DAP of claim 61, wherein the NACE comprises protein G and/or streptavidin.

73-90. (canceled)

91. A diagnostic kit for detecting an analyte in a sample, the kit comprising:
a) a surface,
b) a surface binding moiety (SBM) bound to at least a portion of the surface,
c) a reagent for collecting the sample;
and d) an analyte composition comprising a set of at least one dual affinity probe (DAP) of claim 1.

92-130. (canceled)

131. A dual affinity probe (DAP) for detecting an analyte in a sample, the DAP comprising:
i) a surface binding moiety (SBM), wherein the SBM is a gold binding protein (GBP), fused or bound to a non-analyte capture element (NACE), or
ii) a surface binding moiety (SBM), wherein the SBM is a gold binding protein (GBP), fused or bound to a capture element (CE).

132-160. (canceled)

161. A diagnostic kit for detecting an analyte in a sample, the kit comprising:
a) a surface;
b) a surface binding moiety (SBM) bound to at least a portion of the surface;
c) a reagent for collecting the sample; and
d) an analyte composition comprising a set of at least one dual affinity probe (DAP) of claim 1.

162-191. (canceled)

192. A diagnostic kit for detecting an analyte in a sample, the kit comprising:
a) a cellulose or nitrocellulose surface;
b) a surface binding moiety (SBM) comprising a cellulose or nitrocellulose binding moiety bound to at least a portion of the cellulose or nitrocellulose surface;
c) a reagent for collecting the sample; and
d) an analyte composition comprising: i) the dual affinity probe (DAP) of claim 1, wherein the DAP comprises a gold particle bound to an antibody wherein the antibody is specific for binding to an analyte and is bound to the gold particle by a gold binding protein, and ii) a second binding moiety antibody specific to the analyte and has binding tag.

193. The diagnostic kit of claim 192, wherein the DAP comprises SBM or ISBP that is a binding peptide, optionally selected from the group consisting of any peptide sequence of Table 1 herein. 194-

194-206. (canceled)

207. The method of determining the presence of and/or quantifying an analyte in a sample from a subject, by testing the sample with diagnostic kits of claim 91, comprising:
contacting a test sample with the reagent;
applying the test sample with the reagent to the surface thereby allowing the test sample to flow laterally on the surface thereby contacting the DAP composition on the surface;
allowing for the analyte present in the test sample to bind directly to the CE, thereby forming complexes comprising the analyte bound to the DAP;
allowing for the analyte complexed to the DAP to further flow laterally on the surface, thereby complexing to the surface binding moiety (SBM); and
wherein the presence of the DAP complexed to the SBM determines the presence and/or quantity of the analyte in the test sample.

208-226. (canceled)
